SEQUENCE LISTING (42 sequences)

SEQ ID NO: 1 - protein, 320 aa, Burkholderia cepacia
ADNYAATRYPIILVHGLTGTDKYAGVLEYWYGIQEDLQQRGATVYVANLSGFQSDDGPNGRGEQLLAYVKTVLAATGATKVNLVGHSQGGLTSRYVAAVAPDLVASVTTIGTPHRGSEFADFVQGVLAYDPTGLSSTVIAAFVNVFGILTSSSNNTNQDALAALKTLTTAQAATYNQNYPSAGLGAPGSCQTGAPTETVGGNTHLLYSWAGTAIQPTISVFGVTGATDTSTIPLVDPANALDPSTLALFGTGTVMVNRGSGQNDGVVSKCSALYGQVLSTSYKWNHLDEINQLLGVRGANAEDPVAVIRTHANRLKLAGV

SEQ ID NO: 2 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGATKVNLVGHSQGGLTSRYVAAVAPQLVASVTTIGTPHRGSEFADFVQGVLAYDPTGLSSTVIAAFVNVFGILTSSSHNTNQDALAALKTLTTAQAATYNQNYPSAGLGAPGSCQTGAPTETVGGNTHLLYSWAGTAIQPTISVLGVTGATDTSTIPLVDPANALDPSTLALFGTGTVMVNRGSGQNDGVVSKCSALYGQVLSTSYKWNHLDEINQLLGVRGANAEDPVAVIRTHANRLKLAGV

SEQ ID NO: 3 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGATKVNLVGHSQGGLTSRYVAAVAPQLVASVTTIGTPHRGSEFADFVQSVLAYDPTGLSSTVIAAFVNVFGILTSSSHNTNQDALAALKTLTTAQAATYNQNYPSAGLGAPGSCQTGAPTETVGGNTHLLYSWAGTAIQPTISVLGVTGATDTSTIPLVDPANALDPSTLALFGTGTVMVNRGSGQNDGLVSKCSALYGQVLSTSYKWNHLDEINQLLGVRGAYAEDPVAVIRTHANRLKLAGV

SEQ ID NO: 4 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGAQKVNLVGHSQGGLTSRYVAAVAPQLVASVTTIGTPHRGSEFADFVQSVLAYDPTGLSSAVIAAFVNVFGILTSSSHNTNQDALAALKTLTTAQAATYNQNYPSAGLGAPGSCQTGAPTETVGGNTHLLYSWAGTAIQPTISVLGVTGAKDTSTIPLVDPANALDPSTLALLGTGTVMVNRGSGQNDGLVSKCSALYGQVLSTSYKWNHLDEINQLLGVRGAYAEDPVAVIRTHANRLKLAGV

SEQ ID NO: 5 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGAQKVNLVGHSQGGLTSRYVAAVAPQLVASVTTIGTPHRGSEFADFVQSVLAYDPTGLSSAVIAAFVNVFGILTSSSHNTNQDALAALKTLTTAQAATYNQNYPSAGLGAPGSCQTGAPTETVGGNTHLLYSWAGTAIQPTISVLGVTGAKDTSTIPLVDPANALDPSTLALFGTGTVMVNRGSGQNDGLVSKCSALYGQVLSTAYKWNHLDEINQLLGVRGAYAEDPVAVIRTHANRLKLAGV

SEQ ID NO: 6 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGAQKVNLVGHSQGGLTSRYVAAVAPQLVASVTTIGTPHRGSEFADFVQSVLAYDPTGLSSTVIAAFVNVFGILTSSNHNTNQDALAALKTLTTAQAATYNQNYPSAGLGAPGSCQTGAPTETVGGNTHLLYSWAGTAIQPTISVLGVTGAKDTSTIPLVDPANALDPSTLALFGTGTVMVNRGSGQNDGLVSKCSALYGQVLSTAYKWNHLDEINQLLGVRGAYAEDPVAVIRTHANRLKLAGV

SEQ ID NO: 7 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGAQKVNLVGHSQGGLTSRYVAAVAPQLVASVTTIGTPHRGSEFADFVQSVLAYDPTGLSSTVIAAFVNVFGILTSSNHNTNQDALAALKTLTTAQAATYNQNYPSAGLGAPGSCQTGAPTETVGGNTHLLYSWAGTAIQPTISVLGVTGATDTSTIPLVDPANALDPSTLALLATGTVMVNRGSGQNDGLVSKCSALYGQVLSTSYKWNHLDEINQLLGVRGAYAEDPVAVIRTHANRLKLAGV

SEQ ID NO: 8 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGAQKVNLVGHSQGGLTSRYVAAVAPQLVASVTTIGTPHRGSEFADFVQSVLAYDPTGLSSTVIAAFVNVFGILTSSNHNTNQDALAALKTLTTAQAATYNQNYPSAGLGAPGSCQTGAPTETVGGNTHLLYSWAGTAIQPTISVLGVTGATDTSTIPLVDPANALDPSTLALLGTGTVMVNRGSGQNDGLVSKCSALYGQVLSTAYKWNHLDEINQLLGVRGAYAEDPVAVIRTHANRLKLAGV

SEQ ID NO: 9 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGAQKVNLVGHSQGGLTSRYVAAVAPQLVASVTTIGTPHRGSEFADFVQSVLAYDPTGLSSTVIAAFVNVFGILTSSSHNTNQDALAALKTLTTAQAATYNQNYPSAGLGAPGSCQTGAPTETVGGNTHLLYSWAGTAIQPTISVLGVTGAKDTSTIPLVDPANALDPSTLALLGTGTVMVNRGSGQNDGLVSKCSALYGQVLSTAYKWNHLDEINQLLGVRGAYAEDPVAVIRTHANRLKLAGV

SEQ ID NO: 10 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGATKVNLVGHSQGGLTSRYVAAVAPQLVASVTTIGTPHRGSEFADFVQSVLAYDPTGLSSAVIAAFVNVFGILTSSNHNTNQDALAALKTLTTAQAATYNQNYPSAGLGAPGSCQTGAPTETVGGNTHLLYSWAGTAIQPTISVLGVTGAKDTSTIPLVDPANALDPSTLALLGTGTVMVNRGSGQNDGLVSKCSALYGQVLSTSYKWNHLDEINQLLGVRGAYAEDPVAVIRTHANRLKLAGV

SEQ ID NO: 11 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGATKVNLVGHSQGGLTSRYVAAVAPQLVASVTTIGTPHRGSEFADFVQSVLAYDPTGLSSAVIAAFVNVFGILTSSNHNTNQDALAALKTLTTAQAATYNQNYPSAGLGAPGSCQTGAPTETVGGNTHLLYSWAGTAIQPTISVLGVTGAKDTSTIPLVDPANALDPSTLALFATGTVMVNRGSGQNDGLVSKCSALYGQVLSTSYKWNHLDEINQLLGVRGAYAEDPVAVIRTHANRLKLAGV

SEQ ID NO: 12 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGATKVNLVGHSQGGLTSRYVAAVAPQLVASVTTIGTPHRGSEFADFVQSVLAYDPTGLSSAVIAAFVNVFGILTSSSHNTNQDALAALKTLTTAQAATYNQNYPSAGLGAPGSCQTGAPTETVGGNTHLLYSWAGTAIQPTISVLGVTGAKDTSTIPLVDPANALDPSTLALFATGTVMVNRGSGQNDGLVSKCSALYGQVLSTAYKWNHLDEINQLLGVRGAYAEDPVAVIRTHANRLKLAGV

SEQ ID NO: 13 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGATKVNLVGHSQGGLTSRYVAAVAPQLVASVTTIGTPHRGSEFADFVQSVLAYDPTGLSSTVIAAFVNVFGILTSSNHNTNQDALAALKTLTTAQAATYNQNYPSAGLGAPGSCQTGAPTETVGGNTHLLYSWAGTAIQPTISVLGVTGAKDTSTIPLVDPANALDPSTLALLATGTVMVNRGSGQNDGLVSKCSALYGQVLSTSYKWNHLDEINQLLGVRGAYAEDPVAVIRTHANRLKLAGV

SEQ ID NO: 14 - protein, 320 aa, synthetic construct
ADNYAATRYPIILVHGLTGTDKYAGVLEYWYGIQEDLQQRGATVYVANLSGFQSDDGPNGRGEQLLAYVKTVLAATGATKVNLVGHSQGGLTSRYVAAVAPQLVASVTTIGTPHRGSEFADFVQSVLAYDPTGLSSTVIAAFVNVFGILTSSNHNTNQDALAALKTLTTAQAATYNQNYPSAGLGAPGSCQTGAPTETVGGNTHLLYSWAGTAIQPTISVLGVTGAKDTSTIPLVDPANALDPSTLALLGTGTVMVNRGSGQNDGLVSKCSALYGQVLSTAYKWNHLDEINQLLGVRGAYAEDPVAVIRTHANRLKLAGV

SEQ ID NO: 15-28 - skipped (14 entries)

SEQ ID NO: 29 - protein, 285 aa, Pseudomonas aeruginosa
STYTQTKYPIVLAHGMLGFDNILGVDYWFGIPSALRRDGAQVYVTEVSQLDTSEVRGEQLLQQVEEIVALSGQPKVNLIGHSHGGPTIRYVAAVRPDLIASATSVGAPHKGSDTADFLRQIPPGSAGEAVLSGLVNSLGALISFLSSGSTGTQNSLGSLESLNSEGAARFNAKYPQGIPTSACGEGAYKVNGVSYYSWSGSSPLTNFLDPSDAFLGASSLTFKNGTANDGLVGTCSSHLGMVIRDNYRMNHLDEVNQVFGLTSLFETSPVSVYRQHANRLKNASL

SEQ ID NO: 30 - protein, 296 aa, Pseudomonas fluorescens
MSQSTATRYPLVLVPGMLGFIRLLLYPYWYGIIKALRRGGATVIAVQVSPLNSTEVRGEQLLARIDEILRETGAARVNLFGHSQGSLTARYAAAKRPDLVASVTSVAGPNHGSELADYLQQHYPANTAKGRLLEALLRLIGWLMARLETGYHGPKLPVDIHASHQSLTREGVALFNQRYPQGLPETWGGQGPEVVNGVRYYSWSGTLQPGKTDRGGNLFDGTNRSCRLFAKTFVREPGQCDGMVGRYSSHLGTVIGDDYPLDHFDIVNQSLGLVGKGADPVRLFVEHAARLKAAGL

SEQ ID NO: 31 - protein, 319 aa, Burkholderia glumae
ADTYAATRYPVILVHGLAGTDKFANVVDYWYGIQSDLQSHGAKVYVANLSGFQSDDGPNGRGEQLLAYVKQVLAATGATKVNLIGHSQGGLTSRYVAAVAPQLVASVTTIGTPHRGSEFADFVQDVLKTDPTGLSSTVIAAFVNVFGTLVSSSHNTDQDALAALRTLTTAQTATYNRNFPSAGLGAPGSCQTGAATETVGGSQHLLYSWGGTAIQPTSTVLGVTGATDTSTGTLDVANVTDPSTLALLATGAVMINRASGQNDGLVSRCSSLFGQVISTSYHWNHLDEINQLLGVRGANAEDPVAVIRTHVNRLKLQGV

SEQ ID NO: 32 - protein, 319 aa, Chromobacterium viscosum; identical to SEQ ID NO: 31

SEQ ID NO: 33 - protein, 357 aa, Pseudomonas luteola
MRSRVVAGVACAMSVAPFADALMMAATTHVAMAATAPADNYATTRYPIILVHGLTGTDKYAGVLEYWYGIQEDLQQHGATVYVANLSGFQSDDGPKGRGEQLLAYVKTVLAATGATKVNLVGHSQGGLTSRYVAAVAPQLVASVTTIGTADRGSEFADFVQGVLAYDPTGLSSSVIAAFVNVFGILTSSSHNANQDALAGLKTLTTAQAATYNQNYPSAGLGRPGSCQTGAPTETVGGNTHLLYSWAGTAIQPTISVFGVTGATDTSTIPLIDPANVLDPSTLALFGTGTVMINRASGQNDGLVSKCSALYGKVLSTSYKWNHLDEINQLLGVRGANAEDPVAVIRTHANRLKLAGV

SEQ ID NO: 34 - protein, 296 aa, Pseudomonas fluorescens
MSQDSATRYPLVLVPGMLGFIRLVLYPYWYGIIKALRRGGATVIAVQVSPLNSTEVRGEQLLTRIDEILRETGAAKVNLFGHSQGSLTARYAAAKRPDLVASVTSVAGPNHGSELADYLAKYYPADSAKGRILEALLRSVGWLMALLETGYHGPKLPVDIHASHHSLTTEGVALFNQLYPQGLPQTWGGHGPEEVNGVRYYSWSGTLQPGKTDRGGNLFDGTNRSCRLFAKTFVREPGQCDGMVGRYSSHLGTVIGDDYPMDHFDIVNQSLGLVGKGADPVRLFVEHAARLKAAGV

SEQ ID NO: 35 - protein, 307 aa, Pseudomonas fluorescens
MLTPLDHKGVAMSQRCATRYPLVLVPGMLGFIRLVLYPYWYGIVSALRRGGAVVVAVKVSPLHSSEVRGEQLLARIEEILRQTGAQKVNLIGHSQGSLTARYAAAKRPDLVASVTSVAGTNHGSELADYLQLHYPADSAKGRVLSAVLRLINALMSLLDTGYRGPKLPVDVHASHASLTTAGVGVFNQRYPQGLPVTWGGNGPEEVNGVRYYSWSGTLQPGKTDKGRNLFDGTNRSCRLFARTFVREAGQCDGMVGRYSSHLGTVIGDDYPLDHFDIVNQSLGLVGKGAEPIRLFVEHAERLKAAGV

SEQ ID NO: 36 - protein, 296 aa, Pseudomonas sp.
MSQGSAPRYPLVLVPGMLGFIRLVLYPYWYGIVEVLRHGGAVVFAVQVSPLNSNEVRGEQLLARIDEILRETGADKVNLIGHSQGSLTARYAAAKRPDLVASVTSVAGPNHGSELADYLHQHYPHDSLRGRLLCFLLRMIGTLMGWLDTGYHGPKLPVDIHASHHALTTAGVALFNQRYPQGLPETWGGHGPEEVNGVRYYSWSGTLQPGITDRGRNLLDGTNRSCRLFAKTFVREAGHCDGMVGRYSSHLGTVIGDEYPLDHFDIVNQSLGLVGKGAEPIRLFVEHAARLSATGL

SEQ ID NO: 37 - protein, 277 aa, Pseudomonas fragi
MDDSVNTRYPILLVHGLFGFDRIGSHHYFHGIKQALNECGASVFVPIISAANDNEARGDQLLKQIHNLRRQVGAQRVNLIGHSQGALTARYVAAIAPELIASVTSVSGPNHGSELADRLRLAFVPGRLGETVAAALTTSFSAFLSALSGHPRLPQNALNALNALTTDGVAAFNRQYPQGLPDRWGGMGPAQVNAVHYYSWSGIIKGSRLAESLNLLDPLHNALRVFDSFFTRETRENDGMVGRFSSHLGQVIRSDYPLDHLDTINHMARGSAGASTR

SEQ ID NO: 38 - protein, 293 aa, Pseudomonas fragi
MDDSVNTRYPILLVHGLFGFDRIGSHHYFHGIKQALNECGASVFVPIISAANDNEARGDQLLKQIHNLRRQVGAQRVNLIGHSQGALTARYVAAIAPELIASVTSVSGPNHGSELADRLRLAFVPGRLGETVAAALTTSFSAFLSALSGHPRLPQNALNALNALTTDGVAAFNRQYPQGLPDRWGGMGPAQVNAVHYYSWSGIIKGSRLAESLNLLDPLHNALRVFDSFFTRETRENDGMVGRFSSHLGQVIRSDYPLDHLDTINHMARGSRRRINPVELYIEHAKRLKEAGL

SEQ ID NO: 39 - DNA, 960 bp, Burkholderia cepacia
  source 1..960: mol_type = genomic DNA
gccgacaactacgcggcgacgcgttatccgatcattctcgtgcacgggctcacgggcaccgacaaatacgcaggtgtgctcgagtactggtacgggatccaggaggacctgcagcagcgtggcgcgaccgtctatgtcgctaacctgtcgggcttccagagcgacgacggcccgaacgggcgcggcgaacagttgctggcctacgtgaagacggtgctcgccgcgacgggggcgaccaaggtcaacctcgtcggccacagccagggcgggctgacgtcgcgctatgtcgcggccgtcgcgcccgatctggtcgcgtcggtgacgacgatcggcacgccgcatcgcggctccgagttcgccgacttcgtgcagggcgtgctcgcgtacgatccgaccgggctgtcgtcgacggtgatcgccgcgttcgtcaatgtgttcggaatcctcacgagcagcagcaacaacacgaaccaggacgcgctcgcggcgctgaagacgctgacgaccgcgcaggccgccacgtacaaccagaactaccctagcgcgggcctcggcgcgccgggcagttgccagaccggcgcgccgacggaaaccgtcggcggcaacacgcatctgctgtattcgtgggccggcacggcgatccagccgacgatctccgtgttcggcgtcacgggtgcgacggatacgagcaccattccgctcgtcgatccggcgaacgcgctcgacccgtcgacgctcgcgctgttcggcaccggcacggtgatggtcaaccgcggttcgggccagaacgacggggtcgtgtcgaagtgcagcgcgctgtacggccaggtgctgagcacgagctacaagtggaaccatctcgacgagatcaaccagttgctcggcgtgcgcggcgcgaatgcggaagatccggtcgcggtgatccgcacgcatgcgaaccggctgaagctcgcgggcgtg

SEQ ID NO: 40 - protein, 320 aa, Pseudomonas luteola
ADNYATTRYPIILVHGLTGTDKYAGVLEYWYGIQEDLQQHGATVYVANLSGFQSDDGPKGRGEQLLAYVKTVLAATGATKVNLVGHSQGGLTSRYVAAVAPQLVASVTTIGTADRGSEFADFVQGVLAYDPTGLSSSVIAAFVNVFGILTSSSHNANQDALAGLKTLTTAQAATYNQNYPSAGLGRPGSCQTGAPTETVGGNTHLLYSWAGTAIQPTISVFGVTGATDTSTIPLIDPANVLDPSTLALFGTGTVMINRASGQNDGLVSKCSALYGKVLSTSYKWNHLDEINQLLGVRGANAEDPVAVIRTHANRLKLAGV

SEQ ID NO: 41 - protein, 311 aa, Pseudomonas stutzeri
MNKNKTLLALCLGSALALSGQAFAATGSGYTATKYPIVLAHGMLGFDSLLGIDYWYGIPSALRRDGAQVYVTEVSQLNTSELRGEELLAQVEEIVAISGKPKVNLIGHSHGGPTIRYVAGVRPDLIASVTSVGAPHKGSDVADLIRKVPEGSSGEAIIAGLVNAMGAFINFVSGSSSTAPQNSLGSLESLNSEGAARFNAKFPQGIPTTACGEGAYKVNGVHYYSWSGTSPLTNPLDVSDAMMGAGSLAFSGPNDGLVGRCSSHLGMVIRDNYRMNHLDEVNQFMGLTSLFETDPVSVYRQHANRLKNAGL

SEQ ID NO: 42 - protein, 311 aa, Pseudomonas mendocina
MNKNKTLLALCLGSALALSGQAFAATGSGYTATKYPIVLTHGMLGFDSLLGIDYWYGIPSALRRDGAQVYITEVSQLNTSELRGEELLAQVEEIVAISGKPKVNLIGHSHGGPTIRYVAGVRPDLIASVTSVGAPHKGSDVADLIRKVPEGSSGEAIIAGLVNAMGAFINFVSGSSSTAPQNSLGSLESLNSEGAARFNAKFPQGIPTTACGEGAYKVNGVRYYSWSGTSPLTNPLDVSDAMMGAGSLAFSGPNDGLVGRCSSHLGMVIRDDYRMNHLDEVNQFMGLTSLFETDPVSVYRQHANRLKNAGL

(19) United States
(12) Patent Application Publication
(10) Pub. No.: US 2024/0392270 A1
(43) Pub. Date: Nov. 28, 2024

(54) ENGINEERED LIPASE ENZYMES, MANUFACTURE AND USE THEREOF

(71) Applicant: Anagram Therapeutics, Inc., Framingham, MA (US)

(72) Inventors: Robert Gallotto, Medway, MA (US); Alexey Margolin, Newton, MA (US); Hugh Wight, Sudbury, MA (US); Jack Greene, Allston, MA (US); Mark Welch, Fremont, CA (US); Claes Gustafsson, Belmont, CA (US); Sridhar Govindarajan, Los Altos, CA (US)

(21) Appl. No.: 18/697,203

(22) PCT Filed: Sep. 30, 2022
(86) PCT No.: PCT/US2022/077426

§ 371 Date: Mar. 29, 2024

Related U.S. Application Data
(60) Provisional application No. 63/250,403, filed on Sep. 30, 2021.

(51) Int. Cl. C12N 9/20; A61P 3/00

(52) U.S. Cl. CPC C12N 9/20; A61P 3/00; C12Y 301/01003

(57) ABSTRACT

Provided are engineered lipase enzymes, methods of making such engineered lipases, dosage forms containing such engineered lipases and, methods of using such engineered lipases for treating diseases or disorders associated with reduced ability to digest and/or absorb triglycerides (fats).

### CROSS-REFERENCE TO RELATED APPLICATIONS

This application claims the benefit of and priority to co-pending U.S. Provisional Patent Application No. 63/250,403, filed Sep. 30, 2021, the entire contents of which are incorporated by reference herein.

### FIELD OF THE INVENTION

The invention relates generally to engineered lipase enzymes, methods of making such engineered lipases, dosage forms containing such engineered lipases and, methods of using such engineered lipases for treating diseases or disorders associated with reduced ability to digest and/or absorb triglycerides (fats).

### BACKGROUND

Long-chain triglycerides (fats) are the most abundant and important source of dietary lipids. Their digestion and absorption depends on an intricate interplay among pancreatic lipase, colipase, bile acids, transit time through the body, site of absorption, and meal content. Pancreatic lipase hydrolyzes triglyceride molecules to generate two fatty acid molecules and a 2-monoacylglycerol molecule. For this, lipase binds to the oil-water interface of triglyceride containing droplets. Once liberated, the long-chain free fatty acids and 2-monoacylglycerol molecules are absorbed in the small intestine and transported to the plasma and tissues and also used as energy. As such, there is a limited time for the lipase to digest fats to facilitate absorption in the small intestine.

Although humans typically produce a sufficient supply of pancreatic lipase to digest triglycerides, there are certain diseases and disorders that significantly impact this intricate physiological balance. Malabsorption syndrome is a series of life-threatening conditions that impact one or more of the steps in the hydrolysis or absorption of triglycerides. Malabsorption of fat can be caused by (1) an impaired secretion of pancreatic enzymes usually associated with exocrine pancreatic insufficiency (EPI), (2) amelioration in gastric, duodenal, liver, bile or gallbladder physiology, exhibited as (a) altered gastric secretion, (b) disturbed gastrointestinal transit, motility, mixing, emptying and/or (c) critical loss of intestinal mucosa function due to mucosal damage. Diseases that affect the pancreas such as cystic fibrosis (CF), chronic pancreatitis (CP), and pancreatic cancer can result in malabsorption of fats leading to malnutrition.

The current standard of care uses porcine-derived products (PERTs), e.g., pancrelipase, and pancreatin, which are known to have several limitations, including loss of activity from acid denaturation and proteolytic degradation during transit in the gastrointestinal (GI) tract (Lankisch et al. (1993) DIGESTION 54:148-155; Thiruvengadam et al. EP (1988) AM. J. PHYSIOL. 255:G476-G481; Guarner et al. (1993) GUT 34:708-712). Porcine-derived enzymes are extracted from pig pancreas in slaughterhouses and can contain certain impurities, including poorly characterized proteins, porcine viruses and other biological substances. Porcine extract (e.g., pancrelipase) based standard of care products have a significant limitation in that the lipase is inactivated by the low pH of the stomach and by proteolytic degradation (DiMagno et al. (1977) N. ENGL. J. MED. 296(23):1318-22.) To prevent inactivation, the current preparations often are enteric coated using phthalates, which prevents the release of the contents of the preparations until the pH reaches 5.5 (Creon®, Prescribing Information, Pharmacokinetics; Katherine E. Kelley et al. (2012) ENVIRON. HEALTH PERSPECT. 120(3): 379-384). There have been numerous attempts to use bacterial or fungal lipases to treat fat malabsorption that have failed due to the intricate nature of fat digestion and absorption as well as the inherent instability of the lipases due to acid denaturation, proteolytic degradation or unfolding and bile salt inhibition. Manufacturers attempts to improve stability (survivability) with enteric coating or chemical stabilization technologies have led to a mismatch in the availability of lipase and lipid substrate required for proper fat digestion and absorption in the small intestine. Additionally, in spite of chronic use, current PERTs are ineffective as clinical nutrition goals are not being met especially in adults and younger children with cystic fibrosis (CF). Poor hydrolysis of current PERTs can result in reduced caloric intake, poor weight management and significant levels of undesirable GI symptoms dramatically impacting quality of life. Furthermore, due to the poor stability of lipase in PERTs there is no liquid compatible formulation available for infants, children or adults unable to swallow pills.

A stable lipase that can be immediately active without the need for enteric coating or other technologies that could interfere with solubility provides the potential to have a longer period of time where the lipase can interact with the fat substrate allowing for additional substrate hydrolysis and absorption. In people with EPI, pancreatic and duodenal bicarbonate secretion is insufficient to neutralize the gastric acid load. Hence, the duodenal pH typically is lower in subjects with CF compared with healthy subjects. Accordingly, CF patients can have significantly longer postprandial periods in which the duodenal pH is below 4. As a result, this prolonged period of time when hyperacidity exists pushes out the time at which the lipase enzymes in the subject become available for digesting fats and can delay fat digestion and absorption, potentially missing significant portions of the duodenum which leads to steatorrhea and significant undesirable GI symptoms. The small bowel buffering capacity delay in EPI subjects appears to support the concept that delayed dissolution of enteric coated products due to poor solubility can be a factor in poor fat absorption (Gelfond et al. (2017) CLINICAL AND TRANSLATIONAL GASTROENTEROLOGY (2017) 8, e81).

While absorption of long-chain triglycerides first requires the enzymatic action of pancreatic lipases, medium chain triglycerides, due to their shorter chain lengths, can be absorbed across the intestinal lumen with the action of gastric lipase. While all fats provide caloric benefit, they have different impacts on physiological functions (St-Ogne et al. (2002) JOURNAL OF NUTRITION 132(3):329-332). While both long-chain triglycerides and medium chain triglycerides provide calories, only long-chain triglycerides in the form of long-chain polyunsaturated fatty acids (e.g., docosahexaenoic acid (DHA) and eicosapentaenoic acid (EPA)) provide structural components of membranes and biological mediators involved in the regulation of many physiological functions. Further medium chain triglycerides, when substituted for long-chain triglycerides, have been shown to increase energy expenditure and satiety, leading to reduced overall caloric intake and reduced body fat mass. As such, proper digestion and absorption of long-chain lipids is critical for good health.

Despite the efforts that have been made to date in treating disorders associated with reduced ability to digest and/or absorb triglycerides (fats), there is still an ongoing need for new and effective therapies for treating such disorders.

### SUMMARY OF THE INVENTION

The present invention is based, in part, upon the development of engineered lipase enzymes optimized to provide enhanced activity in the gastrointestinal tract, as well as reduced sensitivity to proteolytic degradation and increased tolerance to acidic pH levels. The engineered lipase enzymes can hydrolyze physiologically relevant fats (triglycerides) at the pH range early in the digestion process, for example, during transport through the stomach where a low pH environment exists (e.g., in the range of 60 to 120 minutes), which then facilitates the rapid absorption of resulting fatty acids during migration through the small intestine over a brief period of time, e.g., in the range of 2 to 4 hours. Furthermore, it is contemplated that the recombinant enzymes described herein, given their enhanced survivability, may be suitable for oral administration, and therefore potentially safer and more tolerable than the commercially available PERT enzymes. The terms “stability” and “survivability” are used interchangeably herein to refer to the ability of a lipase to maintain a functional activity, e.g., enzymatic activity, under predetermined conditions, e.g., under conditions encountered in the gastrointestinal tract of a primate subject. Measuring stability/survivability can be done using any method described herein, including for example, assessing the ability of a lipase to break down a lipid triglyceride into a monoglyceride and free fatty acids. The engineered lipase enzymes can be used for treating diseases or disorders associated with a reduced ability to digest or absorb fats (triglycerides).

In one aspect, the disclosure relates to a recombinant mutant microbial lipase enzyme (e.g., a mutant Burkholderia cepacia lipase), wherein the lipase comprises one or more of the following features, (i) increased stability at acidic pH (e.g., pH 3.0 or 4.0) relative to a corresponding wild-type microbial lipase enzyme, (ii) increased stability in the presence of a protease (e.g., a serine protease and/or an aspartic protease) relative to the corresponding wild-type microbial lipase enzyme, or (iii) at least 60%, 70%, 80%, 85%, 90%, 95%, 96%, 97%, 98%, 99%, or 100% of the enzymatic activity of the corresponding wild-type microbial lipase enzyme, including, for example, features (i), (ii), (iii), (i) and (ii), (i) and (iii), (ii) and (iii), and (i), (ii) and (iii).

In certain embodiments, the lipase comprises:
(a) a substitution of a residue at a position corresponding to position 39 of wild-type Burkholderia cepacia lipase;
(b) a substitution of a residue at a position corresponding to position 79 of wild-type B. cepacia lipase;
(c) a substitution of a residue at a position corresponding to position 102 of wild-type B. cepacia lipase;
(d) a substitution of a residue at a position corresponding to position 125 of wild-type B. cepacia lipase;
(e) a substitution of a residue at a position corresponding to position 128 of wild-type B. cepacia lipase;
(f) a substitution of a residue at a position corresponding to position 137 of wild-type B. cepacia lipase;
(g) a substitution of a residue at a position corresponding to position 138 of wild-type B. cepacia lipase;
(h) a substitution of a residue at a position corresponding to position 153 of wild-type B. cepacia lipase;
(i) a substitution of a residue at a position corresponding to position 154 of wild-type B. cepacia lipase;
(j) a substitution of a residue at a position corresponding to position 161 of wild-type B. cepacia lipase;
(k) a substitution of a residue at a position corresponding to position 170 of wild-type B. cepacia lipase;
(l) a substitution of a residue at a position corresponding to position 221 of wild-type B. cepacia lipase;
(m) a substitution of a residue at a position corresponding to position 227 of wild-type B. cepacia lipase;
(n) a substitution of a residue at a position corresponding to position 240 of wild-type B. cepacia lipase;
(o) a substitution of a residue at a position corresponding to position 249 of wild-type B. cepacia lipase;
(p) a substitution of a residue at a position corresponding to position 250 of wild-type B. cepacia lipase;
(q) a substitution of a residue at a position corresponding to position 260 of wild-type B. cepacia lipase;
(r) a substitution of a residue at a position corresponding to position 266 of wild-type B. cepacia lipase;
(s) a substitution of a residue at a position corresponding to position 281 of wild-type B. cepacia lipase;
(t) a substitution of a residue at a position corresponding to position 300 of wild-type B. cepacia lipase;
   or a combination of any of the foregoing substitutions.

In certain embodiments:
(a) the residue at a position corresponding to position 39 of wild-type B. cepacia lipase is substituted by R;
(b) the residue at a position corresponding to position 79 of wild-type B. cepacia lipase is substituted by Q;
(c) the residue at a position corresponding to position 102 of wild-type B. cepacia lipase is substituted by Q;
(d) the residue at a position corresponding to position 125 of wild-type B. cepacia lipase is substituted by S;
(e) the residue at a position corresponding to position 128 of wild-type B. cepacia lipase is substituted by N;
(f) the residue at a position corresponding to position 137 of wild-type B. cepacia lipase is substituted by A;
(g) the residue at a position corresponding to position 138 of wild-type B. cepacia lipase is substituted by I;
(h) the residue at a position corresponding to position 153 of wild-type B. cepacia lipase is substituted by N;
(i) the residue at a position corresponding to position 154 of wild-type B. cepacia lipase is substituted by H;
(j) the residue at a position corresponding to position 161 of wild-type B. cepacia lipase is substituted by A;
(k) the residue at a position corresponding to position 170 of wild-type B. cepacia lipase is substituted by S;
(l) the residue at a position corresponding to position 221 of wild-type B. cepacia lipase is substituted by L;
(m) the residue at a position corresponding to position 227 of wild-type B. cepacia lipase is substituted by K;
(n) the residue at a position corresponding to position 240 of wild-type B. cepacia lipase is substituted by V;
(o) the residue at a position corresponding to position 249 of wild-type B. cepacia lipase is substituted by L;
(p) the residue at a position corresponding to position 250 of wild-type B. cepacia lipase is substituted by A;
(q) the residue at a position corresponding to position 260 of wild-type B. cepacia lipase is substituted by A;
(r) the residue at a position corresponding to position 266 of wild-type B. cepacia lipase is substituted by L;
(s) the residue at a position corresponding to position 281 of wild-type B. cepacia lipase is substituted by A;
(t) the residue at a position corresponding to position 300 of wild-type B. cepacia lipase is substituted by Y;
   or the lipase comprises a combination of any of the foregoing substitutions.

In certain embodiments, the lipase comprises:
(a) a substitution of a Q residue at a position corresponding to position 39 of wild-type B. cepacia lipase (Q39);
(b) a substitution of a T residue at a position corresponding to position 79 of wild-type B. cepacia lipase (T79);
(c) a substitution of a D residue at a position corresponding to position 102 of wild-type B. cepacia lipase (D102);
(d) a substitution of a G residue at a position corresponding to position 125 of wild-type B. cepacia lipase (G125);
(e) a substitution of an A residue at a position corresponding to position 128 of wild-type B. cepacia lipase (A128);
(f) a substitution of a T residue at a position corresponding to position 137 of wild-type B. cepacia lipase (T137);
(g) a substitution of a V residue at a position corresponding to position 138 of wild-type B. cepacia lipase (V138);
(h) a substitution of an S residue at a position corresponding to position 153 of wild-type B. cepacia lipase (S153);
(i) a substitution of a N residue at a position corresponding to position 154 of wild-type B. cepacia lipase (N154);
(j) a substitution of an L residue at a position corresponding to position 161 of wild-type B. cepacia lipase (L161);
(k) a substitution of an A residue at a position corresponding to position 170 of wild-type B. cepacia lipase (A170);
(l) a substitution of an F residue at a position corresponding to position 221 of wild-type B. cepacia lipase (F221);
(m) a substitution of a T residue at a position corresponding to position 227 of wild-type B. cepacia lipase (T227);
(n) a substitution of an A residue at a position corresponding to position 240 of wild-type B. cepacia lipase (A240);
(o) a substitution of an F residue at a position corresponding to position 249 of wild-type B. cepacia lipase (F249);
(p) a substitution of a G residue at a position corresponding to position 250 of wild-type B. cepacia lipase (G250);
(q) a substitution of an S residue at a position corresponding to position 260 of wild-type B. cepacia lipase (5260);
(r) a substitution of a V residue at a position corresponding to position 266 of wild-type B. cepacia lipase (V266);
(s) a substitution of an S residue at a position corresponding to position 281 of wild-type B. cepacia lipase (S281);
(t) a substitution of an N residue at a position corresponding to position 300 of wild-type B. cepacia lipase (N300);
   or a combination of any of the foregoing substitutions.

In certain embodiments:
(a) the Q residue at a position corresponding to position 39 of wild-type B. cepacia lipase is substituted by R (Q39R);
(b) the T residue at a position corresponding to position 79 of wild-type B. cepacia lipase is substituted by Q (T79Q);
(c) the D residue at a position corresponding to position 102 of wild-type B. cepacia lipase is substituted by Q (D102Q);
(d) the G residue at a position corresponding to position 125 of wild-type B. cepacia lipase is substituted by S (G125S);
(e) the A residue at a position corresponding to position 128 of wild-type B. cepacia lipase is substituted by N (A128N);
(f) the T residue at a position corresponding to position 137 of wild-type B. cepacia lipase is substituted by A (T137A);
(g) the V residue at a position corresponding to position 138 of wild-type B. cepacia lipase is substituted by I (V1381);
(h) the S residue at a position corresponding to position 153 of wild-type B. cepacia lipase is substituted by N (S153N);
(i) the N residue at a position corresponding to position 154 of wild-type B. cepacia lipase is substituted by H (N154H);
(j) the L residue at a position corresponding to position 161 of wild-type B. cepacia lipase is substituted by A (L161A);
(k) the A residue at a position corresponding to position 170 of wild-type B. cepacia lipase is substituted by S (A170S);
(l) the F residue at a position corresponding to position 221 of wild-type B. cepacia lipase is substituted by L (F221L);
(m) the T residue at a position corresponding to position 227 of wild-type B. cepacia lipase is substituted by K (T227K);
(n) the A residue at a position corresponding to position 240 of wild-type B. cepacia lipase is substituted by V (A240V);
(o) the F residue at a position corresponding to position 249 of wild-type B. cepacia lipase is substituted by L (F249L);
(p) the G residue at a position corresponding to position 250 of wild-type B. cepacia lipase is substituted by A (G250A);
(q) the S residue at a position corresponding to position 260 of wild-type B. cepacia lipase is substituted by A (S260A);
(r) the V residue at a position corresponding to position 266 of wild-type B. cepacia lipase is substituted by L (V266L);
(s) the S residue at a position corresponding to position 281 of wild-type B. cepacia lipase is substituted by A (S281A);
(t) the N residue at a position corresponding to position 300 of wild-type B. cepacia lipase is substituted by Y (N300Y);
   or the lipase comprises a combination of any of the foregoing substitutions.

In certain embodiments, the lipase comprises a plurality of substitutions, for example 2, 3, 4, 5, 6, 7, 8, 9, 10, 11, 12, 13, 14, 15 or more different substitutions. For example, the lipase may contain 3 substitutions. Alternatively, the lipase may contain 4 substitutions. Alternatively, the lipase may contain 5 substitutions. Alternatively, the lipase may contain 6 substitutions. Alternatively, the lipase may contain 7 substitutions. Alternatively, the lipase may contain 8 substitutions. Alternatively, the lipase may contain 9 substitutions. Alternatively, the lipase may contain 10 substitutions. Alternatively, the lipase may contain 11 substitutions. Alternatively, the lipase may contain 12 substitutions.

In certain embodiments, the lipase comprises:
(a) the D102Q, N154H, and F221L substitutions;
(b) the D102Q, G125S, N154H, F221L, V266L, and N300Y substitutions;
(c) the T79Q, D102Q, G125S, T137A, N154H, F221L, T227K, F249L, V266L, and N300Y substitutions;
(d) the T79Q, D102Q, G125S, T137A, N154H, F221L, T227K, V266L, S281A, and N300Y substitutions;
(e) the T79Q, D102Q, G125S, S153N, N154H, F221L, T227K, V266L, S281A, and N300Y substitutions;
(f) the T79Q, D102Q, G125S, S153N, N154H, F221L, F249L, G250A, V266L, and N300Y substitutions;
(g) the T79Q, D102Q, G125S, S153N, N154H, F221L, F249L, V266L, S281A, and N300Y substitutions;
(h) the T79Q, D102Q, G125S, N154H, F221L, T227K, F249L, V266L, S281A, and N300Y substitutions;
(i) the D102Q, G125S, T137A, S153N, N154H, F221L, T227K, F249L, V266L, and N300Y substitutions;
(j) the D102Q, G125S, T137A, S153N, N154H, F221L, T227K, G250A, V266L, and N300Y substitutions;
(k) the D102Q, G125S, T137A, N154H, F221L, T227K, G250A, V266L, S281A, and N300Y substitutions;
(l) the D102Q, G125S, S153N, N154H, F221L, T227K, F249L, G250A, V266L, and N300Y substitutions; or
(m) the D102Q, G125S, S153N, N154H, F221L, T227K, F249L, V266L, S281A, and N300Y substitutions.

In certain embodiments, the lipase is a α/β-hydrolase lipase, the lipase may comprise an active site that contains a serine-histidine-aspartate triad. Alternatively or in addition, the lipase may comprise a hydrophobic lid that opens to allow for the binding and/or hydrolysis of a triglyceride having, for example, a chain length of greater than eight carbons. Alternatively or in addition, in certain embodiments, the lipase comprises a calcium binding site, wherein, when calcium is bound to the calcium binding site, the lipase is stabilized. Alternatively or in addition, in certain embodiments, the lipase comprises an oxyanion hole, wherein the oxyanion hole stabilizes a negatively charged intermediate generated during fatty acid bond hydrolysis.

In certain embodiments, the lipase is a fungal lipase or a bacterial lipase. In certain embodiments, the lipase is a Family I bacterial lipase, e.g., an I.1, I.2, or I.3 subfamily bacterial lipase, e.g., a I.1 or I.2 subfamily bacterial lipase. In certain embodiments, the lipase is a I.2 subfamily bacterial lipase.

In certain embodiments, the lipase is a Burkholderia, Pseudomonas, or Chromobacterium lipase. In certain embodiments, the lipase is a Burkholderia cepacia (B. cepacia), Burkholderia glumae, Pseudomonas fluorescens, Pseudomonas aeruginosa, Pseudomonas luteola, or Chromobacterium viscosum lipase. In certain embodiments, the lipase is a Burkholderia cepacia lipase.

In certain embodiments, the lipase comprises a S residue at a position corresponding to position 87 of wild-type B. cepacia (S87), a D residue at a position corresponding to position 264 of wild-type B. cepacia (D264), and a H residue at a position corresponding to position 286 of wild-type B. cepacia (H286). These amino acids are conserved between lipase subfamilies I.1 and I.2 (see, FIG. 3).

In certain embodiments, the lipase comprises the amino acid sequence of any one of SEQ ID NOs: 2-14, or an amino acid sequence that has at least 85%, 90%, 95%, 96%, 97%, 98%, or 99% sequence identity to any one of SEQ ID NOs: 2-14.

In certain embodiments, the lipase comprises one, two, three, four, five, six, seven, eight, nine, ten, or more than ten mutations relative to the corresponding wild-type microbial lipase.

In another aspect, the disclosure relates to a recombinant mutant microbial lipase enzyme comprising a substitution, or combination of substitutions, listed in TABLE 1 or TABLE 2.

In certain embodiments, the lipase disclosed herein has a half-life of at least 75 minutes, 100 minutes, 125 minutes, 150 minutes, 175 minutes, 180 minutes, 185 minutes, 190 minutes, 195 minutes, or 200 minutes in the presence of a serine protease. Alternatively or in addition, in certain embodiments, the lipase has at least 0.5 fold, 1 fold, 1.5 fold, 2 fold, 2.5 fold, or 3 fold higher stability in the presence of a serine protease (e.g., Aspergillus melleus protease), compared to the corresponding wild-type lipase.

In certain embodiments, the lipase has a half-life of at least 50 minutes, 75 minutes, 100 minutes, 125 minutes, 130 minutes, 135 minutes, 140 minutes, 145 minutes, or 150 minutes at about pH 3.0. Alternatively or in addition, in certain embodiments, the lipase has at least 1.5 fold, 2 fold, 2.5 fold, or 3 fold higher stability at about pH 3.0, compared to the corresponding wild-type lipase.

In certain embodiments, the lipase has a half-life of at least 75 minutes, 100 minutes, 125 minutes, 150 minutes, 175 minutes, 200 minutes, 225 minutes, 230 minutes, or 235 minutes in the presence of an aspartic protease (e.g., at pH 3.6). Alternatively or in addition, in, certain embodiments, the lipase has at least 1.5 fold, 2 fold, 2.5 fold, 3 fold, 3.5 fold, or 4 fold higher stability in the presence of an aspartic protease (e.g., at pH 3.6), compared to the corresponding wild-type lipase. In certain embodiments, the aspartic protease is pepsin.

In certain embodiments, the lipase has a half-life of at least 75 minutes, 100 minutes, 125 minutes, 150 minutes, 175 minutes, 180 minutes, 185 minutes, 190 minutes, 195 minutes, or 200 minutes in the presence of pancreatin. Alternatively or in addition, in certain embodiments, the lipase has at least 0.5 fold, 1 fold, 1.5 fold, 2 fold, 2.5 fold, or 3 fold higher stability in the presence of pancreatin, compared to the corresponding wild-type lipase. Alternatively or in addition, in certain embodiments, the lipase has at least 0.5 fold, 1 fold, 1.5 fold, 2 fold, 2.5 fold, or 3 fold higher activity at about pH 3.0, compared to the corresponding wild-type lipase.

In certain embodiments, the lipase has a specific activity at pH 3.0 of at least 300, 400, 500, 600, 700, 800, 900, or 1,000 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3). In certain embodiments, the lipase has a specific activity at pH 4.0, pH 5.0, or pH 6.0 of at least 600, 700, 800, 900, 1,000, 1,100, 1,200, 1,300, 1,400, 1,500, or 2,000 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3). In certain embodiments, the lipase has a specific activity at pH 7.0 of at least 1,000, 1,100, 1,200, 1,300, 1,400, 1,500, or 2,000 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3).

In certain embodiments, the lipase preferentially hydrolyzes the sn-1 and sn-3 positions on a triglyceride and/or the lipase enzymatic activity (e.g., specific activity) is not inhibited by bile salts and/or the lipase does not require a colipase. In certain embodiments, the lipase is not cross-linked and/or crystallized.

In certain embodiments, the lipase remains sufficiently active at a pH in the range of 3.5 to 7.0 to hydrolyze long-chain poly-unsaturated fats (LCPUFAs), e.g., DHA and EPA, or long-chain triglycerides, e.g., oleic acid or triolein, in the gastrointestinal tract of a subject. In certain embodiments, the lipase is at least 2 fold, 10 fold, 100 fold or 1000 fold more active than pancrelipase when tested under the same conditions.

In certain embodiments, more than 50%, 60%, 70%, 80%, or 90% of the lipase remains active in the fed-state stomach of a subject for 60-120 minutes. In certain embodiments, the lipase digests greater than 20%, 30%, 40%, or 50% of ingested fats in the stomach of a subject to fatty acids and monoglycerides.

In certain embodiments, more than 50%, 60%, 70%, 80%, or 90% of the lipase remains active through the small intestine of a subject for 240-360 minutes. In certain embodiments, the lipase digests greater than 50%, 60%, 70%, 80%, or 90% of ingested fats in the small intestine of a subject to fatty acids and monoglycerides.

In certain embodiments, the lipase increases absorption of long-chain unsaturated fatty acids in the plasma in a subject within 30 minutes, 45 minutes, 60 minutes, 90 minutes, or 120 minutes by more than 25%, 35%, 50%, 100%, or 200% relative to the same subject when that subject has not been administered the lipase, or relative to a similar subject that has not been administered the lipase. In certain embodiments, the lipase increases absorption of fat-soluble vitamins (e.g., vitamin A, vitamin D, vitamin E, vitamin K). In certain embodiments, the lipase increases absorption of choline.

In another aspect, the disclosure relates to a nucleic acid encoding a lipase as described herein.

In another aspect, the disclosure relates an expression vector comprising a nucleic acid sequence as described herein. In certain embodiments, the nucleic acid sequence encoding the recombinant mutant lipase is codon optimized for expression in a heterologous cell. In certain embodiments, the heterologous cell is a B. cepacia, Burkholderia glumae, Pseudomonas fluorescens, Chromobacterium viscosum, Pseudomonas luteola, Pseudomonas fragi, or Escherichia coli cell.

In another aspect, the disclosure relates to a cell comprising an expression vector as described herein. In certain embodiments, the cell is a B. cepacia, Burkholderia glumae, Pseudomonas fluorescens, Chromobacterium viscosum, Pseudomonas luteola, Pseudomonas fragi, or Escherichia coli cell.

In certain embodiments, the disclosure relates to a method of producing a recombinant mutant microbial lipase enzyme, the method comprising growing a cell as described herein under conditions so that the host cell expresses the recombinant mutant microbial lipase enzyme, and purifying the recombinant mutant microbial lipase enzyme.

In another aspect, the disclosure relates to a pharmaceutical composition comprising a lipase as described herein and a pharmaceutically acceptable carrier and/or an excipient. In certain embodiments, the pharmaceutical composition further comprising a microbial protease, and/or a microbial amylase. In certain embodiments, the protease is an A. melleus protease and/or the amylase is an Aspergillus oryzae amylase. In certain embodiments, the composition is formulated as an oral dosage form. In certain embodiments, the composition is a formulated as a powder, granulate, pellet, micropellet, liquid, or a tablet. In certain embodiments, the composition is encapsulated in a capsule or formulated as a tablet dosage form. In certain embodiments, the composition does not comprise an enteric coating.

In another embodiment, the disclosure relates to a method of treating a disease or disorder associated with a reduced ability to digest or absorb lipids, resulting in an elevated amount of undigested lipid, in a subject in need thereof, the method comprising administering to the subject an effective amount of a lipase or a pharmaceutical composition as described herein, thereby treating the disease or disorder in the subject.

In another embodiment, the disclosure relates to a method of treating maldigestion or malabsorption of lipids in a subject in need thereof, the method comprising administering to the subject an effective amount of a lipase or a pharmaceutical composition as described herein, thereby treating the disease or disorder in the subject.

In certain embodiments, the subject exhibits low level secretion of pancreatic enzymes or has a physiological condition that affects fat hydrolysis or fat absorption (e.g., reduced gastric, duodenal, liver, bile, or gallbladder function); reduced gastrointestinal transit, motility, mixing, emptying; or reduced intestinal mucosa function (e.g., induced by mucosal damage) that results in fat maldigestion or fat malabsorption or a fatty acid deficiency.

In certain embodiments, the maldigestion or malabsorption of lipids is associated with a disease or disorder selected from exocrine pancreatic insufficiency (EPI), malabsorption syndrome, cystic fibrosis, chronic pancreatitis, acute pancreatitis, Schwachman-Diamond syndrome, a fatty acid disorder, Familial lipoprotein lipase deficiency, Johanson-Blizzard syndrome, Zollinger-Ellison syndrome, Pearson marrow syndrome, short-bowel syndrome, liver disease, primary biliary atresia, cholestasis, celiac disease, fatty liver disease, pancreatitis, diabetes, aging, cancer of the pancreas, stomach, small intestine, colon, rectal/anal, liver, hepatic, gallbladder, or, esophagus, cachexia, or a gastrointestinal disorder (e.g., Crohn's disease, irritable bowel syndrome, or ulcerative colitis), surgical invention of the stomach, small intestine, liver, gallbladder or pancreas.

In another embodiment, the disclosure relates to a method of improving the absorption of fatty acids in a subject in need thereof, the method comprising administering to the subject an effective amount of a lipase or a pharmaceutical composition as described herein, thereby improving absorption of fatty acids in the subject.

In another embodiment, the disclosure relates to a method of increasing the amount of fatty acids in plasma, erythrocytes, or a tissue of a subject in need thereof, the method comprising administering to the subject an effective amount of a lipase or a pharmaceutical composition as described herein, thereby increasing the amount of fatty acids in the subject.

In another embodiment, the disclosure relates to a method of increasing the ratio of omega-3 to omega-6 fatty acids in plasma, erythrocytes, or a tissue of a subject in need thereof, the method comprising administering to the subject an effective amount of a lipase or a pharmaceutical composition as described herein, thereby increasing the amount of fatty acids in the subject.

In another embodiment, the disclosure relates to a method of reducing the amount of fatty acids in the stool of a subject in need thereof, the method comprising administering to the subject an effective amount of a lipase or a pharmaceutical composition as described herein, thereby reducing the amount of fatty acids in the stool of the subject.

In certain embodiments, the fatty acids are long-chain poly-unsaturated fatty acids (LCPUFAs). In certain embodiments, the fatty acids are omega-3 fatty acids. In certain embodiments, the omega-3 fatty acids are DHA, EPA, or DPA. In certain embodiments, the subject is administered less than 400, 600, 800, or 1,000 mg of the lipase or pharmaceutical composition per day. In certain embodiments, the lipase or pharmaceutical composition is administered in combination with a fat soluble vitamin (e.g., vitamin A, D, E, or K), an acid blocker, or a nutritional formula containing triglycerides.

In certain embodiments, the subject is a mammal, for example, a human.

These and other aspects and features of the invention are described in the following detailed description and claims.

### BRIEF DESCRIPTION OF THE DRAWINGS

The invention can be more completely understood with reference to the following drawings.

FIG. 1A schematically depicts an exemplary lipase, which, in the absence of long-chain triglycerides, is believed to exist in a closed conformation where the active site is protected from the environment due to interaction of the lid and subdomain, where the lid covers the active site cleft, and a subdomain covers the lid. It is believed that, in the presence of long-chain triglycerides, conformational changes in the lipase result in an open conformation where the lid and subdomain open to expose the active site cleft. Structural studies suggest that the hydrophobic lipid-binding site becomes exposed by the rolling back or opening movement of the lid from the active site at an oil-water interface.

FIG. 1B depicts a space filling model of the three-dimensional structure of an exemplary lipase from Burkholderia cepacia in both a closed, inactive conformation, and in an open, active conformation.

FIG. 2 depicts a ribbon model of a lipase from Burkholderia cepacia in which the amino acids 118-159 define the lid, amino acids 214-261 define the subdomain that faces the lid, residues 262-320 which includes a helix 11 and amino acids 160-213 which includes a helix 7. The amino acids that contribute to the catalytic triad (namely, serine 87, aspartic acid 264, and histidine 286) are depicted.

FIG. 3 illustrates a phylogenetic tree of Family I bacterial lipases and their classification into six subfamilies (referred to as I.1-I.6).

FIG. 4 depicts a sequence alignment showing the conservation of amino acids among the lipase sequences of Pseudomonas aeruginosa PAO1 (family I.1, SEQ ID NO: 29), Pseudomonas fluorescens (family I.1, SEQ ID NO: 30), Burkholderia cepacia (family I.2, SEQ ID NO: 1), Burkholderia glumae (family I.2, SEQ ID NO: 31), Chromobacterium viscosum (family I.2, SEQ ID NO: 32), Pseudomonas luteola (family I.2, SEQ ID NO: 33), Pseudomonas fluorescens ABA72135 (family I.1, SEQ ID NO: 34), Pseudomonas fluorescens AEV60646 (family I.1, SEQ ID NO: 35), Pseudomonas sp WP-015093259 (family I.3, SEQ ID NO: 36), Pseudomonas fragi CAA32193 (family I.1, SEQ ID NO: 37), Pseudomonas fragi CAC07191 (family I.1, SEQ ID NO: 38), Pseudomonas stutzeri (SEQ ID NO: 41) and Pseudomonas mendocina LipA (SEQ ID NO: 42). The amino acid residues that constitute the catalytic triad (active site) and calcium binding site, are depicted in the figure (boxed and shaded). Substitutions made in the final round of the lipase engineering (see, Example 7) are shown relative to the wild type B. cepacia sequence (box and no shading).

FIG. 5 depicts a sequence alignment showing the conservation of residues between Burkholderia cepacia (family I.2, SEQ ID NO: 1), Burkholderia glumae (family I.2, SEQ ID NO: 31), Chromobacterium viscosum (family I.2, SEQ ID NO:32), and Pseudomonas luteola (family I.2, SEQ ID NO: 40), where the conserved amino acids that constitute the oxyanion hole, the lid, the subdomain, the catalytic triad and calcium binding site are identified. The locations of amino acid substitutions made in the ultimate round of the lipase engineering (see, Example 7) relative to the wild type B. cepacia sequence are shown in boxes with dark outlines.

FIG. 6 depicts a three-dimensional model of a B. cepacia lipase showing the locations of the catalytic lid, the oxyanion hole, the catalytic triad, the calcium domains and the positions of the top variant substitutions.

FIG. 7 is a schematic of a B. cepacia lipase showing the locations of the active site/catalytic triad (stars), the calcium site (circles), the last round amino acid substitutions (triangles), the oxyanion hole, the lid, and the subdomain-facing lid (various shading).

FIG. 8 is a schematic for an exemplary three-step reaction for free fatty acid detection.

FIG. 9 is a flow diagram of the pH survivability assay. The lipase solution is pre-treated by incubation at specific pH for a series of timepoints, then assayed with 4-nitrophenyl palmitate (p-NPP) for lipase activity, the colorimetric response is detected at 405 nm and the pH stability over time for each pH is reported.

FIG. 10 illustrates the mechanism of p-NPP (colorless) hydrolysis into 4-nitrophenolate (pNP, yellow) by a lipase.

FIG. 11 is a flow diagram of the pepsin survivability assay. The lipase solution is pre-treated by incubation with pepsin for a series of timepoints, then assayed with 4-nitrophenyl palmitate (p-NPP) for lipase activity, the colorimetric response is detected at 405 nm and the half-life over time for pepsin is reported.

FIG. 12 is a flow diagram of the A. melleus protease (oryzin) survivability assay. The lipase solution is pre-treated by incubation with oryzin for a series of timepoints, then assayed with 4-nitrophenyl palmitate (p-NPP) for lipase activity, the colorimetric response is detected at 405 nm and the half-life over time for oryzin is reported.

FIG. 13 is a graph showing the impact of the indicated lipase mutations on stability in the presence of A. melleus protease, stability at low pH, stability in the presence of pepsin/SGF, activity at pH 4, and activity at pH 7 in the presence of bile salts.

FIG. 14 is a graph showing the stability or activity of the engineered mutants relative to the B. cepacia V290 lipase variant. Conditions tested were stability in the presence of A. melleus protease (t½), stability at low pH (t½), stability in the presence of pepsin/SGF (t½), and activity at pH 4 (U/mg).

FIG. 15 is a graph showing the half-life of the top 11 B. cepacia lipase variants at the conditions shown. Three controls were used: (1) wild-type (WT) B. cepacia lipase, (2) V130 (the top variant from an earlier round), and (3) V290 (the top variant from one of the later rounds).

FIG. 16 is a graph showing survivability improvement through lipase engineering for the top 3 B. cepacia lipase variants, V325, V366, and V318, at the conditions shown (proteolytic stability, stability at low pH, and stability in the presence of pepsin). Three controls were used: (1) wild-type (WT) B. cepacia lipase, (2) V130 variant, and (3) V290 variant. The Y-axis shows time in minutes.

FIG. 17 is a graph showing the percentage of lipase surviving A. melleus protease treatment at different timepoints (5, 30, 60, 120, 180, and 240 minutes). The graph shows the top 3 B. cepacia lipase variants, V325, V366, and V318 and three controls (wild-type (WT) B. cepacia lipase, V130 variant, V290 variant).

FIG. 18 is a graph showing the percentage of lipase surviving pH 3.0 treatment at different timepoints (5, 30, 60, and 120 minutes for the top 3 B. cepacia lipase variants, V325, V366, and V318, and three controls (wild-type (WT) B. cepacia lipase, V130 variant, V290 variant).

FIG. 19 is a graph showing the percentage of lipase surviving pepsin treatment at pH 3.58 (typical fed state stomach) at different timepoints (5, 30, 60, and 120 minutes) for the top 3 B. cepacia lipase variants V325, V366, and V318 and three controls (wild-type (WT) B. cepacia lipase, V130 variant, V290 variant).

FIGS. 20A and 20B is a graph showing the per meal activity (free fatty acid release DHA oil) of 40 mg (FIG. 20A) and 80 mg (FIG. 20B) of the wild-type lipase, the top three variants (V318, V325, and V336) and pancrelipase.

FIG. 21 is a schematic of treatment group design for a dose finding study for V325 in an EPI pig model as described in Experiment 1 of Example 9.

FIG. 22 is a graph showing the AUC and Cₘₐₓ for free fatty acids DHA and EPA in the plasma of animals administered an omega-3 triglyceride substrate and the indicated dosages of V325 or no enzyme (“NE”).

FIG. 23 is a graph showing the AUC₂₄ mean over time calculated from the AUC data provided in FIG. 22.

FIG. 24 is a graph showing baseline subtracted Cₘₐₓ calculated from the Cₘₐₓ data provided in FIG. 22.

FIG. 25 is a graph showing the AUC and Cₘₐₓ for total fatty acids in the plasma of animals administered a substrate and the indicated dosages of V325 or no enzyme (“NE”).

FIG. 26 is a graph showing the AUC₂₄ mean over time calculated from the AUC data provided in FIG. 25.

FIG. 27 is a graph showing baseline subtracted Cₘₐₓ calculated from the Cₘₐₓ data provided in FIG. 25.

FIG. 28 is a schematic of treatment group design for the evaluation of the activity and stability of V325 in an EPI pig model as described in Experiment 2 of Example 9.

FIG. 29A is a graph showing that the AUC and Cₘₐₓ for DHA+EPA in the plasma of animals administered an omega-3 triglyceride substrate and the indicated dosages of V325 were significantly higher than from animals administered Creon® or no enzyme (“NE”). FIG. 29B is a graph showing the AUC mean over time, baseline subtracted for 6, 8, 12, and 24 time points, calculated from the AUC data provided in FIG. 29A.

FIG. 30A is a graph showing that the AUC and Cₘₐₓ for total fatty acids in the plasma of animals administered a substrate and the indicated dosages of V325 were significantly higher than animals administered Creon® or no enzyme (“NE”). FIG. 30B is a graph showing the AUC mean over time, baseline subtracted for the 6, 8, 12, and 24 time points, calculated from the AUC data provided in FIG. 30A.

FIG. 31A is a graph showing the AUC for free fatty acid release over time in different compartments of the gastrointestinal tract (stomach, duodenum, ileum) for animals administered V325 or Creon®. FIG. 31B is a graph showing the AUC mean over time, calculated from the AUC data provided in FIG. 31A.

### DETAILED DESCRIPTION

The present invention is based, in part, upon the development of engineered lipase enzymes optimized to provide enhanced survivability and activity in the gastrointestinal tract, as well as reduced sensitivity to proteolytic degradation and increased tolerance to acidic pH levels. The engineered lipase enzymes can hydrolyze physiologically relevant fat triglycerides (long-chain poly-unsaturated fatty acids (LCPUFA) and dietary long-chain triglycerides) at the pH range early in the digestion process, e.g., during transport through the stomach where a low pH environment exists, which then facilitates the rapid absorption of resulting fatty acids during migration through the small intestine. Furthermore, it is contemplated that the recombinant enzymes described herein, given their enhanced stability, may be suitable for oral administration, and therefore potentially safer and more tolerable than the commercially available PERT enzymes. The engineered lipase enzymes can be used to treat diseases or disorders associated with a reduced ability to digest or absorb fats (triglycerides).

Various features and aspects of the invention are discussed in more detail below.

### I. Lipases

Typically, lipase enzymes hydrolyze dietary fats (triglycerides) to produce two fatty acid molecules and a monoacylglycerol molecule. Most lipases are members of the α/β hydrolase fold superfamily, one of the largest groups of structurally related yet functionally diverse enzymes. The three-dimensional structure of most lipases share a common fold motif, known as an α/β hydrolase fold.

Hydrolytic lipase enzymes that hydrolyze carboxy ester bonds in lipids, namely, carboxyesterases and true lipases are referred to collectively as lipolytic enzymes. Carboxyesterases (esterases) usually hydrolyze water-soluble esters, whereas true lipases (lipases) can also hydrolyze water insoluble substrates (Verger (1997) TRENDS IN BIOTECHNOLOGY 15(1):P32-38; Ali et al. (2012) Lipases and Phospholipases, New York, USA, Humana Press, p. 31-51). The longer the fatty acid chain lengths in a triglyceride the less water-soluble the triglycerides become. As a result, enzymes that hydrolyze long-chain triglycerides are called lipases and those that hydrolyze tributyrin (short chain C4 fatty acids) are called esterases (Jaeger et al. (1994) FEMS MICROBIOL REV 15:29-63). Long-chain triglycerides are predominately ingested in human foods whereas short-chain fatty acids typically are a bi-product of carbohydrate metabolism by anaerobic bacteria in the colon. A property of true lipases (also referred to herein as lipases) that distinguishes them from esterases is their enhanced activity at an oil-water interface, a phenomenon termed ‘interfacial activation’ (Schrag et al. (1991) NATURE 351(6329):761-764).

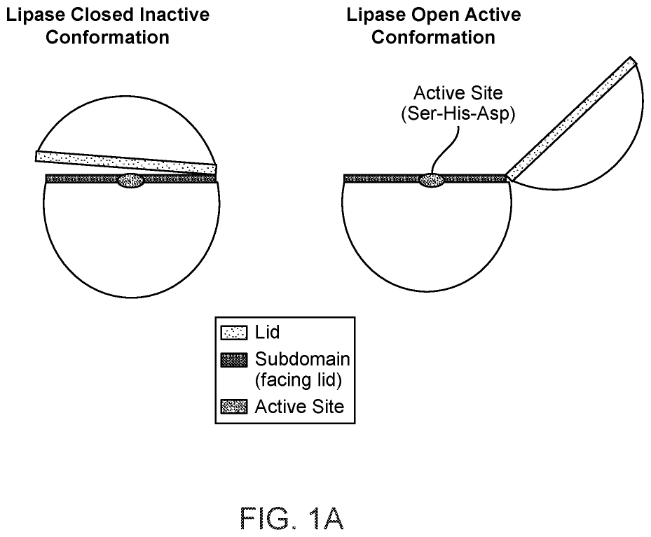

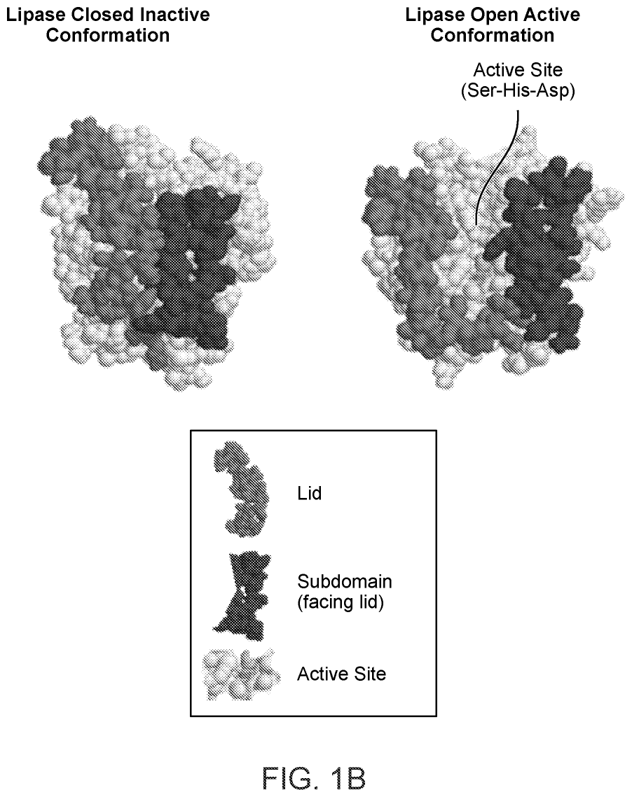

Lipases are structurally conserved and contain an active site cleft that, depending upon the surrounding conditions, is covered with a flexible and amphiphilic α-helix which functions as a “lid” to cover the active site cleft. If the lid is closed, the active site is protected from the environment and inaccessible to triglyceride substrates. FIG. 1A schematically depicts an exemplary lipase, which, in the absence of long-chain triglycerides, is believed to exist in a closed conformation where the active site is protected from the environment due to interaction of the lid and subdomain, where the lid covers the active site cleft, and a subdomain covers the lid. However, in the presence of long-chain triglycerides, conformational changes in the lipase result in an open conformation where the lid and subdomain open to expose the active site cleft. Structural studies suggest that the hydrophobic lipid-binding site becomes exposed by the rolling back or opening movement of the lid from the active site at an oil-water interface.

FIG. 1B depicts a space filling model of the three-dimensional structure of an exemplary lipase from Burkholderia cepacia in both an closed, inactive conformation, and in an open, active conformation. In the inactive conformation, the lid covers the active site. In the active conformation, the lid and subdomain (also referred to as a facing lid) move to expose the depicted active site cleft that contains three amino acid acids (a serine, histidine and an aspartic acid), which are conserved between many lipases (see, Brenner (1988) NATURE 334:528-530; Brady et al. (1990) NATURE 343(6260):767-70; Schrag et al. (1991), supra).

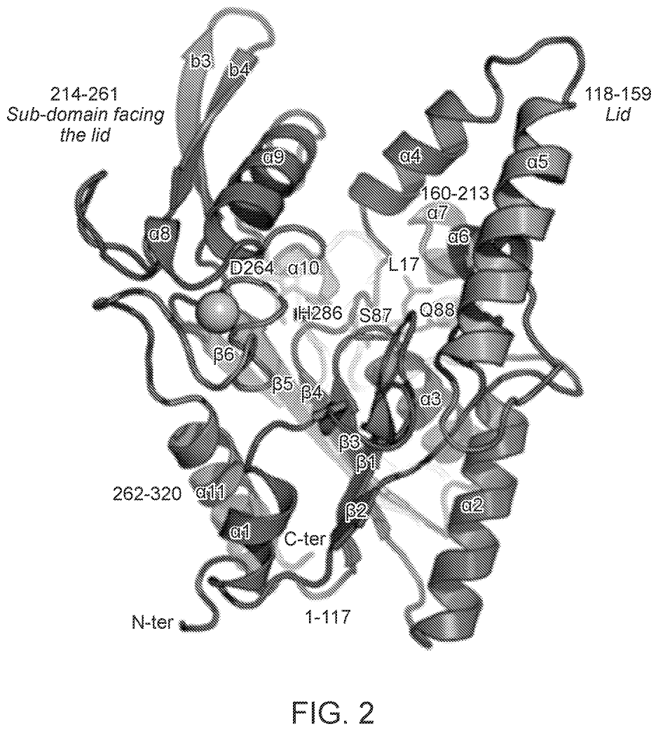

FIG. 2 depicts a ribbon model of a lipase from B. cepacia in which the amino acids 118-159 define the lid, amino acids 214-261 define the subdomain that faces the lid, residues 262-320 which includes α helix 11, and amino acids 160-213 which includes α helix 7. The amino acids that contribute to the catalytic triad (namely, serine 87, aspartic acid 264, and histidine 286) are depicted.

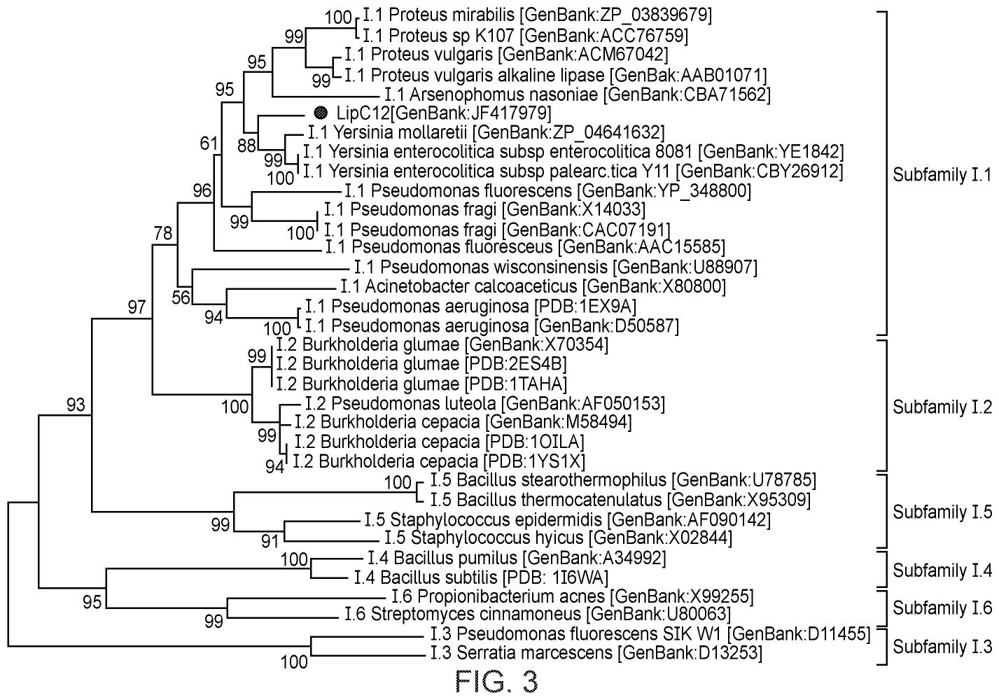

Bacterial lipases have been categorized into eight families (family I-VIII) based on differences in amino acid sequences and biological properties. Among them, family I, as depicted in FIG. 3, is the largest group and has been further subdivided into six subfamilies (referred to as I.1-I.6), of which families I.1, I.2, and I.3 are representative gram-negative bacterial lipases. The lipases in family I are highly conversed and the activities of this family of lipases rely on the presence of a catalytic active site formed by three conserved amino acids, namely serine, histidine and an aspartic acid. (Nardini et al. (2000) J. BIOL. CHEM. 275(40):31219-31225; Kim et al. (1997) STRUCTURE 5(2):173-185.)

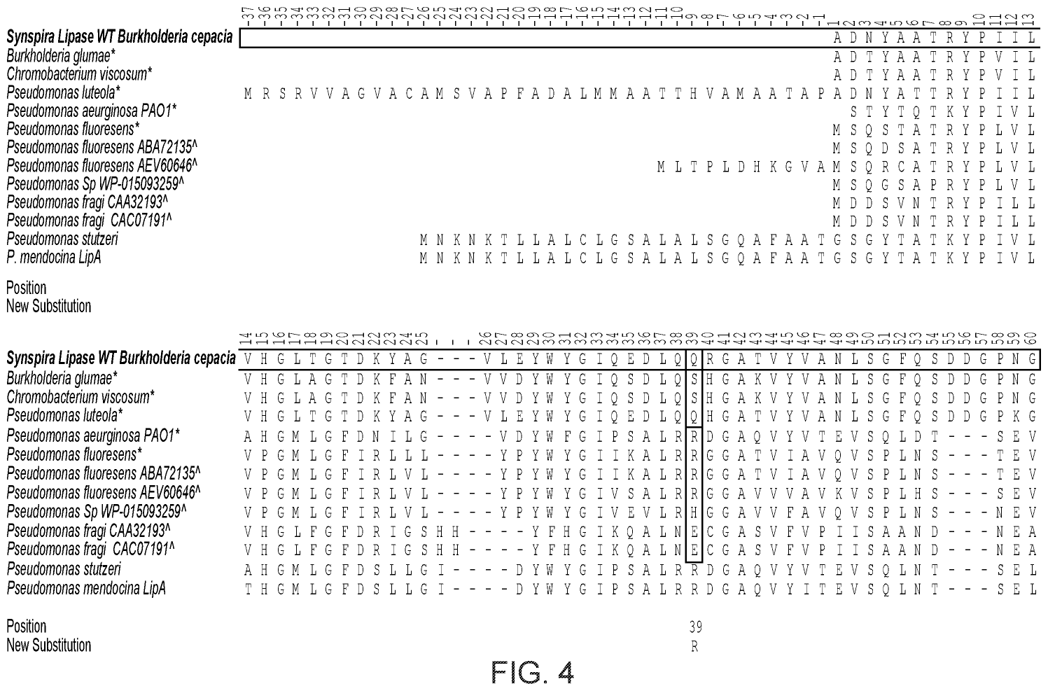

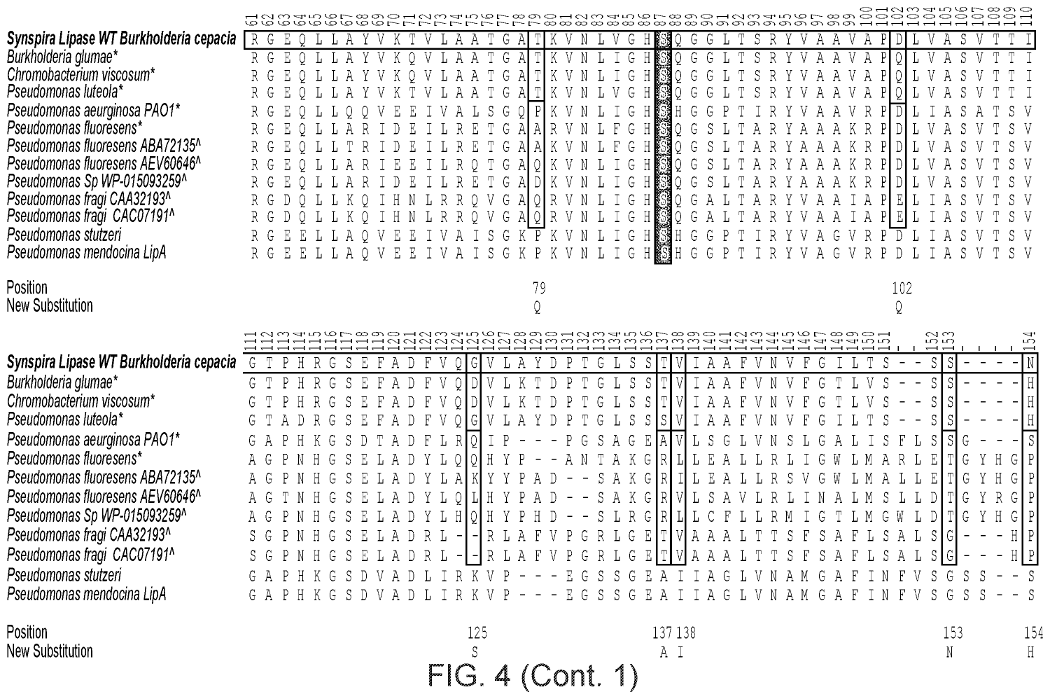

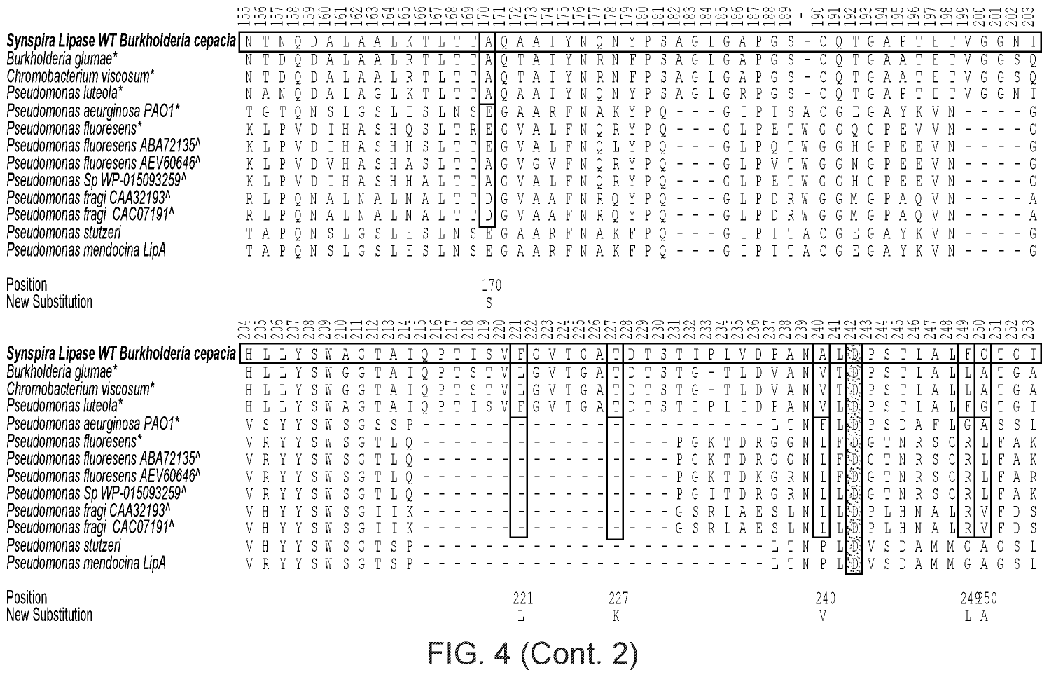

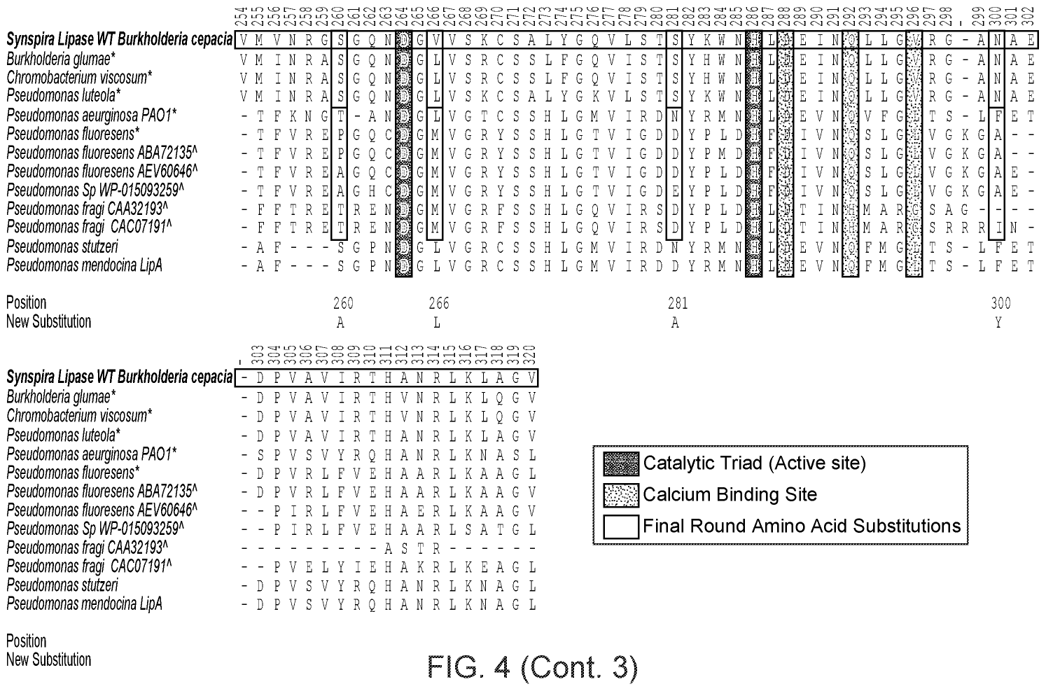

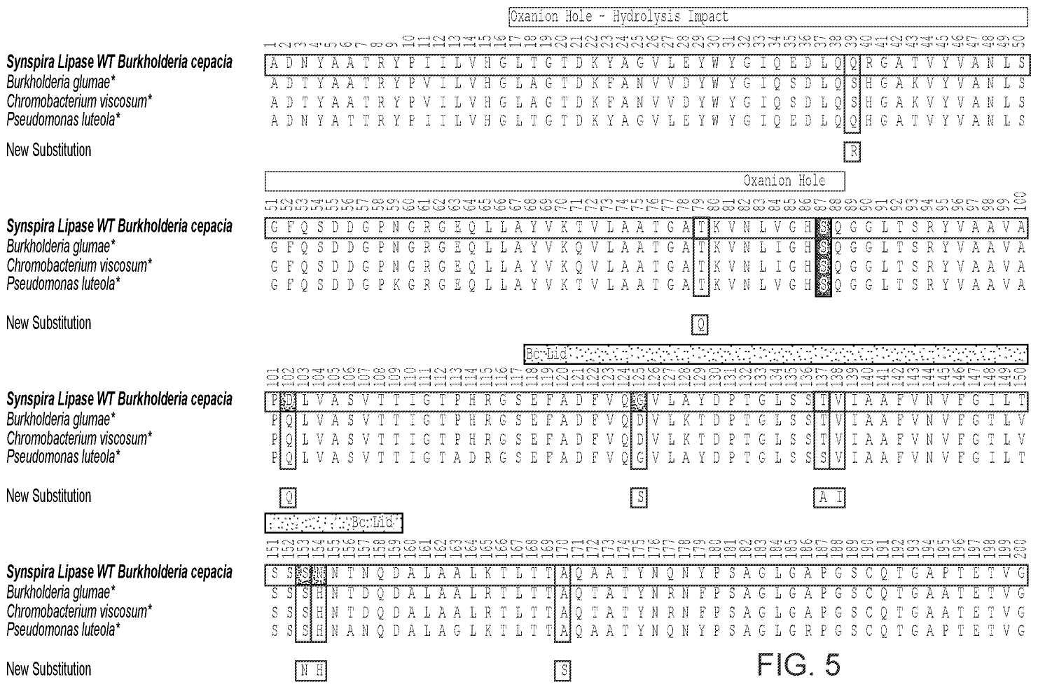

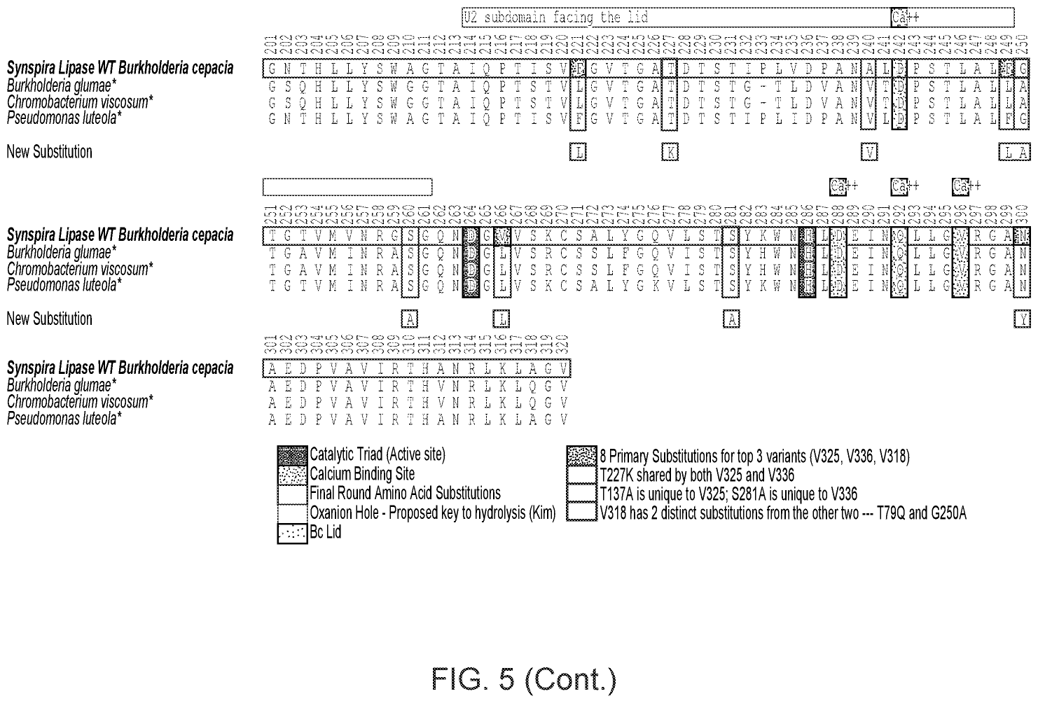

FIG. 4 depicts a sequence alignment showing the conservation of amino acids among the lipase sequences of Pseudomonas aeruginosa PAO1 (family I.1, SEQ ID NO: 29), Pseudomonas fluorescens (family I.1, SEQ ID NO: 30), Burkholderia cepacia (family I.2, SEQ ID NO: 1), Burkholderia glumae (family I.2, SEQ ID NO: 31), Chromobacterium viscosum (family I.2, SEQ ID NO:32), Pseudomonas luteola (family I.2, SEQ ID NO: 33), Pseudomonas fluorescens ABA72135 (family I.1, SEQ ID NO: 34), Pseudomonas fluorescens AEV60646 (family I.1, SEQ ID NO: 35), Pseudomonas sp WP-015093259 (family I.3, SEQ ID NO: 36), Pseudomonas fragi CAA32193 (family I.1, SEQ ID NO: 37), Pseudomonas fragi CAC07191 (family I.1, SEQ ID NO: 38), Pseudomonas stutzeri (SEQ ID NO: 41) and Pseudomonas mendocina LipA (SEQ ID NO: 42). The amino acid residues that constitute the catalytic triad (active site; dark shading) and calcium binding site (light shading), are depicted in the figure. Substitutions made in the lipase engineering (see, Example 7) are shown relative to the wild type B. cepacia sequence (boxes without shading). FIG. 5 depicts a sequence alignment showing the conservation of residues between Burkholderia cepacia (family I.2), Burkholderia glumae (family I.2, SEQ ID NO: 31), Chromobacterium viscosum (family I.2, SEQ ID NO:32), and Pseudomonas luteola (family I.2, SEQ ID NO: 40), where the conserved amino acids that constitute the oxyanion hole, the lid, the subdomain, the catalytic triad and calcium binding site are identified. The locations of amino acid substitutions made in the lipase engineering (see, Example 7) relative to the wild type B. cepacia sequence are depicted in the figure (boxes with dark outlines).

Family I.2 contains the lipase derived from Burkholderia cepacia (a/k/a Pseudomonas cepacia lipase), a gram-negative bacteria. The B. cepacia lipase provides a good starting point for enzyme engineering because it (1) has high activity against long-chain polyunsaturated fatty acids such as DHA, (ii) has a broad level of activity at physiologically relevant pH ranges in the gastrointestinal tract, (iii) is active with and without bile salts or minerals such as calcium, (iv) does not require co-lipase for catalytic activity, and (v) catalyzes the hydrolysis of triglycerides to produce two fatty acids and a 2-monoglycerides with a greater level of activity against the sn-1 and sn-3 regions of the triglyceride thereby mimicking human pancreatic lipase. B. cepacia lipase comprises about 320 amino acid residues and has an estimated molecular mass of about 33 kDa, and has structural features that are conserved among lipases. In particular, B. cepacia lipase contains an active site cleft containing the catalytic triad (the conserved serine, histidine and aspartic acid residues) and a lid that opens to expose the active site to permit entry of a triglyceride to be hydrolyzed or closes to close the active site. Other conserved features of the lipase include an oxyanion hole and a calcium ion binding site. The conservation of these structural features among family I.1 lipases, family I.2 lipases and family I.3 lipases suggest that these lipases share the same mechanisms of catalysis and interfacial activation (Kim et al. (1997) supra; Nardini et al. (2000) supra; Barbe et al. (2009) PROTEINS 77:509-523; Schrag et al. (1991), supra). Without wishing to be bound by theory, it is contemplated that the interfacial activation of the lipase results primarily from conformational changes in the lipase which expose the active site and provide a hydrophobic surface for interaction with the triglyceride substrate. Crystallographic and biochemical studies have shown that the mechanism of hydrolysis by lipases is similar to that of serine proteases. In both cases, it is believed that an oxyanion created during hydrolysis is located in the so-called ‘oxyanion hole’ when the lipase is in the open lid conformation (Kim et al. (1997), supra).

As discussed in more detail below, the B. cepacia lipase was subjected to rounds of mutagenesis as discussed in Examples 1, 6 and 7, which resulted in a number of amino acid substitutions that improved one or more properties of the B. cepacia lipase, which achieved certain design objectives, including creating a lipase that has one or more of (i) pH activity in the range of pH 3.0 to pH 7.0, (ii) high substate specificity and activity against certain long-chain polyunsaturated fatty acids (e.g., docosahexaenoic acid (DHA) and eicosapentaenoic acid (EPA) and long-chain triglycerides (e.g., oleic acid in olive oil), (iii) does not require co-factors (e.g., a co-lipase or pro-lipase), (iv) reduced or no dependence on bile-salts or minerals for activity, (v) high specific activity, (vi) thermostable in the temperature range of, for example, 35-40° C., and (vii) proteolytically stable.

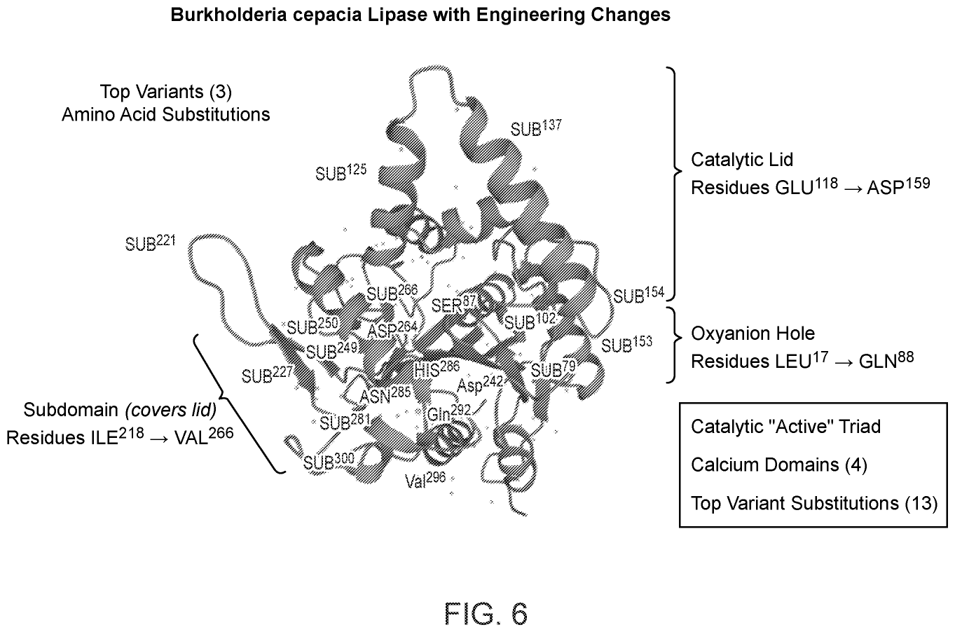

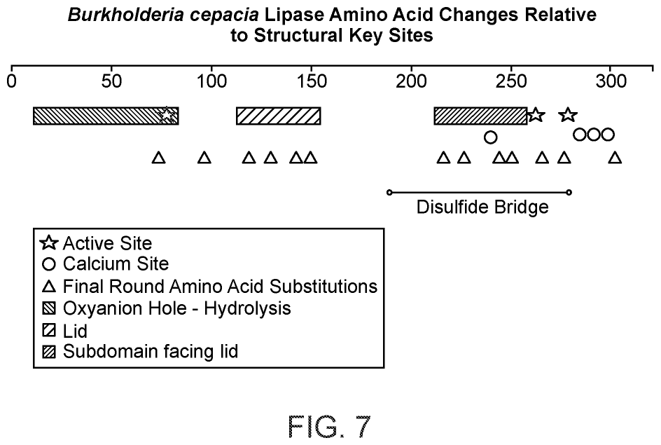

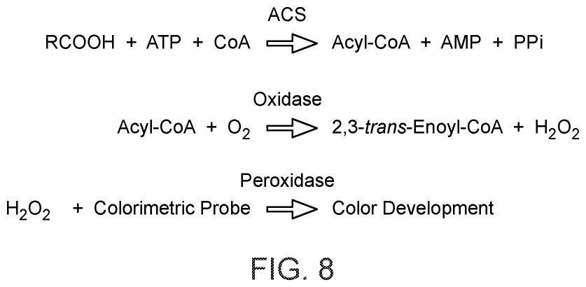

Initially, 57 amino acid substitutions were identified that enhanced the range of pH and proteolytic survivability (see, Example 1). Sixteen of the substitutions were maintained and additional substitutions were made resulting in certain combinations of substitutions that enhanced proteolytic and pH survivability (see, Example 6). Finally, various combinations of 18 of the substitutions initially identified were tested, which identified certain combinations of substitutions that enhanced pH stability (survivability against stomach acid at pH 3.0) and proteolytic stability (survivability against pepsin at pH 3.6 and Aspergillus melleus protease at pH 6.4) (see, Example 7). Based on these studies, certain amino acid substitutions and combinations of such substitutions that enhanced one or more properties of the lipase were found to be located on the lid, subdomain, and oxyanion hole of the lipase, which are depicted in the sequence alignment of FIG. 5, the three-dimensional ribbon model of the enzyme as shown in FIG. 6 or in the schematic representation the enzyme (FIG. 7).

### II. Recombinant Mutant Lipases

Among other things, the invention provides recombinant mutant lipases that are useful, for example, in treating disorders associated with a reduced ability to digest or absorb lipids, resulting in an elevated amount of undigested lipid in a subject, for example, disorders in which a subject exhibits low level secretion of pancreatic enzymes or has a physiological condition that affects fat hydrolysis or fat absorption (e.g., reduced gastric, duodenal, liver, bile, or gallbladder function; reduced gastrointestinal transit, motility, mixing, emptying; or reduced intestinal mucosa function (e.g., induced by mucosal damage). In certain embodiments, the lipase comprises (i) increased stability at acidic pH (e.g., pH 3.0 or 4.0) relative to a corresponding wild-type microbial lipase enzyme, (ii) increased stability in the presence of a protease (e.g., a serine protease and/or an aspartic protease) relative to the corresponding wild-type microbial lipase enzyme, (iii) activity for a sufficient length of time to transit to GI tract (e.g., a half life between about 75 and 225 minutes), or (iv) at least 60%, 70%, 80%, 85%, 90%, 95%, 96%, 97%, 98%, 99%, or 100% of the enzymatic activity of the corresponding wild-type microbial lipase enzyme.

In certain embodiments, the lipase is a α/β-hydrolase lipase and optionally or in addition may comprise a serine-histidine-aspartate active triad. In certain embodiments, the lipase comprises a hydrophobic lid that opens to allow for the binding and/or hydrolysis of a lipid. The hydrophobic lid may open sufficiently to allow for the binding and/or hydrolysis of a triglyceride having a chain length of more than eight carbons.

In certain embodiments, the lipase comprises a calcium binding site, wherein, when calcium is bound to the calcium binding site, the lipase is stabilized. In certain embodiments, the lipase comprises an oxyanion hole, wherein the oxyanion hole stabilizes a negatively charged intermediate generated during fatty acid bond hydrolysis. In certain embodiments, the lipase is a fungal lipase or a bacterial lipase. In certain embodiments, the lipase is a Family I bacterial lipase, and can be an I.1, I.2, or I.3 subfamily bacterial lipase, e.g., a I.1 or I.2 subfamily bacterial lipase, or a I.2 subfamily bacterial lipase.

In certain embodiments, the lipase is a Burkholderia, Pseudomonas, or Chromobacterium lipase. In certain embodiments, the lipase is a B. cepacia, Burkholderia glumae, Pseudomonas fluorescens, Pseudomonas aeruginosa, Pseudomonas luteola, or Chromobacterium viscosum lipase. In certain embodiments, the lipase is a B. cepacia lipase.

In certain embodiments, the lipase comprises a S residue at a position corresponding to position 87 of wild-type B. cepacia (S87), a D residue at a position corresponding to position 264 of wild-type B. cepacia (D264), and a H residue at a position corresponding to position 286 of wild-type B. cepacia (H286), which represents conserved amino acids between lipase subfamilies I.1 and I.2 (see, FIG. 4).

Unless stated otherwise, as used herein, wild-type B. cepacia lipase refers a B. cepacia lipase having the amino acid sequence of SEQ ID NO: 1, or a functional fragment thereof that digests a long-chain triglyceride substrate into fatty acids.

SEQ ID NO: 1 (wild-type B. cepacia lipase):
ADNYAATRYPIILVHGLIGTDKYAGVLEYWYGIQEDLQQRGATVY
VANLSGFQSDDGPNGRGEQLLAYVKTVLAATGATKVNLVGHSQGG
LTSRYVAAVAPDLVASVTTIGTPHRGSEFADFVQGVLAYDPTGLS
STVIAAFVNVFGILTSSSNNTNQDALAALKTITTAQAATYNQNYP
SAGIGAPGSCQTGAPTETVGGNTHLLYSWAGTAIQPTISVEGVTG
ATDTSTIPLVDPANALDPSTLALFGTGTVMVNRGSGQNDGVVSKC
SALYGQVLSTSYKWNHLDEINQLLGVRGANAEDPVAVIRTHANRL
KLAGV

As used herein, the term “functional fragment” is understood to be a protein fragment of a lipase that has at least 50%, 60%, 70%, 80%, 90%, 95%, or 98% of the activity of a corresponding full length lipase to digest a long-chain triglyceride substrate into fatty acids.

In certain embodiments, the lipase is not cross-linked and/or crystallized.

In one aspect, the invention provides a recombinant mutant lipase that comprises at least one (e.g., one, two, three, four, five, six, seven, eight, nine, ten, eleven, twelve, or thirteen, e.g., 2-13, 3-13, 4-13, 5-13, 6-13, 7-13, 8-13, 9-13, 10-13, 11-13, 12-13, 2-12, 3-12, 4-12, 5-12, 6-12, 7-12, 8-12, 9-12, 10-12, 11-12, 2-11, 3-11, 4-11, 5-11, 6-11, 7-11, 8-11, 9-11, 10-11, 2-10, 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-6, 3-6, 4-6, or 5-6) mutation(s) at a position corresponding to wild type B. cepacia lipase of SEQ ID NO: 1, wherein the at least one mutation is selected from a substitution of a residue at a position corresponding to position 39 of wild-type B. cepacia lipase; a substitution of a residue at a position corresponding to position 79 of wild-type B. cepacia lipase; a substitution of a residue at a position corresponding to position 102 of wild-type B. cepacia lipase; a substitution of a residue at a position corresponding to position 125 of wild-type B. cepacia lipase; a substitution of a residue at a position corresponding to position 128 of wild-type B. cepacia lipase; a substitution of a residue at a position corresponding to position 137 of wild-type B. cepacia lipase; a substitution of a residue at a position corresponding to position 138 of wild-type B. cepacia lipase; a substitution of a residue at a position corresponding to position 153 of wild-type B. cepacia lipase; a substitution of a residue at a position corresponding to position 154 of wild-type B. cepacia lipase; a substitution of a residue at a position corresponding to position 161 of wild-type B. cepacia lipase; a substitution of a residue at a position corresponding to position 170 of wild-type B. cepacia lipase; a substitution of a residue at a position corresponding to position 221 of wild-type B. cepacia lipase; a substitution of a residue at a position corresponding to position 227 of wild-type B. cepacia lipase; a substitution of a residue at a position corresponding to position 240 of wild-type B. cepacia lipase; a substitution of a residue at a position corresponding to position 249 of wild-type B. cepacia lipase; a substitution of a residue at a position corresponding to position 250 of wild-type B. cepacia lipase; a substitution of a residue at a position corresponding to position 260 of wild-type B. cepacia lipase; a substitution of a residue at a position corresponding to position 266 of wild-type B. cepacia lipase; a substitution of a residue at a position corresponding to position 281 of wild-type B. cepacia lipase; a substitution of a residue at a position corresponding to position 300 of wild-type B. cepacia lipase; or a combination of any of the foregoing substitutions.

In certain embodiments, the residue at a position corresponding to position 39 of wild-type B. cepacia lipase is substituted by R, H, or K; the residue at a position corresponding to position 79 of wild-type B. cepacia lipase is substituted by Q, N, or C; the residue at a position corresponding to position 102 of wild-type B. cepacia lipase is substituted by Q, N, or C; the residue at a position corresponding to position 125 of wild-type B. cepacia lipase is substituted by N, C, Q, S, or T; the residue at a position corresponding to position 128 of wild-type B. cepacia lipase is substituted by N, C, Q, S, or T; the residue at a position corresponding to position 137 of wild-type B. cepacia lipase is substituted by A, I, L, M, or V; the residue at a position corresponding to position 138 of wild-type B. cepacia lipase is substituted by A, I, L, M, or V; the residue at a position corresponding to position 153 of wild-type B. cepacia lipase is substituted by N, C, Q, S, or T; the residue at a position corresponding to position 154 of wild-type B. cepacia lipase is substituted by R, H, or K; the residue at a position corresponding to position 161 of wild-type B. cepacia lipase is substituted by A, I, L, M, or V; the residue at a position corresponding to position 170 of wild-type B. cepacia lipase is substituted by N, C, Q, S, or T; the residue at a position corresponding to position 221 of wild-type B. cepacia lipase is substituted by A, I, L, M, or V; the residue at a position corresponding to position 227 of wild-type B. cepacia lipase is substituted by R, H, or K; the residue at a position corresponding to position 240 of wild-type B. cepacia lipase is substituted by A, I, L, M, or V; the residue at a position corresponding to position 249 of wild-type B. cepacia lipase is substituted by A, I, L, M, or V; the residue at a position corresponding to position 250 of wild-type B. cepacia lipase is substituted by A, I, L, M, or V; the residue at a position corresponding to position 260 of wild-type B. cepacia lipase is substituted by A, I, L, M, or V; the residue at a position corresponding to position 266 of wild-type B. cepacia lipase is substituted by A, I, L, M, or V; the residue at a position corresponding to position 281 of wild-type B. cepacia lipase is substituted by A, I, L, M, or V; the residue at a position corresponding to position 300 of wild-type B. cepacia lipase is substituted by F, W, or Y; or the lipase comprises a combination of any of the foregoing substitutions.

In certain embodiments, the residue at a position corresponding to position 39 of wild-type B. cepacia lipase is substituted by R; the residue at a position corresponding to position 79 of wild-type B. cepacia lipase is substituted by Q; the residue at a position corresponding to position 102 of wild-type B. cepacia lipase is substituted by Q; the residue at a position corresponding to position 125 of wild-type B. cepacia lipase is substituted by S; the residue at a position corresponding to position 128 of wild-type B. cepacia lipase is substituted by N; the residue at a position corresponding to position 137 of wild-type B. cepacia lipase is substituted by A; the residue at a position corresponding to position 138 of wild-type B. cepacia lipase is substituted by I; the residue at a position corresponding to position 153 of wild-type B. cepacia lipase is substituted by N; the residue at a position corresponding to position 154 of wild-type B. cepacia lipase is substituted by H; the residue at a position corresponding to position 161 of wild-type B. cepacia lipase is substituted by A; the residue at a position corresponding to position 170 of wild-type B. cepacia lipase is substituted by S; the residue at a position corresponding to position 221 of wild-type B. cepacia lipase is substituted by L; the residue at a position corresponding to position 227 of wild-type B. cepacia lipase is substituted by K; the residue at a position corresponding to position 240 of wild-type B. cepacia lipase is substituted by V; the residue at a position corresponding to position 249 of wild-type B. cepacia lipase is substituted by L; the residue at a position corresponding to position 250 of wild-type B. cepacia lipase is substituted by A; the residue at a position corresponding to position 260 of wild-type B. cepacia lipase is substituted by A; the residue at a position corresponding to position 266 of wild-type B. cepacia lipase is substituted by L; the residue at a position corresponding to position 281 of wild-type B. cepacia lipase is substituted by A; the residue at a position corresponding to position 300 of wild-type B. cepacia lipase is substituted by Y; or the lipase comprises a combination of any of the foregoing substitutions.

In certain embodiments, the lipase comprises a substitution of a Q residue at a position corresponding to position 39 of wild-type B. cepacia lipase (Q39); a substitution of a T residue at a position corresponding to position 79 of wild-type B. cepacia lipase (T79); a substitution of a D residue at a position corresponding to position 102 of wild-type B. cepacia lipase (D102); a substitution of a G residue at a position corresponding to position 125 of wild-type B. cepacia lipase (G125); a substitution of an A residue at a position corresponding to position 128 of wild-type B. cepacia lipase (A128); a substitution of a T residue at a position corresponding to position 137 of wild-type B. cepacia lipase (T137); a substitution of a V residue at a position corresponding to position 138 of wild-type B. cepacia lipase (V138); a substitution of an S residue at a position corresponding to position 153 of wild-type B. cepacia lipase (S153); a substitution of a N residue at a position corresponding to position 154 of wild-type B. cepacia lipase (N154); a substitution of an L residue at a position corresponding to position 161 of wild-type B. cepacia lipase (L161); a substitution of an A residue at a position corresponding to position 170 of wild-type B. cepacia lipase (A170); a substitution of a F residue at a position corresponding to position 221 of wild-type B. cepacia lipase (F221); a substitution of a T residue at a position corresponding to position 227 of wild-type B. cepacia lipase (T227); a substitution of an A residue at a position corresponding to position 240 of wild-type B. cepacia lipase (A240); a substitution of a F residue at a position corresponding to position 249 of wild-type B. cepacia lipase (F249); a substitution of a G residue at a position corresponding to position 250 of wild-type B. cepacia lipase (G250); a substitution of an S residue at a position corresponding to position 260 of wild-type B. cepacia lipase (S260); a substitution of a V residue at a position corresponding to position 266 of wild-type B. cepacia lipase (V266); a substitution of an S residue at a position corresponding to position 281 of wild-type B. cepacia lipase (S281); a substitution of an N residue at a position corresponding to position 300 of wild-type B. cepacia lipase (N300); or a combination of any of the foregoing substitutions.

In certain embodiments, the Q residue at a position corresponding to position 39 of wild-type B. cepacia lipase is substituted by R (Q39R); the T residue at a position corresponding to position 79 of wild-type B. cepacia lipase is substituted by Q (T79Q); the D residue at a position corresponding to position 102 of wild-type B. cepacia lipase is substituted by Q (D102Q); the G residue at a position corresponding to position 125 of wild-type B. cepacia lipase is substituted by S (G125S); the A residue at a position corresponding to position 128 of wild-type B. cepacia lipase is substituted by N (A128N); the T residue at a position corresponding to position 137 of wild-type B. cepacia lipase is substituted by A (T137A); the V residue at a position corresponding to position 138 of wild-type B. cepacia lipase is substituted by I (V1381); the S residue at a position corresponding to position 153 of wild-type B. cepacia lipase is substituted by N (S153N); the N residue at a position corresponding to position 154 of wild-type B. cepacia lipase is substituted by H (N154H); the L residue at a position corresponding to position 161 of wild-type B. cepacia lipase is substituted by A (L161A); the A residue at a position corresponding to position 170 of wild-type B. cepacia lipase is substituted by S (A170S); the F residue at a position corresponding to position 221 of wild-type B. cepacia lipase is substituted by L (F221L); the T residue at a position corresponding to position 227 of wild-type B. cepacia lipase is substituted by K (T227K); the A residue at a position corresponding to position 240 of wild-type B. cepacia lipase is substituted by V (A240V); the F residue at a position corresponding to position 249 of wild-type B. cepacia lipase is substituted by L (F249L); the G residue at a position corresponding to position 250 of wild-type B. cepacia lipase is substituted by A (G250A); the S residue at a position corresponding to position 260 of wild-type B. cepacia lipase is substituted by A (S260A); the V residue at a position corresponding to position 266 of wild-type B. cepacia lipase is substituted by L (V266L); the S residue at a position corresponding to position 281 of wild-type B. cepacia lipase is substituted by A (S281A); the N residue at a position corresponding to position 300 of wild-type B. cepacia lipase is substituted by Y (N300Y); or the lipase comprises a combination of any of the foregoing substitutions.

In certain embodiments, one or more mutations may be conservative substitutions relative to wild type B. cepacia lipase of SEQ ID NO: 1, whereas in certain other embodiments, one or more mutations may be non-conservative substitutions relative to wild type B. cepacia lipase of SEQ ID NO: 1. As used herein, the term “conservative substitution” refers to a substitution with a structurally similar amino acid.

In certain embodiments, the substitution of a given amino acid is with a hydrophobic amino acid (e.g., A, I, L, M, or V), a positively charged amino acid (e.g., K, R or H), a negatively charged amino acid (e.g., D or E), a polar neutral amino acid (e.g., N, C, Q, S or T), an aromatic amino acid (e.g., F, Y or W) or a bulkier amino acid based on side chain volume or a smaller amino acid based on side chain volume. The amino acids are denoted in the single letter code.

Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix). Non conservative substitutions are amino acid substitutions that are not conservative substitutions.

In one aspect, the recombinant mutant lipase enzyme comprises one or substitutions from TABLE 1, wherein the positions of the substitutions are shown relative to wild type B. cepacia (e.g., SEQ ID NO: 1).

TABLE 1

| Position relative | Exemplary | Exemplary |
|---|---|---|
| to wild type | Substitutions - | Substitutions - |
| B. cepacia (SEQ ID NO: 1) | Embodiment 1 | Embodiment 2 |
| I12 | A, I, L, M, or V | V |
| A24 | G or P | G |
| V26 | A, I, L, M, or V | I |
| Q34 | N, C, Q, S, or T | T |
| E35 | N, C, Q, S, or T | Q or S |
| Q39 | A, I, L, M, or V | A or R |
| | or R, H, or K | |
| R40 | N, C, Q, S, or T | N |
| T43 | R, H, or K | K |
| A75 | N, C, Q, S, or T | T |
| T79 | A, I, L, M, or V | A, Q, or S |
| | or N, C, Q, S, or T | |
| V84 | A, I, L, M, or V | I |
| L91 | A, I, L, M, or V | M |
| T92 | N, C, Q, S, or T | S or A |
| | or A, I, L, M, or V | |
| D102 | N, C, Q, S, or T | N or Q |
| G125 | D or E | D, N, or S |
| | or N, C, Q, S, or T | |
| V126 | A, I, L, M, or V | A |
| A128 | N, C, Q, S, or T | N |
| Y129 | N, C, Q, S, or T | N |
| L134 | A, I, L, M, or V | A |
| S136 | A, I, L, M, or V | L |
| T137 | A, I, L, M, or V | A or S |
| | or N, C, Q, S, or T | |
| V138 | A, I, L, M, or V | I |
| I139 | A, I, L, M, or V | L |
| V143 | A, I, L, M, or V | A |
| N144 | D or E | D |
| F146 | A, I, L, M, or V | A |
| I148 | A, I, L, M, or V | L |
| N154 | R, H, or K | H |
| N155 | D or E | D |
| N157 | A, I, L, M, or V | I |
| D159 | N, C, Q, S, or T | N |
| L161 | A, I, L, M, or V | A |
| K165 | N, C, Q, S, or T | Q |
| A170 | N, C, Q, S, or T | S |
| Q171 | R, H, or K | R |
| T174 | R, H, or K | R |
| Q177 | A, I, L, M, or V | A or K |
| | or R, H, or K | |
| N178 | R, H, or K | R |
| T196 | A, I, L, M, or V | A |
| T198 | F, W, or Y | F |
| G200 | N, C, Q, S, or T | N |
| T203 | R, H, or K | K or R |
| A210 | G or P | G |
| V220 | A, I, L, M, or V | A |
| F221 | A, I, L, M, or V | L |
| T224 | N, C, Q, S, or T | Q or S |
| T227 | R, H, or K | K or N |
| | or N, C, Q, S, or T | |
| G225 | A, I, L, M, or V | L |
| I232 | A, I, L, M, or V | L |
| L234 | A, I, L, M, or V | A, P, or V |
| | or G or P | |
| V235 | A, I, L, M, or V | I |
| P237 | A, I, L, M, or V | V |
| A240 | A, I, L, M, or V | V |
| F249 | A, I, L, M, or V | L |
| G250 | A, I, L, M, or V | A |
| G252 | A, I, L, M, or V | A |
| T253 | A, I, L, M, or V | A |
| S260 | A, I, L, M, or V | A |
| Q262 | G or P | P |
| V266 | A, I, L, M, or V | L |
| Q276 | R, H, or K | K |
| S279 | G or P | G |
| S281 | A, I, L, M, or V | A or N |
| | or N, C, Q, S, or T | |
| L287 | A, I, L, M, or V | I or V |
| N300 | F, W, or Y | Y |
| V305 | A, I, L, M, or V | I |
| A306 | N, C, Q, S, or T | T |

In another aspect, the invention provides a recombinant mutant lipase comprising at least one (e.g., at least one, at least two, at least three, at least four, at least five, at least six, at least seven, at least eight, at least nine, at least ten, or at least 11 different mutations) mutation(s). In certain embodiments, the invention provides a recombinant mutant lipase comprising at least one (e.g., at least one, at least two, at least three, at least four, at least five, at least six, at least seven, at least eight, at least nine, at least ten, or at least eleven different mutations) mutation(s) selected from TABLE 1. In certain embodiments, one or more mutations may be conservative substitutions relative to wild type B. cepacia lipase of SEQ ID NO: 1, whereas in certain other embodiments, one or more mutations may be non-conservative substitutions relative to wild type B. cepacia lipase of SEQ ID NO: 1.

In another aspect, the recombinant mutant lipase comprises up to 11 substitutions listed in a given row of TABLE 2, wherein the positions of the substitutions are depicted relative to wild type B. cepacia (e.g., SEQ ID NO: 1).

TABLE 2

| | | | | | | | | | | | |
|---|---|---|---|---|---|---|---|---|---|---|---|
| 1 | T79Q | D102Q | G125S | T137A | S153N | N154H | F221L | V266L | N300Y | | |
| 2 | T79Q | D102Q | G125S | T137A | N154H | F221L | F249L | V266L | N300Y | | |
| 3 | T79Q | D102Q | G125S | S153N | N154H | F221L | F249L | V266L | N300Y | | |
| 4 | D102Q | G125S | T137A | S153N | N154H | F221L | F249L | V266L | N300Y | | |
| 5 | T79Q | D102Q | G125S | T137A | S153N | N154H | F221L | V266L | N300Y | T227K | |
| 6 | T79Q | D102Q | G125S | T137A | S153N | N154H | F221L | F249L | V266L | N300Y | |
| 7 | T79Q | D102Q | G125S | T137A | S153N | N154H | F221L | V266L | N300Y | G250A | |
| 8 | T79Q | D102Q | G125S | T137A | S153N | N154H | F221L | V266L | S281A | N300Y | |
| 9 | T79Q | D102Q | G125S | T137A | N154H | F221L | F249L | V266L | N300Y | T227K | |
| 10 | T79Q | D102Q | G125S | T137A | N154H | F221L | V266L | N300Y | T227K | G250A | |
| 11 | T79Q | D102Q | G125S | T137A | N154H | F221L | V266L | S281A | N300Y | T227K | |
| 12 | T79Q | D102Q | G125S | T137A | N154H | F221L | F249L | V266L | N300Y | G250A | |
| 13 | T79Q | D102Q | G125S | T137A | N154H | F221L | F249L | V266L | S281A | N300Y | |
| 14 | T79Q | D102Q | G125S | T137A | N154H | F221L | V266L | S281A | N300Y | G250A | |
| 15 | T79Q | D102Q | G125S | S153N | N154H | F221L | F249L | V266L | N300Y | T227K | |
| 16 | T79Q | D102Q | G125S | S153N | N154H | F221L | V266L | N300Y | T227K | G250A | |
| 17 | T79Q | D102Q | G125S | S153N | N154H | F221L | V266L | S281A | N300Y | T227K | |
| 18 | T79Q | D102Q | G125S | S153N | N154H | F221L | F249L | V266L | N300Y | G250A | |
| 19 | T79Q | D102Q | G125S | S153N | N154H | F221L | F249L | V266L | S281A | N300Y | |
| 20 | T79Q | D102Q | G125S | S153N | N154H | F221L | V266L | S281A | N300Y | G250A | |
| 21 | T79Q | D102Q | G125S | N154H | F221L | F249L | V266L | N300Y | T227K | G250A | |
| 22 | T79Q | D102Q | G125S | N154H | F221L | F249L | V266L | S281A | N300Y | T227K | |
| 23 | T79Q | D102Q | G125S | N154H | F221L | V266L | S281A | N300Y | T227K | G250A | |
| 24 | T79Q | D102Q | G125S | N154H | F221L | F249L | V266L | S281A | N300Y | G250A | |
| 25 | D102Q | G125S | T137A | S153N | N154H | F221L | F249L | V266L | N300Y | T227K | |
| 26 | D102Q | G125S | T137A | S153N | N154H | F221L | V266L | N300Y | T227K | G250A | |
| 27 | D102Q | G125S | T137A | S153N | N154H | F221L | V266L | S281A | N300Y | T227K | |
| 28 | D102Q | G125S | T137A | S153N | N154H | F221L | F249L | V266L | N300Y | G250A | |
| 29 | D102Q | G125S | T137A | S153N | N154H | F221L | F249L | V266L | S281A | N300Y | |
| 30 | D102Q | G125S | T137A | S153N | N154H | F221L | V266L | S281A | N300Y | G250A | |
| 31 | D102Q | G125S | T137A | N154H | F221L | F249L | V266L | N300Y | T227K | G250A | |
| 32 | D102Q | G125S | T137A | N154H | F221L | F249L | V266L | S281A | N300Y | T227K | |
| 33 | D102Q | G125S | T137A | N154H | F221L | V266L | S281A | N300Y | T227K | G250A | |
| 34 | D102Q | G125S | T137A | N154H | F221L | F249L | V266L | S281A | N300Y | G250A | |
| 35 | D102Q | G125S | S153N | N154H | F221L | F249L | V266L | N300Y | T227K | G250A | |
| 36 | D102Q | G125S | S153N | N154H | F221L | F249L | V266L | S281A | N300Y | T227K | |
| 37 | D102Q | G125S | S153N | N154H | F221L | V266L | S281A | N300Y | T227K | G250A | |
| 38 | D102Q | G125S | S153N | N154H | F221L | F249L | V266L | S281A | N300Y | G250A | |
| 39 | D102Q | G125S | N154H | F221L | F249L | V266L | S281A | N300Y | T227K | G250A | |
| 40 | D102Q | G125S | T137A | S153N | N154H | F221L | F249L | V266L | N300Y | A170S | |
| 41 | D102Q | G125S | T137A | V138I | S153N | N154H | F221L | F249L | V266L | N300Y | |
| 42 | D102Q | G125S | T137A | S153N | N154H | F221L | F249L | V266L | N300Y | Q39R | |
| 43 | G125S | T137A | S153N | N154H | F221L | F249L | V266L | N300Y | | | |
| 44 | D102Q | G125S | T137A | S153N | N154H | F221L | F249L | V266L | N300Y | S260A | |
| 45 | D102Q | G125S | T137A | S153N | N154H | F221L | F249L | V266L | N300Y | A240V | |
| 46 | T79Q | D102Q | G125S | T137A | S153N | N154H | F221L | F249L | V266L | N300Y | G250A |

In certain embodiments, in any of the foregoing recombinant mutant lipases, the lipase comprises the following substitutions (i) D102Q, N154H, and F221L; (11) T79Q, V266L, and L287V; (iii) L91M, V220A, and V266L; (iv) G125D), D159N, and F249L; (v) Q39A, T137A, and F249L; (vi) D102Q, G125S, N154H, F221L, V266L, and N300Y; (vii) D102Q, T137A, F221L, E35S, G250A, and V3051; (viii) D102Q, N154H, L161A, F221L, S281A, and 1218A; (ix) L91M, D102Q, A128N, N154H, F221L, and Q177A; or (x) D102Q, S153N, N154H, F221L, Q39R, and T92S.

In certain embodiments, in any of the foregoing recombinant mutant lipases, the lipase comprises the following substitutions (i) D102Q, N154H, and F221L; (ii) D102Q, G125S, N154H, F221L, V266L, and N300Y; (iii) T79Q, D102Q, G125S, T137A, N154H, F221L, T227K, F249L, V266L, and N300Y; (iv) T79Q, D102Q, G125S, T137A, N154H, F221L, T227K, V266L, S281A, and N300Y; (v) T79Q, D102Q, G125S, S153N, N154H, F221L, T227K, V266L, S281A, and N300Y; (vi) T79Q, D102Q, G125S, S153N, N154H, F221L, F249L, G250A, V266L, and N300Y; (vii) T79Q, D102Q, G125S, S153N, N154H, F221L, F249L, V266L, S281A, and N300Y; (viii) T79Q, D102Q, G125S, N154H, F221L, T227K, F249L, V266L, S281A, and N300Y; (ix) D102Q, G125S, T137A, S153N, N154H, F221L, T227K, F249L, V266L, and N300Y; (x) D102Q, G125S, T137A, S153N, N154H, F221L, T227K, G250A, V266L, and N300Y; (xi) D102Q, G125S, T137A, N154H, F221L, T227K, G250A, V266L, S281A, and N300Y; (xii) D102Q, G125S, S153N, N154H, F221L, T227K, F249L, G250A, V266L, and N300Y; or (xiii) D102Q, G125S, S153N, N154H, F221L, T227K, F249L, V266L, S281A, and N300Y, either alone or in combination with other substitutions.

The invention further provides a recombinant mutant lipase that comprises the following substitutions: D102Q, N154H, and F221L, e.g., a recombinant mutant B. cepacia lipase comprising the following amino acid sequence, e.g., a recombinant mutant lipase referred to as V130 herein:

(SEQ ID NO: 2)
ADNYAATRYPIILVHGLTGTDKYAGVLEYWYGIQEDLQQRGATVY
VANLSGFQSDDGPNGRGEQLLAYVKTVLAATGATKVNLVGHSQGG
LTSRYVAAVAPQLVASVTTIGTPHRGSEFADFVQGVLAYDPTGLS
STVIAAFVNVFGILTSSSHNTNQDALAALKTLTTAQAATYNQNYP
SAGLGAPGSCQTGAPTETVGGNTHLLYSWAGTAIQPTISVLGVTG
ATDTSTIPLVDPANALDPSTLALFGTGTVMVNRGSGQNDGVVSKC
SALYGQVLSTSYKWNHLDEINQLLGVRGANAEDPVAVIRTHANRL
KLAGV

The invention further provides a recombinant mutant lipase that comprises the following substitutions: D102Q, G125S, N154H, F221L, V266L, and N300Y, e.g., a recombinant mutant B. cepacia lipase comprising the following amino acid sequence, e.g., a recombinant mutant lipase referred to as V290 herein:

(SEQ ID NO: 3)
ADNYAATRYPIILVHGLIGTDKYAGVLEYWYGIQEDLQQRGATVY
VANLSGFQSDDGPNGRGEQLLAYVKTVLAATGATKVNLVGHSQGG
LTSRYVAAVAPQLVASVTTIGTPHRGSEFADFVQSVLAYDPTGLS
STVIAAFVNVEGILTSSSHNTNQDALAALKTLTTAQAATYNQNYP
SAGLGAPGSCQTGAPTETVGGNTHLLYSWAGTAIQPTISVLGVTG
ATDTSTIPLVDPANALDPSTLALFGTGTVMVNRGSGQNDGLVSKC
SALYGQVISTSYKWNHLDEINQLLGVRGAYAEDPVAVIRTHANRL
KLAGV.

The invention further provides a recombinant mutant lipase that comprises the following substitutions: T79Q, D102Q, G125S, T137A, N154H, F221L, T227K, F249L, V266L, N300Y, e.g., a recombinant mutant B. cepacia lipase comprising the following amino acid sequence, e.g., a recombinant mutant lipase referred to as V309 herein:

(SEQ ID NO: 4)
ADNYAATRYPIILVHGLIGTDKYAGVLEYWYGIQEDLQQRGATVY
VANLSGFQSDDGPNGRGEQLLAYVKTVLAATGAQKVNLVGHSQGG
LTSRYVAAVAPQLVASVTTIGTPHRGSEFADEVQSVLAYDPTGLS
SAVIAAFVNVFGILTSSSHNTNQDALAALKTLTTAQAATYNQNYP
SAGLGAPGSCQTGAPTETVGGNTHLLYSWAGTAIQPTISVLGVTG
AKDTSTIPLVDPANALDPSTLALLGTGTVMVNRGSGQNDGLVSKC
SALYGQVISTSYKWNHLDEINQLLGVRGAYAEDPVAVIRTHANRL
KLAGV.

The invention further provides a recombinant mutant lipase that comprises the following substitutions: T79Q, D102Q, G125S, T137A, N154H, F221L, T227K, V266L, S281A, and N300Y, e.g., a recombinant mutant B. cepacia lipase comprising the following amino acid sequence, e.g., a recombinant mutant lipase referred to as V311 herein:

(SEQ ID NO: 5)
ADNYAATRYPIILVHGLTGTDKYAGVLEYWYGIQEDLQQRGATVY
VANISGFQSDDGPNGRGEQLLAYVKTVLAATGAQKVNLVGHSQGG
LTSRYVAAVAPQLVASVITIGTPHRGSEFADEVQSVLAYDPTGLS
SAVIAAFVNVFGILTSSSHNTNQDALAALKTITTAQAATYNQNYP
SAGIGAPGSCQTGAPTETVGGNTHLLYSWAGTAIQPTISVLGVTG
AKDTSTIPLVDPANALDPSTLALFGTGTVMVNRGSGQNDGLVSKC
SALYGQVLSTAYKWNHLDEINQLLGVRGAYAEDPVAVIRTHANRL
KLAGV

The invention further provides a recombinant mutant lipase that comprises the following substitutions: T79Q, D102Q, G125S, S153N, N154H, F221L, T227K, V266L, S281A, and N300Y, e.g., a recombinant mutant B. cepacia lipase comprising the following amino acid sequence, e.g., a recombinant mutant lipase referred to as V317 herein:

(SEQ ID NO: 6)
ADNYAATRYPIILVHGLIGTDKYAGVLEYWYGIQEDLQQRGATVY
VANLSGFQSDDGPNGRGEQLLAYVKTVLAATGAQKVNLVGHSQGG
LTSRYVAAVAPQLVASVTTIGTPHRGSEFADEVQSVLAYDPTGLS
STVIAAFVNVFGILTSSNHNTNQDALAALKTLTTAQAATYNQNYP
SAGLGAPGSCQTGAPTETVGGNTHLLYSWAGTAIQPTISVLGVTG
AKDTSTIPLVDPANALDPSTLALFGTGTVMVNRGSGQNDGLVSKC
SALYGQVLSTAYKWNHLDEINQLLGVRGAYAEDPVAVIRTHANRL
KLAGV

The invention further provides a recombinant mutant lipase that comprises the following substitutions: T79Q, D102Q, G125S, S153N, N154H, F221L, F249L, V266L, N300Y, and G250A, e.g., a recombinant mutant B. cepacia lipase comprising the following amino acid sequence, e.g., a recombinant mutant lipase referred to as V318 herein:

(SEQ ID NO: 7)
ADNYAATRYPIILVHGLTGTDKYAGVLEYWYGIQEDLQQRGATVY
VANISGFQSDDGPNGRGEQLLAYVKTVLAATGAQKVNLVGHSQGG
LTSRYVAAVAPQLVASVTTIGTPHRGSEFADEVQSVLAYDPTGLS
STVIAAFVNVFGILTSSNHNTNQDALAALKTITTAQAATYNQNYP
SAGLIGAPGSCQTGAPTETVGGNTHLLYSWAGTAIQPTISVLGVT
GATDTSTIPLVDPANALDPSTLALLATGTVMVNRGSGQNDGLVSK
CSALYGQVISTSYKWNHLDEINQLLGVRGAYAEDPVAVIRTHANR
LKLAGV

The invention further provides a recombinant mutant lipase that comprises the following substitutions: T79Q, D102Q, G125S, S153N, N154H, F221L, F249L, V266L, S281A, and N300Y, e.g., a recombinant mutant B. cepacia lipase comprising the following amino acid sequence, e.g., a recombinant mutant lipase referred to as V319 herein:

(SEQ ID NO: 8)
ADNYAATRYPIILVHGLTGTDKYAGVLEYWYGIQEDLQQRGATVY
VANLSGFQSDDGPNGRGEQLLAYVKTVLAATGAQKVNLVGHSQGG
LTSRYVAAVAPQLVASVTTIGTPHRGSEFADFVQSVLAYDPTGLS
STVIAAFVNVFGILTSSNHNTNQDALAALKTLTTAQAATYNQNYP
SAGLGAPGSCQTGAPTETVGGNTHLLYSWAGTAIQPTISVLGVTG
ATDTSTIPLVDPANALDPSTLALLGTGTVMVNRGSGQNDGLVSKC
SALYGQVLSTAYKWNHLDEINQLLGVRGAYAEDPVAVIRTHANRL
KLAGV

The invention further provides a recombinant mutant lipase that comprises the following substitutions: T79Q, D102Q, G125S, N154H, F221L, T227K, F249L, V266L, S281A, and N300Y, e.g., a recombinant mutant B. cepacia lipase comprising the following amino acid sequence, e.g., a recombinant mutant lipase referred to as V322 herein:

(SEQ ID NO: 9)
ADNYAATRYPIILVHGLIGTDKYAGVLEYWYGIQEDLQQRGATVY
VANLSGFQSDDGPNGRGEQLLAYVKTVLAATGAQKVNLVGHSQGG
LTSRYVAAVAPQLVASVTTIGTPHRGSEFADFVQSVLAYDPTGLS
STVIAAFVNVEGILTSSSHNTNQDALAALKTLTTAQAATYNQNYP
SAGLGAPGSCQTGAPTETVGGNTHLLYSWAGTAIQPTISVLGVTG
AKDTSTIPLVDPANALDPSTLALLGTGTVMVNRGSGQNDGLVSKC
SALYGQVLSTAYKWNHLDEINQLLGVRGAYAEDPVAVIRTHANRL
KLAGV

The invention further provides a recombinant mutant lipase that comprises the following substitutions: D102Q, G125S, T137A, S153N, N154H, F221L, T227K, F249L, V266L, and N300Y, e.g., a recombinant mutant B. cepacia lipase comprising the following amino acid sequence, e.g., a recombinant mutant lipase referred to as V325 herein:

(SEQ ID NO: 10)
ADNYAATRYPIILVHGLIGTDKYAGVLEYWYGIQEDLQQRGATVY
VANLSGFQSDDGPNGRGEQLLAYVKTVLAATGATKVNLVGHSQGG
LTSRYVAAVAPQLVASVTTIGTPHRGSEFADEVQSVLAYDPTGLS
SAVIAAFVNVFGILTSSNHNTNQDALAALKTLTTAQAATYNQNYP
SAGLGAPGSCQTGAPTETVGGNTHLLYSWAGTAIQPTISVLGVTG
AKDTSTIPLVDPANALDPSTLALLGTGTVMVNRGSGQNDGLVSKC
SALYGQVISTSYKWNHLDEINQLLGVRGAYAEDPVAVIRTHANRL
KLAGV

The invention further provides a recombinant mutant lipase that comprises the following substitutions: D102Q, G125S, T137A, S153N, N154H, F221L, T227K, V266L, and N300Y, e.g., a recombinant mutant B. cepacia lipase comprising the following amino acid sequence, e.g., a recombinant mutant lipase referred to as V326 herein:

(SEQ ID NO: 11)
ADNYAATRYPIILVHGLIGTDKYAGVLEYWYGI QEDLQQRGATV
YVANLSGFQSDDGPNGRGEQLLAYVKTVLAATGATKVNLVGHSQG
GLTSRYVAAVAPQLVASVTTIGTPHRGSEFADEVQSVLAYDPTGL
SSAVIAAFVNVFGILTSSNHNTNQDALAALKTLTTAQAATYNQNY
PSAGLGAPGSCQTGAPTETVGGNTHLLYSWAGTAIQPTISVLGVT
GAKDTSTIPLVDPANALDPSTLALFATGTVMVNRGSGQNDGLVS
KCSALYGQVISTSYKWNHLDEINQLLGVRGAYAEDPVAVIRTHAN
RLKLAGV

The invention further provides a recombinant mutant lipase that comprises the following substitutions: D102Q, G125S, T137A, N154H, F221L, T227K, G250A, V266L, S281A, and N300Y, e.g., a recombinant mutant B. cepacia lipase comprising the following amino acid sequence, e.g., a recombinant mutant lipase referred to as V333 herein:

(SEQ ID NO: 12)
ADNYAATRYPIILVHGLIGTDKYAGVLEYWYGIQEDLQQRGATVY
VANISGFQSDDGPNGRGEQLLAYVKTVLAATGATKVNLVGHSQGG
LTSRYVAAVAPQLVASVTTIGTPHRGSEFADEVQSVLAYDPTGLS
SAVIAAFVNVFGILTSSSHNTNQDALAALKTITTAQAATYNQNYP
SAGLGAPGSCQTGAPTETVGGNTHLLYSWAGTAIQPTISVLGVTG
AKDTSTIPLVDPANALDPSTLALFATGTVMVNRGSGQNDGLVSKC
SALYGQVLSTAYKWNHLDEINQLLGVRGAYAEDPVAVIRTHANRL
KLAGV

The invention further provides a recombinant mutant lipase that comprises the following substitutions: D102Q, G125S, S153N, N154H, F221L, T227K, F249L, G250A V266L, and N300Y, e.g., a recombinant mutant B. cepacia lipase comprising the following amino acid sequence, e.g., a recombinant mutant lipase referred to as V335 herein:

(SEQ ID NO: 13)
ADNYAATRYPIILVHGLTGTDKYAGVLEYWYGIQEDLQQRGATVY
VANLSGFQSDDGPNGRGEQLLAYVKTVLAATGATKVNLVGHSQGG
LTSRYVAAVAPQLVASVTTIGTPHRGSEFADFVQSVLAYDPTGLS
STVIAAFVNVFGILTSSNHNTNQDALAALKTLTTAQAATYNQNYP
SAGLGAPGSCQTGAPTETVGGNTHLLYSWAGTAIQPTISVLGVTG
AKDTSTIPLVDPANALDPSTLALLATGTVMVNRGSGQNDGLVSKC
SALYGQVLSTSYKWNHLDEINQLLGVRGAYAEDPVAVIRTHANRL
KLAGV

The invention further provides a recombinant mutant lipase that comprises the following substitutions: D102Q, G125S, S153N, N154H, F221L, T227K, F249L, V266L, S281A, and N300Y, e.g., a recombinant mutant B. cepacia lipase comprising the following amino acid sequence, e.g., a recombinant mutant lipase referred to as V336 herein:

(SEQ ID NO: 14)
ADNYAATRYPIILVHGLIGTDKYAGVLEYWYGIQEDLQQRGATVY
VANLSGFQSDDGPNGRGEQLLAYVKTVLAATGATKVNLVGHSQGG
LTSRYVAAVAPQLVASVTTIGTPHRGSEFADFVQSVLAYDPTGL
SSTVIAAFVNVFGILTSSNHNTNQDALAALKTLTTAQAATYNQNY
PSAGLGAPGSCQTGAPTETVGGNTHLLYSWAGTAIQPTISVLGVT
GAKDTSTIPLVDPANALDPSTLALLGTGTVMVNRGSGQNDGLVSK
CSALYGQVLSTAYKWNHLDEINQLLGVRGAYAEDPVAVIRTHANR
LKLAGV

In certain embodiments, the lipase comprises the amino acid sequence of any one of SEQ ID NOs: 2-14, or an amino acid sequence that has at least 85%, 90%, 95%, 96%, 97%, 98%, or 99% sequence identity to any one of SEQ ID NOs: 2-14.

Sequence identity may be determined in various ways that are within the skill in the art, e.g., using publicly available computer software such as BLAST, BLAST-2, ALIGN or Megalign (DNASTAR) software. BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases, see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: −G, Cost to open gap [Integer]: default=5 for nucleotides/11 for proteins; −E, Cost to extend gap [Integer]: default=2 for nucleotides/1 for proteins; −q, Penalty for nucleotide mismatch [Integer]: default=−3; −r, reward for nucleotide match [Integer]: default=1; −e, expect value [Real]: default=10; —W, wordsize [Integer]: default=11 for nucleotides/28 for megablast/3 for proteins; −y, Dropoff (X) for blast extensions in bits: default=20 for blastn/7 for others; −X, X dropoff value for gapped alignment (in bits): default=15 for all programs, not applicable to blastn; and —Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

a. Recombinant Mutant Lipases with Increased Stability at Low pH

In certain embodiments, a recombinant mutant lipase has increased stability at acidic pH (e.g., pH 3.0 or 4.0) relative to a corresponding wild-type lipase enzyme. An increased stability at acidic pH allows a recombinant mutant lipase to survive the acidic conditions of the digestive system, especially the stomach. Normal pre-prandial stomach pH varies from about 1.5 to about 3.5, and postprandial pH increases to about 5. During the fed-state interval, there is a slow, but continuous emptying of the stomach contents though the pyloric valve, and by the time the chyme is below about pH 4, more than 60-90% of the meal has transitioned into the duodenum. Wild type lipase from B. cepacia (e.g., SEQ ID NO: 1), has good survivability down to pH 4. However, there may be brief periods when a lipase may be subjected to pH levels less than pH 4.0. Therefore, it may be desirable for a recombinant mutant lipase to exhibit improved stability down to about pH 3.0 to 3.5. Because a recombinant mutant lipase, can, in certain embodiments, be taken with food, improved stability at the very low pH of the fasted-state stomach may not be required.

In certain embodiments, the lipase has a half-life of at least about 35 minutes, at least about 50 minutes, at least about 75 minutes, at least about 100 minutes, at least about 125 minutes, at least about 130 minutes, at least about 135 minutes, at least about 140 minutes, at least about 145 minutes, or at least about 150 minutes at about pH 3.0. For example, in certain embodiments, the lipase has a half-life of from about 50 minutes to about 200 minutes, for example, from about 50 minutes to about 100 minutes, from about 50 minutes to about 150 minutes, from about 50 minutes to about 175 minutes, from about 50 minutes to about 200 minutes, from about 75 minutes to about 100 minutes, from about 75 minutes to about 150 minutes, from about 75 minutes to about 175 minutes, from about 75 minutes to about 200 minutes, from about 100 minutes to about 150 minutes, from about 100 minutes to about 175 minutes, from about 100 minutes to about 200 minutes, from about 150 minutes to about 175 minutes, from about 150 minutes to about 200 minutes.

In certain embodiments, the lipase has at least 1.5 fold, 2 fold, 2.5 fold, 3 fold higher stability at about pH 3.0, compared to the corresponding wild-type lipase. For example, the lipase can have between about 1.5 fold and about 2 fold, about 1.5 fold and about 2.5 fold, about 1.5 fold and about 3 fold, about 1.5 fold and about 3.5 fold, about 2 fold and about 2.5 fold, about 2 fold and about 3 fold, about 2 fold and about 3.5 fold, about 2.5 fold and about 3 fold, about 2.5 fold and about 3.5 fold, about 3 fold and about 3.5 fold higher stability at about pH 3.0, compared to the corresponding wild-type lipase.

Methods for testing for the stability of a lipase are known in the art and can include, for example, the methods described in Example 3 herein. In certain embodiments, stability of a lipase in low pH is determined using by exposing the lipase to the specified pH (e.g., pH 3.0), adding p-NPP (p-nitrophenyl palmitate), and detecting the presence or amount of the p-NPP cleavage product p-nitrophenolate by colorimetric assay.

b. Recombinant Mutant Lipases with Increased Stability in the Presence of Proteases

In certain embodiments, the lipase has increased stability in the presence of a protease (e.g., a serine protease and/or an aspartic protease) relative to the corresponding wild-type microbial lipase enzyme. The recombinant mutant lipases described herein, in certain embodiments, are designed to be immediately available in the stomach and will be exposed to proteolytic enzymes in the stomach, such as pepsin and other proteases Increased stability in the presence of a protease allows a recombinant mutant lipase to survive the harsh conditions of the stomach.

In certain embodiments, the engineered lipase has increased stability in the presence of an aspartic acid (e.g., pepsin) relative to the corresponding wild-type lipase. Pepsin has maximum activity at low pH levels (pH 1.5 to 4). Accordingly, in certain embodiments, the engineered lipase also has increase stability at low pH (e.g., pH 3.8).

In certain embodiments, the lipase has a half-life of at least about 50 minutes, at least about 75 minutes, at least about 100 minutes, at least about 125 minutes, at least about 150 minutes, at least about 175 minutes, at least about 200 minutes, at least about 225 minutes, at least about 230 minutes, or at least about 235 minutes in the presence of an aspartic protease such as pepsin. In certain embodiments, the lipase has a half-life of between about 75 minutes and 100 minutes, between about 75 minutes and about 125 minutes, between about 75 minutes and about 150 minutes, between about 75 minutes and about 175 minutes, between about 75 minutes and about 200 minutes, between about 75 minutes and about 225 minutes, between about 75 minutes and about 230 minutes, between about 75 minutes about and about 235 minutes, between about 75 minutes and about 250 minutes, between about 100 minutes and about 125 minutes, between about 100 minutes and about 150 minutes, between about 100 minutes and about 175 minutes, between about 100 minutes and about 200 minutes, between about 100 minutes and about 225 minutes, between about 100 minutes and about 230 minutes, between about 100 minutes about and about 235 minutes, between about 100 minutes and about 250 minutes, between about 125 minutes and about 150 minutes, between about 125 minutes and about 175 minutes, between about 125 minutes and about 200 minutes, between about 125 minutes and about 225 minutes, between about 125 minutes and about 230 minutes, between about 125 minutes about and about 235 minutes, between about 125 minutes and about 250 minutes, between about 150 minutes and about 175 minutes, between about 150 minutes and about 200 minutes, between about 150 minutes and about 225 minutes, between about 150 minutes and about 230 minutes, between about 150 minutes about and about 235 minutes, between about 150 minutes and about 250 minutes, between about 175 minutes and about 200 minutes, between about 175 minutes and about 225 minutes, between about 175 minutes and about 230 minutes, between about 175 minutes about and about 235 minutes, between about 175 minutes and about 250 minutes, between about 200 minutes and about 225 minutes, between about 200 minutes and about 230 minutes, between about 200 minutes about and about 235 minutes, between about 200 minutes and about 250 minutes, between about 225 minutes and about 230 minutes, between about 225 minutes about and about 235 minutes, between about 225 minutes and about 250 minutes, or between about 235 minutes and 250 minutes in the presence of an aspartic protease such as pepsin. In certain embodiments, the pepsin is present at low pH (e.g., pH 3.6) typical of fed-state stomach.

Methods for testing for the stability of a lipase in the presence of an aspartic protease such as pepsin are known in the art and can include, for example, the methods described in Example 4 herein. In certain embodiments, stability of a lipase in the presence of an aspartic protease is determined using by exposing the lipase to the protease (e.g., pepsin), inactivating the pepsin, then adding p-NPP (p-nitrophenyl palmitate) and detecting the presence or amount of the p-NPP cleavage product p-nitrophenolate by colorimetric assay.

In certain embodiments, the lipase has at least 1.5 fold, 2 fold, 2.5 fold, 3 fold, 3.5 fold, or 4 fold higher stability in the presence of an aspartic protease (e.g., at pH 3.6), such as pepsin, compared to the corresponding wild-type lipase. In certain embodiments the lipase has between about 1.5 fold and about 2 fold, between about 1.5 fold and about 2.5 fold, between about 1.5 fold and about 3 fold, between about 1.5 fold and about 3.5 fold, between about 1.5 fold and about 4 fold, between about 2 fold and about 2.5 fold, between about 2 fold and about 3 fold, between about 2 fold and about 3.5 fold, between about 2 fold about 4 fold, between about 2.5 fold and about 3 fold, between about 2.5 fold and about 3.5 fold, between about 2.5 fold and about 4 fold, between about 3 fold and about 3.5 fold, between about 3 fold and about 3.5 fold, or between about 3.5 fold and about 4 fold higher stability in the presence of an aspartic protease (e.g., at pH 3.6), such as pepsin, compared to the corresponding wild-type lipase.

Further, in certain embodiments, an engineered lipase will be delivered in combination with a protease, for protein digestion, and an amylase, for starch digestion. Accordingly, in certain embodiments, the lipase is exposed to the protease from A. melleus for co-dosing. A. melleus protease is a serine protease with a maximum activity at pH 7 to pH 8 and a pH range of greater than 50% activity from pH 5 to pH 11. Unlike mammalian proteases such as trypsin and chymotrypsin which cleave proteins only after specific amino acids, the A. melleus protease (also called SAP or oryzin) cleaves proteins down to small oligomers and individual amino acids. The recombinant mutant lipases described herein are expected to be in the presence of the A. melleus protease for three to six hours (the transit time from the fed state stomach through the small intestine), so in certain embodiments, the engineered lipase is resistant to degradation by this protease.

In certain embodiments, the lipase has a half-life of at least 50 minutes, 75 minutes, 100 minutes, 125 minutes, 150 minutes, 175 minutes, 180 minutes, 185 minutes, 190 minutes, 195 minutes, or 200 minutes in the presence of a serine protease, such as A. melleus protease. For example, the lipase can have a half-life of between about 75 minutes and 100 minutes, between about 75 minutes and about 125 minutes, between about 75 minutes and about 150 minutes, between about 75 minutes and about 175 minutes, between about 75 minutes and about 200 minutes, between about 75 minutes and about 225 minutes, between about 100 minutes and about 125 minutes, between about 100 minutes and about 150 minutes, between about 100 minutes and about 175 minutes, between about 100 minutes and about 200 minutes, between about 100 minutes and about 225 minutes, between about 125 minutes and about 150 minutes, between about 125 minutes and about 175 minutes, between about 125 minutes and about 200 minutes, between about 125 minutes and about 225 minutes, between about 150 minutes and about 175 minutes, between about 150 minutes and about 200 minutes, between about 150 minutes and about 225 minutes, between about 175 minutes and about 200 minutes, between about 175 minutes and about 225 minutes, or between about 200 minutes and about 225 minutes, in the presence of a serine protease, such as A. melleus protease.

In certain embodiments, the lipase has at least 1.5 fold, 2 fold, 2.5 fold, 3 fold, 3.5 fold, or 4 fold higher stability in the presence of a serine protease, such as A. melleus protease, compared to the corresponding wild-type lipase. In certain embodiments the lipase has between about 1.5 fold and about 2 fold, between about 1.5 fold and about 2.5 fold, between about 1.5 fold and about 3 fold, between about 1.5 fold and about 3.5 fold, between about 1.5 fold and about 4 fold, between about 2 fold and about 2.5 fold, between about 2 fold and about 3 fold, between about 2 fold and about 3.5 fold, between about 2 fold about 4 fold, between about 2.5 fold and about 3 fold, between about 2.5 fold and about 3.5 fold, between about 2.5 fold and about 4 fold, between about 3 fold and about 3.5 fold, between about 3 fold and about 4 fold, or between about 3.5 fold and about 4 fold higher stability in the presence of a serine protease, such as A. melleus protease, compared to the corresponding wild-type lipase.

Methods for testing for the stability of a lipase in the presence of an aspartic protease such as pepsin are known in the art and can include, for example, the methods described in Example 5 herein. In certain embodiments, stability of a lipase in the presence of an aspartic protease is determined by exposing the lipase to the protease (e.g., pepsin), inactivating the pepsin, then adding p-NPP (p-nitrophenyl palmitate) and detecting the presence or amount of the p-NPP cleavage product p-nitrophenolate by colorimetric assay.

In certain embodiments, the recombinant mutant lipase is administered in combination with pancreatin. Pancreatin contains up to 20 different enzymes, with three main enzyme classes as active ingredients: amylase, lipase, and a protease. The pancreatin proteases include trypsin, chymotrypsin, elastase, carboxypeptidase A and carboxypeptidase B. Pancreatin and pancreatin-based preparations such as pancrelipase are currently used to manage exocrine pancreatic insufficiency. Accordingly, in certain embodiments, the lipase is exposed to the proteases in pancreatin for co-dosing. Thus, the engineered lipase may be resistant to degradation by pancreatin.

In certain embodiments, the lipase has a half-life of at least 50 minutes, 75 minutes, 100 minutes, 125 minutes, 150 minutes, 175 minutes, 180 minutes, 185 minutes, 190 minutes, 195 minutes, or 200 minutes in the presence of pancreatin. For example, the lipase can have a half-life of between about 75 minutes and 100 minutes, between about 75 minutes and about 125 minutes, between about 75 minutes and about 150 minutes, between about 75 minutes and about 175 minutes, between about 75 minutes and about 200 minutes, between about 75 minutes and about 225 minutes, between about 100 minutes and about 125 minutes, between about 100 minutes and about 150 minutes, between about 100 minutes and about 175 minutes, between about 100 minutes and about 200 minutes, between about 100 minutes and about 225 minutes, between about 125 minutes and about 150 minutes, between about 125 minutes and about 175 minutes, between about 125 minutes and about 200 minutes, between about 125 minutes and about 225 minutes, between about 150 minutes and about 175 minutes, between about 150 minutes and about 200 minutes, between about 150 minutes and about 225 minutes, between about 175 minutes and about 200 minutes, between about 175 minutes and about 225 minutes, or between about 200 minutes and about 225 minutes, in the presence of pancreatin.

In certain embodiments, the lipase has at least 1.5 fold, 2 fold, 2.5 fold, 3 fold, 3.5 fold, or 4 fold higher stability in the presence of pancreatin, compared to the corresponding wild-type lipase. In certain embodiments the lipase has between about 1.5 fold and about 2 fold, between about 1.5 fold and about 2.5 fold, between about 1.5 fold and about 3 fold, between about 1.5 fold and about 3.5 fold, between about 1.5 fold and about 4 fold, between about 2 fold and about 2.5 fold, between about 2 fold and about 3 fold, between about 2 fold and about 3.5 fold, between about 2 fold about 4 fold, between about 2.5 fold and about 3 fold, between about 2.5 fold and about 3.5 fold, between about 2.5 fold and about 4 fold, between about 3 fold and about 3.5 fold, between about 3 fold and about 4 fold, or between about 3.5 fold and about 4 fold higher stability in the presence of a pancreatin compared to the corresponding wild-type lipase.

Methods for testing for the stability of a lipase in the presence of pancreatin are known in the art. In certain embodiments, stability of a lipase in the presence of pancreatin is determined using by exposing the lipase to pancreatin, optionally inactivating the proteases in pancreatin, then adding p-NPP (p-nitrophenyl palmitate) and detecting the amount of the p-NPP cleavage product p-nitrophenolate by colorimetric assay. The stability is determined by comparing the amount of p-NPP cleavage to a control sample of the lipase that is not treated with pancreatin.

c. Recombinant Mutant Lipases can have Increased Lipase Activity

Dietary lipids, including long-chain polyunsaturated fats (LCPUFAs), such as DHA, EPA, and AA, are primarily in the form of long-chain triglycerides. Long-chain triglycerides are made of three long-chain fatty acids bound to a glycerol molecule via ester linkages. Absorption of long-chain triglycerides by the body first requires the enzymatic action of lipase, e.g., pancreatic lipase, which digests triglycerides through hydrolysis, breaking them down into one sn-2 monoglyceride and two free fatty acids. The term “free fatty acids”, i.e., fatty acids not attached to other molecules (such as a glycerol backbone), is used to refer to the byproducts of fat digestion. The terms “digestion” and “hydrolysis” are used interchangeably to refer to the enzymatic action of lipase to breakdown a lipid triglyceride into a monoglyceride and free fatty acids. The hydrolysis products, monoglycerides and free fatty acids, are then used as energy and absorbed into enterocytes, largely by passive diffusion. Once free fatty acids and monoglycerides are absorbed, they are transported to the liver and ultimately to tissues in the body for various physiological purposes.

Additionally, the chain lengths and the number of carbon-carbon double bonds of fatty acids may influence fat absorption. Dietary fatty acids found in food are long-chain fatty acids having at least 12 carbons, for example 16, 18, or 20 carbons, known as C16, C18, and C20 long-chain fatty acids. Medium-chain fatty acids having less than or equal to 12 carbons, for example, 8 and 12 carbons, known as C8 and C12 are generally not found in food (except for coconuts) and are thus less important for digestion and absorption in humans. Short-chain fatty acids having less than or equal to a few carbons, for example, 2, 3, and 4 carbons, known as C2, C3, and C4, are the major anions found the stool, but are not found in food. Short-chain fatty acids result from digestion by the bacteria in the colon.

While all fats provide caloric benefit, they have different impacts on physiological functions. Short-chain triglycerides (SCTs) and medium-chain triglycerides (MCTs) are absorbed directly through the villi of the intestinal mucosa. MCTs can be readily absorbed due to their shorter chain lengths and the residual activity of gastric lipase, even in patients having compromised pancreatic output or pancreatic insufficiency. Long-chain triglycerides (LCTs) are not directly absorbed but instead must first be hydrolyzed into free fatty acids and monoglycerides by pancreatic lipase before they are absorbed in the small intestine. Once free fatty acids and monoglycerides are absorbed, they are transported to the liver and ultimately to tissues in the body for various physiological purposes. While both LCTs and MCTs provide calories, only LCTs, specifically LCPUFAs, provide structural components of membranes and biological mediators involved in the regulation of many physiological functions. MCTs, when substituted for LCTs, have been shown to increase energy expenditure and satiety, leading to reduced overall caloric intake and reduced body fat mass. This makes MCTs a poor long-term energy source for patients having compromised pancreatic output or pancreatic insufficiency.

In certain embodiments, a recombinant mutant lipase described herein has at least 0.5 fold, 1 fold, 1.5 fold, 2 fold, 2.5 fold, or 3 fold higher activity (e.g., at about pH 3.0), compared to the corresponding wild-type lipase. In certain embodiments the lipase has between about 1.5 fold and about 2 fold, between about 1.5 fold and about 2.5 fold, between about 1.5 fold and about 3 fold, between about 1.5 fold and about 3.5 fold, between about 2 fold and about 2.5 fold, between about 2 fold and about 3 fold, between about 2 fold and about 3.5 fold, between about 2.5 fold and about 3 fold, between about 2.5 fold and about 3.5 fold, or between about 3 fold and about 3.5 fold higher activity (e.g., at about pH 3.0) than a corresponding wild type lipase.

In certain embodiments, the lipase preferentially hydrolyzes the sn-1 and sn-3 positions on a triglyceride. In certain embodiments, the lipase enzymatic activity (e.g., specific activity) is not inhibited by bile salts. In certain embodiments, the lipase does not require a colipase.

In certain embodiments, the lipase has a specific activity at pH 3.0 of at least 300, 400, 500, 600, 700, 800, 900, or 1,000 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3). In certain embodiments, the lipase has a specific activity at pH 3.0 of between about 300 and about 400, between about 300 and about 500, between about 300 and about 600, between about 300 and about 700, between about 300 and about 800, between about 300 and about 900, between about 300 and about 1,000, between about 300 and about 1,100, between about 400 and about 500, between about 400 and about 600, between about 400 and about 700, between about 400 and about 800, between about 400 and about 900, between about 400 and about 1,000, between about 400 and about 1,100, between about 500 and about 600, between about 500 and about 700, between about 500 and about 800, between about 500 and about 900, between about 500 and about 1,000, between about 500 and about 1,100, between about 600 and about 700, between about 600 and about 800, between about 600 and about 900, between about 600 and about 1,000, between about 600 and about 1,100, between about 700 and about 800, between about 700 and about 900, between about 700 and about 1,000, between about 700 and about 1,100, between about 800 and about 900, between about 800 and about 1,000, between about 800 and about 1,100, between about 900 and about 1,000, between about 900 and about 1,100, or between about 1,000 and about 1,100 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3).

In certain embodiments, the lipase has a specific activity at pH 4.0, pH 5.0, or pH 6.0 of at least 600, 700, 800, 900, 1,000, 1,100, 1,200, 1,300, 1,400, 1,500, or 2,000 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3). In certain embodiments, the lipase has a specific activity at pH 4.0, pH 5.0, or pH 6.0 of between about 600 and about 700, between about 600 and about 800, between about 600 and about 900, between about 600 and about 1,000, between about 600 and about 1,100, between about 600 and about 1,200, between about 600 and about 1,300, between about 600 and about 1,400, between about 600 and about 1,500, between about 600 and about 2,000, between about 700 and about 800, between about 700 and about 900, between about 700 and about 1,000, between about 700 and about 1,100, between about 700 and about 1,200, between about 700 and about 1,300, between about 700 and about 1,400, between about 700 and about 1,500, between about 700 and about 2,000, between about 800 and about 900, between about 800 and about 1,000, between about 800 and about 1,100, between about 800 and about 1,200, between about 800 and about 1,300, between about 800 and about 1,400, between about 800 and about 1,500, between about 800 and about 2,000, between about 900 and about 1,000, between about 900 and about 1,100, between about 900 and about 1,200, between about 900 and about 1,300, between about 900 and about 1,400, between about 900 and about 1,500, between about 900 and about 2,000, between about 1,000 and about 1,100, between about 1,000 and about 1,200, between about 1,000 and about 1,300, between about 1,000 and about 1,400, between about 1,000 and about 1,500, between about 1,000 and about 2,000, between about 1,100 and about 1,200, between about 1,100 and about 1,300, between about 1,100 and about 1,400, between about 1,100 and about 1,500, between about 1,100 and about 2,000, between about 1,200 and about 1,300, between about 1,200 and about 1,400, between about 1,200 and about 1,500, between about 1,200 and about 2,000, between about 1,300 and about 1,400, between about 1,300 and about 1,500, between about 1,300 and about 2,000, between about 1,400 and about 1,500, between about 1,400 and about 2,000, or between about 1,500 and 2,000 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3).

In certain embodiments, the lipase has a specific activity at pH 7.0 of at least 1,000, 1,100, 1,200, 1,300, 1,400, 1,500, or 2,000 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3). In certain embodiments, the lipase has a specific activity at pH 7.0 of between about 1,000 and about 1,100, between about 1,000 and about 1,200, between about 1,000 and about 1,300, between about 1,000 and about 1,400, between about 1,000 and about 1,500, between about 1,000 and about 2,000, between about 1,100 and about 1,200, between about 1,100 and about 1,300, between about 1,100 and about 1,400, between about 1,100 and about 1,500, between about 1,100 and about 2,000, between about 1,200 and about 1,300, between about 1,200 and about 1,400, between about 1,200 and about 1,500, between about 1,200 and about 2,000, between about 1,300 and about 1,400, between about 1,300 and about 1,500, between about 1,300 and about 2,000, between about 1,400 and about 1,500, between about 1,400 and about 2,000, or between about 1,500 and 2,000 μmol fatty acids (FA) produced/min/mg of lipase towards a long-chain triglyceride substrate including 37% DHA triglyceride and 22% oleic acid triglyceride or triolein (for example, an exemplary long chain triglyceride substrate set forth in TABLE 3).

In certain embodiments, the lipase has at least 60%, 70%, 80%, 85%, 90%, 95%, 96%, 97%, 98%, 99%, or 100% of the enzymatic activity of the corresponding wild-type microbial lipase enzyme. In certain embodiments, the lipase has between about 60% and about 70%, between about 60% and about 80%, between about 60% and about 85%, between about 60% and about 90%, between about 60% and about 95%, between about 60% and about 96%, between about 60% and about 97%, between about 60% and about 98%, between about 60% and about 99%, between about 60% and about 100%, between about 70% and about 80%, between about 70% and about 85%, between about 70% and about 90%, between about 70% and about 95%, between about 70% and about 96%, between about 70% and about 97%, between about 70% and about 98%, between about 70% and about 99%, between about 70% and about 100%, between about 80% and about 85%, between about 80% and about 90%, between about 80% and about 95%, between about 80% and about 96%, between about 80% and about 97%, between about 80% and about 98%, between about 80% and about 99%, between about 80% and about 100%, between about 85% and about 90%, between about 85% and about 95%, between about 85% and about 96%, between about 85% and about 97%, between about 85% and about 98%, between about 85% and about 99%, between about 85% and about 100%, between about 90% and about 95%, between about 90% and about 96%, between about 90% and about 97%, between about 90% and about 98%, between about 90% and about 99%, between about 90% and about 100%, between about 95% and about 96%, between about 95% and about 97%, between about 95% and about 98%, between about 95% and about 99%, between about 95% and about 100%, between about between about 96% and about 97%, between about 96% and about 98%, between about 96% and about 99%, between about 96% and about 100%, between about 97% and about 98%, between about 97% and about 99%, between about 97% and about 100%, between about 98% and about 99%, between about 98% and about 100%, or between about 99% and about 100% of the enzymatic activity of the corresponding wild-type microbial lipase enzyme.

In certain embodiments, the lipase remains sufficiently active at a pH in the range of 3.5 to 7.0 to hydrolyze long-chain poly-unsaturated fatty acids (LCPUFAs), e.g., DHA and EPA, or long-chain triglycerides, e.g., oleic acid or triolein, in the gastrointestinal tract of a subject. In certain embodiments, the lipase is at least 2 fold, 10 fold, 100 fold or 1000 fold more active than pancrelipase when tested under the same conditions.

In certain embodiments, more than 50%, 60%, 70%, 80%, or 90% of the lipase remains active in the fed-state stomach of a subject for 60-120 minutes. In certain embodiments, between about 50% and about 60%, between about 50% and about 70%, between about 50% and about 80%, between about 50% and about 90%, between about 60% and about 70%, between about 60% and about 80%, between about 60% and about 90%, between about 70% and about 80%, or between about 70% and about 90%, or between about 80% and about 90% of the lipase remains active in the fed-state stomach of a subject for 60-120 minutes.

In certain embodiments, the lipase digests greater than 20%, 30%, 40%, or 50% of ingested fats in the stomach of a subject to fatty acids and monoglycerides. In certain embodiments, the lipase digests between about 20% and about 30%, between about 20% and about 40%, between about 20% and about 50%, between about 30% and about 40%, between about 30% and about 50%, or between about 40% and about 50% of ingested fats in the stomach of a subject to fatty acids and monoglycerides.

In certain embodiments, more than 50%, 60%, 70%, 80%, or 90% of the lipase remains active through the small intestine of a subject from about 240 to about 360 minutes. In certain embodiments, between about 50% and about 60%, between about 50% and about 70%, between about 50% and about 80%, between about 50% and about 90%, between about 60% and about 70%, between about 60% and about 80%, between about 60% and about 90%, between about 70% and about 80%, or between about 70% and about 90%, or between about 80% and about 90% of the lipase remains active through the small intestine of a subject from about 240 to about 360 minutes.

In certain embodiments, it may be desirable for the activity of the lipase to diminish in the large intestine. Accordingly, in certain embodiments, the lipase has reduced activity in the large intestine after 10 hours, 12 hours or 18 hours. In certain embodiments, the lipase is able to digest less than 50%, less than 60% or less than 70% or less than 80% or less than 90% of remaining fat in the large intestine.

In certain embodiments, the lipase digests greater than 50%, 60%, 70%, 80%, or 90% of ingested fats in the small intestine of a subject to fatty acids and monoglycerides. In certain embodiments, the lipase digests between about 50% and about 60%, between about 50% and about 70%, between about 50% and about 80%, between about 50% and about 90%, between about 60% and about 70%, between about 60% and about 80%, between about 60% and about 90%, between about 70% and about 80%, or between about 70% and about 90%, or between about 80% and about 90% of ingested fats in the small intestine of a subject to fatty acids and monoglycerides.

In certain embodiments, the lipase increases absorption of long-chain unsaturated fatty acids in the plasma in a subject within 30 minutes, 45 minutes, 60 minutes, 90 minutes, or 120 minutes by more than 25%, 35%, 50%, 100%, or 200% relative to the same subject when that subject has not been administered the lipase, or relative to a similar subject that has not been administered the lipase. In certain embodiments, the lipase increases absorption of fat-soluble vitamins (e.g., vitamin A, vitamin D, vitamin E, vitamin K). In certain embodiments, the lipase increases absorption of choline.

Fat hydrolysis by a lipase can by assayed using any method known in the art. For example, a modified quantitative colorimetric assay (Abcam® Free Fatty Acid Quantification Kit) can be used to measure the amount of free fatty acids using a given lipid substrate. Fats that are more complex (for example, fats that have a longer chain length and larger number of double bonds) are more challenging for lipases to hydrolyze into free fatty acids and monoglycerides. One such complex fat, DHA, is a relevant surrogate for overall fat hydrolysis or digestion, because it has longer carbon chains and more double bonds relative to other fats or LCPUFAs, so it is more difficult to hydrolyze. Accordingly, assaying DHA hydrolysis is a useful surrogate for lipase's ability to digest all triglycerides. Through the course of experiments the substrate DHA from oil is in the form of a triglyceride while the product measured in the method is the DHA free fatty acid form.

In one embodiment, lipase activity can be measured using a DHA hydrolysis assay using an oil containing ˜37% DHA triglycerides and ˜22% oleic acid triglycerides, with the remainder being mostly comprised of myristic acid triglyceride, palmitic acid triglyceride, stearic acid triglyceride and lauric acid triglyceride as well as palmitoleic acid triglyceride. (NuCheck, Elysian MN.) The major components of such a DHA triglyceride oil are shown in TABLE 3.

TABLE 3
Major Components of DHA triglyceride oil

| | | Percent of Total |
|---|---|---|
| Triglyceride | Chain Length | Triglycerides |
| DHA triglyceride | c22, 6 double-bonds | 37% |
| Oleic acid triglyceride | c18, 1 double-bond | 22% |
| Myristic acid triglyceride | c14, saturated | 15% |
| Palmitic acid triglyceride | c16, saturated | 13% |
| Lauric acid triglyceride | C12, saturated | 6% |
| Palmitoleic acid triglyceride | C 16, 1 double bond | 3% |
| Linoleic acid triglyceride | C 18, 2 double bonds | 1.2% |
| Stearic acid triglyceride | C18 saturated | 0.76% |
| Nervonic acid triglyceride | C 24, 1 double bond | 0.55% |
| Other | — | ~1% |

Oleic acid triglyceride is a fat substrate with three fatty acids (18-carbons) attached to a glycerol backbone and contains 1 double-bond. Oleic acid triglyceride is a common dietary fat and is present in olive oil in an percentage between about 55% and 83%. The triglyceride of oleic acid is hydrolyzed by pancreatic lipases to form two oleic acid fatty acids and an sn-2 monoglyceride. Like DHA triglyceride, oleic acid triglyceride can serve as a surrogate for overall dietary fat hydrolysis.

Triolein is the purified form of oleic acid in the triglyceride form. Since olive oil varies from lot to lot, use of olive oil in hydrolysis assays can result in inconsistent measurements. Accordingly, triolein can be used for assessing the ability of lipase to hydrolyze oleic acid in the triglyceride form and may provide more consistent results as compared to olive oil.

In a certain embodiment, a lipase potency assay is used to measure release of fatty acids from triglycerides by a four-step process: 1) hydrolysis of the triglycerides at pH 6 to release free fatty acids (FFAs), 2) conjugation of FFAs to Coenzyme A, 3) oxidation of the FFA-Coenzyme A complex to generate hydrogen peroxide and 4) detection of the peroxide using a colorimetric oxidation dye. The amount of colorimetric dye produced is proportional to the amount of FFAs released by lipase, and the specific activity of the lipase is defined as the amount of enzyme needed to convert 1 μmole of substrate per minute. The assay is explained in further detail in Example 2 herein.

It is contemplated that a disclosed recombinant mutant lipase may be modified, engineered or chemically conjugated. For example, it is contemplated that a disclosed recombinant mutant lipase can be conjugated to an effector agent using standard in vitro conjugation chemistries. If the effector agent is a polypeptide, the lipase can be chemically conjugated to the effector or joined to the effector as a fusion protein. Construction of fusion proteins is within ordinary skill in the art.

### III. Lipase Production

Methods for producing lipase enzymes of the invention are known in the art. For example, DNA molecules encoding a lipase can be chemically synthesized using the sequence information provided herein. Synthetic DNA molecules can be ligated to other appropriate nucleotide sequences, including, e.g., expression control sequences, to produce conventional gene expression constructs encoding the desired lipase.

Nucleic acids encoding desired lipases can be incorporated (ligated) into expression vectors, which can be introduced into host cells through conventional transfection or transformation techniques. Transformed host cells can be grown under conditions that permit the host cells to express the genes that encode the lipase enzyme.

Nucleic acids encoding recombinant mutant lipases of the invention may be generated by mutating a nucleotide sequence encoding the wild type B. cepacia lipase, e.g., SEQ ID NO: 1 disclosed herein, using methods known in the art. Furthermore, in certain embodiments, nucleic acids encoding recombinant mutant B. cepacia lipases of the invention may be codon optimized for expression in a heterologous cell, e.g., a B. cepacia cell, a Burkholderia glumae cell, a Pseudomonas fluorescens cell, a Chromobacterium viscosum cell, a Pseudomonas luteola cell, a Pseudomonas fragi cell, or a Escherichia coli cell, using methods known in the art.

In certain embodiments, the disclosure relates to a cell comprising an expression vector as described herein. In certain embodiments, the cell is a B. cepacia, Burkholderia glumae, Pseudomonas fluorescens, Chromobacterium viscosum, Pseudomonas luteola, Pseudomonas fragi, or Escherichia coli cell.

In one embodiment, the disclosure relates to an exemplary nucleotide sequence encoding a recombinant mutant lipase. In certain embodiments, the nucleotide sequence encoding a recombinant mutant lipase comprises nucleotide substitutions as compared to a wild-type lipase, e.g., a wild-type B. cepacia lipase. Wild-type nucleic acids encoding a B. cepacia lipase are known in the art and include, for example, the following sequence, SEQ ID NO: 39:

GCCGACAACT ACGCGGCGAC GCGTTATCCG ATCATTCTCG
TGCACGGGCT CACGGGCACC GACAAATACG CAGGTGTGCT
CGAGTACTGG TACGGGATCC AGGAGGACCT GCAGCAGCGT
GGCGCGACCG TCTATGTCGC TAACCTGTCG GGCTTCCAGA
GCGACGACGG CCCGAACGGG CGCGGCGAAC AGTTGCTGGC
CTACGTGAAG ACGGTGCTCG CCGCGACGGG GGCGACCAAG
GTCAACCTCG TCGGCCACAG CCAGGGCGGG CTGACGTCGC
GCTATGTCGC GGCCGTCGCG CCCGATCTGG TCGCGTCGGT
GACGACGATC GGCACGCCGC ATCGCGGCTC CGAGTTCGCC
GACTTCGTGC AGGGCGTGCT CGCGTACGAT CCGACCGGGC
TGTCGTCGAC GGTGATCGCC GCGTTCGTCA ATGTGTTCGG
AATCCTCACG AGCAGCAGCA ACAACACGAA CCAGGACGCG
CTCGCGGCGC TGAAGACGCT GACGACCGCG CAGGCCGCCA
CGTACAACCA GAACTACCCT AGCGCGGGCC TCGGCGCGCC
GGGCAGTTGC CAGACCGGCG CGCCGACGGA AACCGTCGGC
GGCAACACGC ATCTGCTGTA TTCGTGGGCC GGCACGGCGA
TCCAGCCGAC GATCTCCGTG TTCGGCGTCA CGGGTGCGAC
GGATACGAGC ACCATTCCGC TCGTCGATCC GGCGAACGCG
CTCGACCCGT CGACGCTCGC GCTGTTCGGC ACCGGCACGG
TGATGGTCAA CCGCGGTTCG GGCCAGAACG ACGGGGTCGT
GTCGAAGTGC AGCGCGCTGT ACGGCCAGGT GCTGAGCACG
AGCTACAAGT GGAACCATCT CGACGAGATC AACCAGTTGC
TCGGCGTGCG CGGCGCGAAT GCGGAAGATC CGGTCGCGGT
GATCCGCACG CATGCGAACC GGCTGAAGCT CGCGGGCGTG

In one embodiment, the disclosure relates to an exemplary nucleotide sequence encoding a recombinant mutant lipase that comprises the following substitutions: D102Q, N154H, and F221L, e.g., a nucleotide sequence encoding a recombinant mutant B. cepacia lipase referred to as V130 herein.

In one embodiment, the disclosure relates to an exemplary nucleotide sequence encoding a recombinant mutant lipase that comprises the following substitutions: D102Q, G125S, N154H, F221L, V266L, and N300Y, e.g., a nucleotide sequence encoding a recombinant mutant B. cepacia lipase referred to as V290 herein.

In one embodiment, the disclosure relates to an exemplary nucleotide sequence encoding a recombinant mutant lipase that comprises the following substitutions: T79Q, D102Q, G125S, T137A, N154H, F221L, F249L, V266L, N300Y, and T227K, e.g., a nucleotide sequence encoding a recombinant mutant lipase referred to as V309 herein.

In one embodiment, the disclosure relates to an exemplary nucleotide sequence encoding a recombinant mutant lipase that comprises the following substitutions: T79Q, D102Q, G125S, T137A, N154H, F221L, V266L, S281A, N300Y, and T227K, e.g., a nucleotide sequence encoding a recombinant mutant lipase referred to as V311 herein.

In one embodiment, the disclosure relates to an exemplary nucleotide sequence encoding a recombinant mutant lipase that comprises the following substitutions: T79Q, D102Q, G125S, S153N, N154H, F221L, V266L, S281A, N300Y, and T227K, e.g., a nucleotide sequence encoding a recombinant mutant lipase referred to as V317 herein.

In one embodiment, the disclosure relates to an exemplary nucleotide sequence encoding a recombinant mutant lipase that comprises the following substitutions: T79Q, D102Q, G125S, S153N, N154H, F221L, F249L, V266L, N300Y, and G250A, e.g., a nucleotide sequence encoding a recombinant mutant lipase referred to as V318 herein.

In one embodiment, the disclosure relates to an exemplary nucleotide sequence encoding a recombinant mutant lipase that comprises the following substitutions: T79Q, D102Q, G125S, S153N, N154H, F221L, F249L, V266L, S281A, and N300Y, e.g., a nucleotide sequence encoding a recombinant mutant lipase referred to as V319 herein.

In one embodiment, the disclosure relates to an exemplary nucleotide sequence encoding a recombinant mutant lipase that comprises the following substitutions: T79Q, D102Q, G125S, N154H, F221L, F249L, V266L, S281A, N300Y, and T227K, e.g., a nucleotide sequence encoding a recombinant mutant lipase referred to as V322 herein.

In one embodiment, the disclosure relates to an exemplary nucleotide sequence encoding a recombinant mutant lipase that comprises the following substitutions: D102Q, G125S, T137A, S153N, N154H, F221L, F249L, V266L, N300Y, and T227K, e.g., a nucleotide sequence encoding a recombinant mutant lipase referred to as V325 herein.

In one embodiment, the disclosure relates to an exemplary nucleotide sequence encoding a recombinant mutant lipase that comprises the following substitutions: D102Q, G125S, T137A, S153N, N154H, F221L, V266L, N300Y, T227K, and G250A, e.g., a nucleotide sequence encoding a recombinant mutant lipase referred to as V326 herein.

In one embodiment, the disclosure relates to an exemplary nucleotide sequence encoding a recombinant mutant lipase that comprises the following substitutions: D102Q, G125S, T137A, N154H, F221L, V266L, S281A, N300Y, T227K, and G250A, e.g., a nucleotide sequence encoding a recombinant mutant lipase referred to as V333 herein.

In one embodiment, the disclosure relates to an exemplary nucleotide sequence encoding a recombinant mutant lipase that comprises the following substitutions: D102Q, G125S, S153N, N154H, F221L, F249L, V266L, N300Y, T227K, and G250A, e.g., a nucleotide sequence encoding a recombinant mutant lipase referred to as V335 herein.

In one embodiment, the disclosure relates to an exemplary nucleotide sequence encoding a recombinant mutant lipase that comprises the following substitutions: D102Q, G125S, S153N, N154H, F221L, F249L, V266L, S281A, N300Y, and T227K, e.g., a nucleotide sequence encoding a recombinant mutant lipase referred to as V336 herein.

Specific expression and purification conditions will vary depending upon the expression system employed. For example, if a gene is to be expressed in E. coli, it can be cloned into an expression vector by positioning the engineered gene downstream from a suitable bacterial promoter, and a prokaryotic signal sequence. The expressed secreted protein is targeted to accumulate in the periplasmic space where it is harvested by osmotic shock or by disruption of the cells by French press or sonication. The refractile bodies then are solubilized, and the proteins refolded and cleaved by methods known in the art.

A lipase can be produced by growing (culturing) a host cell transfected with an expression vector encoding such lipase, under conditions that permit expression of the lipase. Following expression, the lipase can be harvested and purified or isolated using techniques known in the art, e.g., affinity tags such as glutathione-S-transferase (GST) and histidine tags. An exemplary expression and purification protocol for a lipase is described in Liu et al. (2011) APPL. MICROBIOL. BIOTECHNOL. 92(3):529-37.

### IV. Pharmaceutical Compositions and Dosages

For therapeutic use, a recombinant lipase described herein preferably is combined with a pharmaceutically acceptable carrier and/or an excipient. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Adeboye Adejare, Remington: The Science and Practice of Pharmacy (23rd ed. 2020). Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, the lipases can be formulated, or co-administered (either at the same time or sequentially), for example, by an enteral route (e.g., orally), with a pH increasing agent, for example, a protein pump inhibitor (PPI), to enhance the stability of the lipase, for example, in an acidic environment, for example, in the gastrointestinal tract.

Proton pump inhibitors are a group of drugs whose main action is pronounced and long-lasting reduction of gastric acid production. Proton pump inhibitors act by blocking the hydrogen/potassium adenosine triphosphatase enzyme system (the H⁺/K⁺ ATPase, or more commonly just gastric proton pump) of the gastric parietal cell. The proton pump is the terminal stage in gastric acid secretion, being directly responsible for secreting H⁺ ions into the gastric lumen, making it an ideal target for inhibiting acid secretion. Examples of proton pump inhibitors include: Omeprazole (brand names: LOSEC®, PRILOSEC®, ZEGERID®); Lansoprazole (brand names: PREVACID®, ZOTON®, INHIBITOL®); Esomeprazole (brand names: NEXIUM®); and Pantoprazole (brand names: PROTONIX®, SOMAC®, PANTOLOC®).

In certain embodiments, the lipases can be formulated, or co-administered (either at the same time or sequentially), for example, with a microbial protease, and/or a microbial amylase. Amylase hydrolyses α-1,4-glucosidic linkages of starch, glycogen and polysaccharides to produce a mixture of maltose and glucose. In certain embodiments, the protease is an A. melleus protease and/or the amylase is an A. oryzae amylase. In certain embodiments, the composition is formulated as an oral dosage form. In certain embodiments, the composition is a formulated as a powder, granulate, pellet, micropellet, liquid, or a tablet. In certain embodiments, the composition is encapsulated in a capsule or formulated as a tablet dosage form. In certain embodiments, the composition does not comprise an enteric coating.

Pharmaceutical compositions containing a recombinant lipase disclosed herein can be presented in a dosage unit form and can be prepared by any suitable method. A pharmaceutical composition should be formulated to be compatible with its intended route of administration, e.g., oral administration. The pharmaceutical compositions may be in a variety of forms. These include, for example, liquid, semi-solid and solid dosage forms, such as liquid solutions, dispersions or suspensions, tablets, pills, powders, liposomes and suppositories. The preferred form will depend upon the intended mode of administration and therapeutic application.

The composition can be formulated as a solution, microemulsion, dispersion, liposome, or other ordered structure suitable for stable storage at high concentration. Sterile solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants.

The amount of the lipase to be administered to the subject will depend upon a number of variables including, for example, the meal content and amount of fat ingested or the type of fat ingested, as well as the age, weight, gender, health, or disease or disorder associated with reduced ability to digest and/or absorb triglycerides that a given subject may have. Exemplary doses may include less than 400, 600, 800, or 1,000 mg of the lipase or pharmaceutical composition per day. The total units of lipase per meal can be about 10,000, 20,000, 50,000, 100,000, 200,000, 400,000 or more.

### V. Therapeutic Uses

The invention provides a method of treating a disease or disorder associated with an elevated amount of undigested lipid in a subject. In certain embodiments, the disease or disorder is associated with an elevated amount of undigested lipid in the gastrointestinal tract of the subject. The method comprises administering to the subject an effective amount of a disclosed recombinant lipase, either alone or in a combination with another therapeutic agent to treat the disease or disorder in the subject. The term “effective amount” as used herein refers to the amount of an active agent (e.g., a recombinant lipase of the present invention) sufficient to effect beneficial or desired results such as improved uptake or fatty acids in plasma and tissues or reduced undigested fat in the small intestine. An effective amount can be administered in one or more administrations, applications or dosages and is not intended to be limited to a particular formulation or administration route.

In certain embodiments, the method comprises orally administering to the subject an effective amount of a disclosed recombinant lipase, either alone or in a combination with another therapeutic agent to treat the disease or disorder in the subject.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. The term “treating” can also include ameliorating a symptom of the disease in the subject. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

Examples of diseases or disorders associated with an elevated amount of undigested lipid include those in which the subject exhibits low level secretion of pancreatic enzymes or has a physiological condition that affects fat hydrolysis or fat absorption (e.g., reduced gastric, duodenal, liver, bile, or gallbladder function); reduced gastrointestinal transit, motility, mixing, emptying; or reduced intestinal mucosa function (e.g., induced by mucosal damage) that results in fat maldigestion or fat malabsorption or a fatty acid deficiency. For example, such diseases and disorders may include exocrine pancreatic insufficiency (EPI), malabsorption syndrome, cystic fibrosis, chronic pancreatitis, acute pancreatitis, Schwachman-Diamond syndrome, a fatty acid disorder, Familial lipoprotein lipase deficiency, Johanson-Blizzard syndrome, Zollinger-Ellison syndrome, Pearson marrow syndrome, short-bowel syndrome, liver disease, primary biliary atresia, cholestasis, celiac disease, fatty liver disease, pancreatitis, diabetes, aging, cancer of the pancreas, stomach, small intestine, colon, rectal/anal, liver, hepatic, gallbladder, or, esophagus, cachexia, or a gastrointestinal disorder (e.g., Crohn's disease, irritable bowel syndrome, or ulcerative colitis), surgical invention of the stomach, small intestine, liver, gallbladder and pancreas. Other subjects suitable for treatment with the methods and compositions described herein are infants and those in critical care, who have an increased likelihood of exhibiting maldigestion or malabsorption of lipids.

In another embodiment, the disclosure relates to a method of improving the absorption of fatty acids in a subject in need thereof, the method comprising administering to the subject an effective amount of a lipase or a pharmaceutical composition as described herein, thereby improving absorption of fatty acids in the subject.

In another embodiment, the disclosure relates to a method of increasing the amount of fatty acids in plasma, erythrocytes, or a tissue of a subject in need thereof, the method comprising administering to the subject an effective amount of a lipase or a pharmaceutical composition as described herein, thereby increasing the amount of fatty acids in the subject.

In another embodiment, the disclosure relates to a method of increasing the ratio of omega-3 to omega-6 fatty acids in plasma, erythrocytes, or a tissue of a subject in need thereof, the method comprising administering to the subject an effective amount of a lipase or a pharmaceutical composition as described herein, thereby increasing the amount of fatty acids in the subject.

In another embodiment, the disclosure relates to a method of reducing the amount of fatty acids in the stool of a subject in need thereof, the method comprising administering to the subject an effective amount of a lipase or a pharmaceutical composition as described herein, thereby reducing the amount of fatty acids in the stool of the subject. In certain embodiments, the fatty acids are long-chain poly-unsaturated fatty acids (LCPUFAs). In certain embodiments, the fatty acids are omega-3 fatty acids. In certain embodiments, the omega-3 fatty acids are DHA, EPA, or DPA.

In certain embodiments, the subject is administered less than 400, 600, 800, or 1,000 mg of the lipase or pharmaceutical composition per day. The total units of lipase per meal can be about 10,000, 20,000, 50,000, 100,000, 200,000, 400,000 or more.

In certain embodiments, the lipase or pharmaceutical composition is administered in combination with a fat soluble vitamin (e.g., vitamin A, D, E, or K), an acid blocker, or a nutritional formula containing triglycerides.

In certain embodiments, the subject is a mammal. In certain embodiments, the subject is a human.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1—Lipase Selection of Lipase Engineering

This example describes the design of recombinant mutant Burkholderia cepacia lipases with improved stability against the harsh conditions of the fed-state stomach (low pH and pepsin) and against proteolytic degradation across the length of the gastrointestinal (GI) tract and gastric transit time through the small intestine while maintaining high levels of activity across pH 3.5 to 7 against physiologically relevant fats.

The goals for lipase engineering were to design a lipase enzyme with one or more of the following features:
inherent stability against the harsh conditions of the fed-state stomach (low pH and pepsin) and against proteolytic degradation across the length of GI tract through the small intestine while maintaining high levels of activity across pH 3.0 to 7;
improved stability against proteolysis without loss of activity in relevant pH ranges;
activity and survivability at relevant pH's and against proteolytic degradation across length of GI tract of interest (stomach, duodenum, jejunum, proximal ileum);
maintenance of a high activity profile (units/mg) compared to wild-type lipase; and
stabilized to start digesting fats into absorbable fatty acids and monoglycerides in the stomach.

The benchmark physiological residence times of fat for people with exocrine pancreatic insufficiency (EPI) were used as the basis for the lipase engineering goals. Specifically, for people with EPI (1) transit time through the low pH environment in the stomach is about 60-90 minutes, (2) transit time through stomach proteases (e.g., pepsin) is from about 90 to about 120 minutes, depending upon the content of the meal consumed, and (3) transit time through the small intestine is from about 240 to about 360 minutes.

Lipase characteristics for engineering consideration included:
Lipase activity at physiologically relevant pH conditions of the digestive tract (pH 3.5-7) without the need for enteric coating;
Ability to digest biologically relevant fats including long-chain polyunsaturated triglycerides (such as DHA) and trioleic acid (pure oleic acid triglyceride; major component of olive oil and common triglyceride in the standard human diet);
Lipase solubility at physiologically relevant pH conditions of the digestive tract.
Co-lipases not required for activity;
Not inhibited by bile salts;
Hydrolysis preference for sn-1 and sn-3 positions on the triglyceride over the sn-2.
Survivability at low pH;
Survivability against pepsin in the stomach;
Survivability against proteolytic degradation in particular the A. melleus protease with which the lipase will be co-formulated; and
Thermostability at 37° C. (body temperature).

Numerous lipases were screened and evaluated of activity at physiologically relevant pH conditions (pH 4.0-7.0) with and without bile salts. Exemplary lipases are shown in FIG. 3 and sequence alignments of exemplary lipases are shown in FIGS. 4 and 5.

The base (i.e., starting) lipase used for mutational analysis was derived from B. cepacia, a microbially derived I.2 class of lipase enzyme, and had the amino acid sequence of SEQ ID NO: 1. The I.2 lipase was selected from lipase enzymes that were tested against a wide range of fats (triglycerides) including the most difficult fats to digest such as omega-3 fats (DHA and EPA triglycerides). An I.2 class lipase was chosen for mutational analysis because these lipases exhibit (i) high activity against long-chain poly-unsaturated fatty acids (LCPUFAs) such as DHA, (ii) a broad level of activity at physiologically relevant pH range (pH 3.5-7) and (iii) high activity with and without bile salts.

### Lipase Engineering

In general, protein engineering was used to select and improve the characteristics of a protein using the following iterative process:
1. Identify key attributes of the protein and develop robust high-throughput analytical methods to test each key attribute.
2. Make changes to the protein's amino acid sequence though site directed mutagenesis to produce an array of variants.
3. Express and test each variant against each analytical method and rank how each change affected protein performance.
4. Select the top variants to advance for further testing that meet pre-defined study goals.

At the end of Step 4—the optimal performing variant is used as the “parent” or “base” sequence for the next round and steps 2 through 4 are repeated until the variants produced have the desired characteristics.

Three-dimensional molecular modelling of B. cepacia lipase identified amino acids on the surface of the protein as low pH and proteolytic degradation often occurs on the surface of the protein. Without wishing to be bound by theory, it is believed that low pH and proteolytic degradation on the protein surface can lead to improper folding of the lid or the subdomain, which can reduce activity of the enzyme.

B. cepacia lipase (SEQ ID NO: 1) is a member of the I.2 subfamily of bacterial lipases. Each lipase in this subfamily is structurally related and has a number of common features. In addition, the I.1 and I.2 families of lipase share relatively high amino acid sequence similarities and share a number of structural features including a serine-histidine-aspartate active domain, calcium binding sites, a lid and subdomain which serve to protect the active site, a disulfide bridge, and require a foldase (lipase-specific foldase (‘Lif’)) to ensure correct folding.

Amino acid substitutions considered for mutagenesis were derived from evaluation across numerous I.2 lipases. It is contemplated that improved product characteristics for the B. cepacia lipase should be applicable to other members of the Family I lipases including, subgroups I.1, I.2 and I.3. To illustrate this approach, a portion of the phylogenetic tree of bacterial lipases is presented in FIG. 3, and a sequence alignment of selected I.1, I.2 and I.3 bacterial lipases is presented in FIG. 4.

### Lipase Engineering

A large number of mutant B. cepacia lipases were designed, each with up to three amino acid substitutions relative to the wild-type sequence. In the initial round, each mutant DNA sequence contained up to three amino acid changes (called substitutions) and when expressed, produced a protein with three amino acid changes called a variant.

The top selected distinct amino acid substitutions in B. cepacia lipase are listed in TABLE 4.

TABLE 4
Top Substitutions

| | |
|---|---|
| F221L | G125S |
| D102Q | K165Q |
| N154H | N300Y |
| S153N | S281A |
| V266L | V138I |
| F249L | T79Q |
| A128N | L91M |
| L161A | T137A |

A listing of the top combinations of the amino acids from one of the initial rounds is set forth in TABLE 5.

TABLE 5

| Variant | Amino Acid | Amino Acid | Amino Acid |
|---|---|---|---|
| No. | Change 1 | Change 2 | Change 3 |
| V130 | D102Q | N154H | F221L |
| V101 | T79Q | V266L | L287V |
| V133 | L91M | V220A | V266L |
| V172 | G125D | D159N | F249L |
| V190 | Q39A | T137A | F249L |

Briefly, DNA fragments encoding the mutant B. cepacia lipases were cloned into an expression vector, and all the constructs were confirmed by gene sequencing. The lipase enzyme was expressed into the periplasmic space of Escherichia coli. The outer cell membrane was ruptured using osmotic shock and the lipase was harvested.

The recombinant mutant B. cepacia lipases were tested for lipase activity, pH survivability, pepsin protease stability, and A. melleus protease proteolytic stability as described in Example 2, Example 3, Example 4, and Example 5, respectively.

An analysis of the recombinant mutant B. cepacia lipases from the lipase engineering campaign revealed that variant V130 had the best net positive effects across the conditions tested. V130 contained three substitutions (D102Q, N154H, and F221L). The V130 variant substitutions were further modified in subsequent rounds where additional B. cepacia lipases were designed. An additional 13 substitutions were selected for use given their impact on one or more of enzyme activity, pH survivability, pepsin protease stability, and A. melleus protease proteolytic stability. The substitutions carried forward are shown in TABLE 4, supra.

### Example 2—Lipase Activity Assay

Goals of the lipase engineering campaign were to produce a mutant lipase that is active across a broad fed-state range from pH 4 to pH 7, is not inhibited by bile salts, is soluble in this pH range and is stable at 37° C. This example describes lipase assay for determining whether the lipase mutants are likely to exhibit activity in the portions of the digestive tract where hydrolysis and nutrient absorption takes place.

Lipase activity against LCPUFA-triglycerides was tested because, given their chain length and double bonds, LCPUFAs are very challenging to digest. Additionally, lipase activity was tested against oleic acid, the primary component of olive oil, and a high component in a standard human diet.

In contrast to mammalian lipases, B. cepacia lipase does not require co-lipase for catalytic activity and was demonstrated to be stable in the presence or absence of bile salts. B. cepacia lipase catalyzes the hydrolysis of triglycerides to produce fatty acids and monoglycerides with a greater level of activity against the sn-1 and sn-3 regions of the triglyceride and thereby have functionalities similar to human pancreatic enzyme.

LCPUFAs are triglycerides usually found in fish oil. Preference for hydrolytic activity in sn-1 and sn-3 regions is consistent with human pancreatic lipase which allows for fats to be digested into two fatty acids and one-monoglyceride for absorption into plasma and incorporation into enterocytes and tissues.

The turnover of a given substrate (e.g., fat) is driven by enzyme activity (units/mg that survives degradation) and the interaction of the enzyme to the substrate at relevant pH levels.

To ensure that the lipase is not inhibited by bile salts, the activity assay was also conducted in the presence of 8 mM bile salts at pH 7. This pH was selected as the bile salts are not appreciably soluble at <pH 6. The bile salts used, and their relative ratios, are presented in TABLE 6.

TABLE 6

| Bile Salt | Relative Ratio |
|---|---|
| Cholic acid | 1.7 |
| Deoxycholic acid | 1 |
| Chenodeoxycholic acid | 1.8 |

Fat digestion by lipases occurs at the oil/water interface, and to model this, the lipase activity assay was conducted using a physiologically relevant substrate and creating an oil-water emulsion. To ensure that the assay could differentiate improvement in an engineered lipase, long-chain fats were used as the substrate, because they are more challenging to digest. In addition, LCPUFA deficiencies, in particular DHA and EPA, have been demonstrated in subjects with EPI, CF and/or malabsorption.

For the activity assay, a substrate was selected that is heavily enriched in both DHA triglycerides and in oleic acid triglycerides.

DHA is a triglyceride in which each fatty acid has 22-carbons and contains 6 double-bonds and is one of the longest chain fatty acid commonly encountered in the diet. Furthermore, omega-3 fatty acids such as DHA and EPA are structural components of membranes and also biological mediators involved in the regulation of various physiological functions and so these fatty acids have a critical role in the composition, development and function of heart, liver, and neural tissues, as well as in the regulation of the inflammatory and immunological systems. LCPUFAs and omega-3 fatty acids DHA and EPA in particular, have been shown to be deficient in subjects with EPI, CF and/or malabsorption.

Oleic acid is another fat substrate that is a common dietary fat and makes up about 55% to 83% of olive oil. Given the variety in the composition of olive oil, triolein, a synthetic fat similar olive oil, was selected for use in the activity assay because it provides more consistent results. Each fatty acid of triolein is oleic acid (18-carbons and contains 1 unsaturated bond).

The DHA oil selected for use in the activity assay is algae-derived and contains ˜37% DHA triglycerides and ˜22% oleic acid triglyceride (Nu-chek, Elysian, MN), with the remainder being mostly comprised of myristic acid triglyceride and palmitic acid triglyceride. The amounts of the major components are presented in TABLE 3.

In the assay, the substrate was emulsified in a buffer at the specified pH and the lipase was added to the substrate. After a fixed incubation time (which allows the lipase to digest triglycerides to form soluble free fatty acids), the lipase was heat inactivated. Aliquots were withdrawn and a fatty acid quantitation kit was used that tags the fatty acids with Coenzyme A. Tagged fatty acids were then quantified by either a colorimetric or fluorometric signal. The concentration of each lipase was established using SDS-PAGE and combined with the assay data to provide specific activity.

Given that wild-type B. cepacia lipase has strong activity against DHA across the pH range of interest (pH 4.0 to 7.0) and is not inhibited by bile salts, a goal of the engineering campaign was to ensure that changes made to improve the survivability aspects of the enzyme did not reduce the activity level and did not cause the lipase to become inhibited by bile salts. Accordingly, the goals for lipase engineering focused on the following:
Ensuring that there is high activity in the pH range of 3.0 to 7 so that the enzyme can digest fats from the fed-state stomach through to the end of the jejunum;
Ensuring that the lipase is not inhibited by bile salts at pH 7.0;
Ensuring that the lipase is stable at 37° C.; and
Ensuring that the lipase is soluble in the pH range of interest (pH 3.0 to 7).

Further, each variant produced by the lipase engineering process was tested for activity the following ways:
pH 4 using DHA triglyceride oil substrate at 37° C.;
pH 7 using DHA triglyceride oil substrate at 37° C.; and
pH 7 using DHA triglyceride oil substrate at 37° C. with 8 mM bile salts.

Briefly, DHA oil substrate was emulsified into water and stabilized with gum arabic to form a stable emulsion. The pH was then adjusted by adding the emulsion to an appropriate volume of the specified pH buffer. After 15 minutes, the reaction was stopped by heating to inactivate the lipase. The fatty acids produced were quantified using commercial free fatty acid assay kits (e.g., ABCAM, UK, Free Fatty Acid Assay Kit). The final reaction produced a response which can be detected colorimetrically.

The activity of the substitutions evaluated show a strong correlation between activity at pH 4 and activity at pH 7 indicating that amino acid changes that affect activity at one pH, also affect the activity at other pHs. These substitutions (e.g., S153N, L287V, I232L, Y129N, V143A, A128N, N154H, F249L) in TABLE 1 had the potential to improve activity in the key pH range of interest and were prioritized for further engineering.

### Example 3—pH Survivability Assay

This example describes an assay to determine lipase survivability in the low pH conditions of the stomach.

A goal of the lipase engineering campaign was to produce a lipase capable of surviving the acidic conditions of the digestive system, especially the stomach. The pH of stomach aspirates in children with CF ranges from about 2 to above 5. While the pre-prandial pH is low (˜pH 2), as soon as the meal is consumed, the pH rapidly increases to greater than pH 5, then slowly drops back to pH 2 over about 120 minutes. During the fed-state interval, there is a slow but continuous emptying of the stomach contents through the pyloric valve, and by the time the chyme is below pH 4, more than 60-90% of the meal has transitioned into the duodenum. The wild type (starting) lipase from B. cepacia has good survivability down to pH 4. However, there may be periods of time where the lipase may be subjected to pH levels below pH 4.0. Therefore, one goal of the lipase engineering was to improve survivability down pH 3.0 to 3.5. As the lipase would be taken by patients along with food, survivability at the very low pH of the fasted-state stomach is of less concern. However, there is a risk of inactivation associated with any lipase that remains in the late fed-state stomach when it drops below pH 4.

Accordingly, a lipase having a half-life at pH 3.0-3.5 of at least 60 to 90 minutes is desirable. This ensures that no more than half of the lipase is inactivated by low acid during passage though the stomach. As the wild-type B. cepacia lipase has a 40 to 50 minute half-life, the objective was to achieve an improvement of 50-100%.

To test the survivability of each lipase produced by lipase engineering, a high-throughput microtiter plate assay was developed to assess the lipase survivability at low pH and 37° C. In this pH range (pH 3.2-3.5), the wild-type lipase has a half-life of 40 to 50 minutes and the method is sufficiently discriminating that it is possible to differentiate the effects of amino acid substitutions and relevant improvements in lipase survivability.

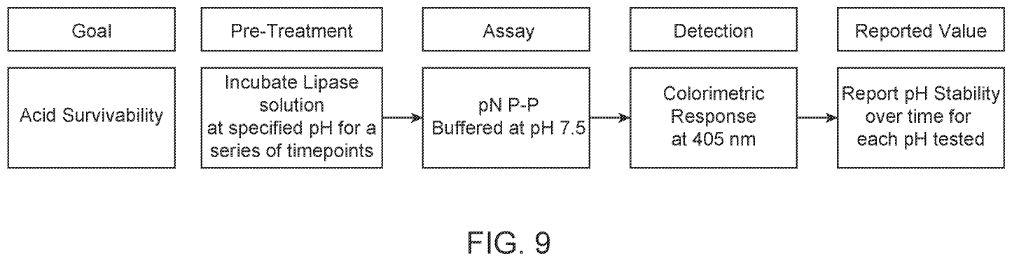

A flow chart describing the acid survivability assay is shown in FIG. 9. Briefly, the lipase was added to a buffer at the assay pH (3.0 to pH 3.3) at 37° C. for 30 minutes to 120 minutes. At each time interval, an aliquot was withdrawn, and the pH was neutralized. The activity of each aliquot was measured using a synthetic substate, p-nitrophenyl palmitate (p-NPP) which is cleaved by lipases to form p-nitrophenol which is quantified by either a colorimetric or fluorometric signal. The data was then compared to a control which was not exposed to acid, and the data was analyzed to establish the half-life. A description of the method is provided in part (a) below.

As lipase engineering progressed, the variants were expected to have improved survival. If, within the timeframe of the method, the improvements made it difficult to discriminate among variants, the pH was lowered to increase the stringency and assist in differentiating the variants. Accordingly, the top variants were tested for pH survivability the following way, to allow for differentiation among the variants: pH ˜3.0-3.5 or below using p-NPP substrate at 37° C. for 2 hours.

pH Survivability Assay Using p-NPP Fluorometric Detection

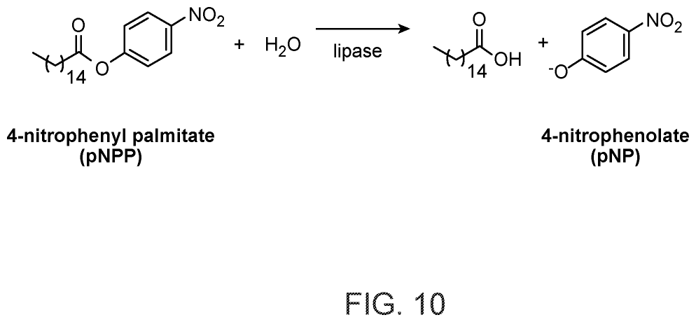

In each well of a 96-well plate, a sample of each lipase-containing periplasm was added simultaneously to 2× concentrated buffer set at the specified assay pH. The addition time was considered to be T=0 for the survivability assay. At specified timepoints, an aliquot was withdrawn and transferred to a daughter plate. The reaction volume was diluted 1:9 for a 1/10th dilution into the stop/indicator buffer. The pH shift arrested any acid-mediated degradation. The surviving lipase started to hydrolyze the p-NPP colorimetric substrate. The reaction of p-NPP (p-nitrophenyl palmitate) with lipase produced palmitic acid and para-Nitrophenolate which has a strong colorimetric response at 405 nm and can be measured. The mechanism of p-NPP hydrolysis by lipase reaction is depicted in FIG. 10. The daughter plate was read continuously in kinetic mode at 405 nm. Each successive timepoint in the experiment had less surviving lipase and the kinetic curve had a shallower slope. The slopes of each timepoint were used to establish the half-life of each lipase variant. The variants were run alongside assay controls which included a WT-lipase control (expressed in E. coli) as well as a control of commercially purchased purified WT lipase (Amano Enzyme, Nagoya, Japan).

From the standpoint of survivability, it was desirable that improvements to pH survivability not adversely affect performance against protease and vice versa.

### Example 4—Pepsin Survivability Assay

This example describes an assay to determine lipase proteolytic survivability in pepsin conditions.

In certain embodiments, the engineered lipase survives the pepsin that is present in the stomach. This may not be an issue for the standard of care products based on pancrelipase as they have enteric coatings that prevent the enzymes from being exposed to pepsin in the stomach. In contrast, the engineered B. cepacia lipases described herein, in certain embodiments, are designed to be immediately available in the stomach and will be exposed to pepsin. Pepsin is an aspartic acid protease with maximum activity at low pH levels (pH 1.5 to 4). As such, the engineered B. cepacia lipases were evaluated for the impact of pepsin on lipase survivability.

To test the survivability of each lipase variant, a high-throughput microtiter plate assay was developed to assess the lipase survivability with pepsin at ˜pH 4 and 37° C. The initial amount of pepsin added was based upon the USP chapter for Simulated Gastric Fluid (SGF) Test Solution which suggests a pepsin concentration of 3.2 mg/mL. Pepsin was added to the lipase solution to form a solution that was 3.2 mg/mL with respect to pepsin and 0.01 mg/mL with respect to lipase. As the lipase engineering progressed, the amount of pepsin was increased to force differentiation without prolonging the assay time. Preliminary engineering used 19 mg/mL (6× over USP) and later used 32 mg/mL (10× over USP). This condition may be harsher than the normal conditions of the stomach because there is no background protein present and as such, there is no protein for the pepsin to attack other than the lipase. Accordingly, the method was capable of distinguishing the effect of amino acid substitutions on survivability of the lipase variants.

In the assay, the lipase was added to a buffer at pH 4 containing pepsin. At each time interval, an aliquot was withdrawn, and the pH was neutralized to inactivate the pepsin. The activity of each aliquot was measured using a synthetic substate, p-nitrophenyl palmitate (p-NPP) which was cleaved by lipases to form p-nitrophenol which was quantified by either a colorimetric or fluorometric signal. The data was then compared to a control which was not exposed to pepsin and the data analyzed to establish the lipase survivability expressed as half-life.

The goal for the engineered lipase was to ensure that the lipase at 0.01 mg/mL has a half-life at pH 4 with 32 mg/mL of pepsin of at least 90-120 minutes. Given the excessive amount of pepsin present, this goal ensured that minimal lipase was not inactivated by pepsin during passage though the stomach. As the wild-type B. cepacia lipase has a 50 to 70 minute half-life, a minimum of a 50-125% improvement was desirable.

Each variant produced during the lipase engineering process was tested for pH survivability by adding 0.01 mg/mL lipase to 32 mg/mL pepsin at pH 3.5. Detection using p-NPP substrate, as described in detail below, was performed at 37° C. for 30 minutes.

The top variants were tested for pH survivability by adding 0.01 mg/mL lipase to 32 mg/mL pepsin at pH 3.5. Detection using p-NPP substrate, as described in detail below, was performed at 37° C. for 2 hours.

As lipase engineering progressed, the variants were expected to have improved survival. If, within the time frame of the method, the improvements made it difficult to discriminate among variants, the pH was lowered to increase the action of the pepsin and assist in differentiating the variants.

Pepsin Survivability Assay Using p-NPP Fluorometric Detection

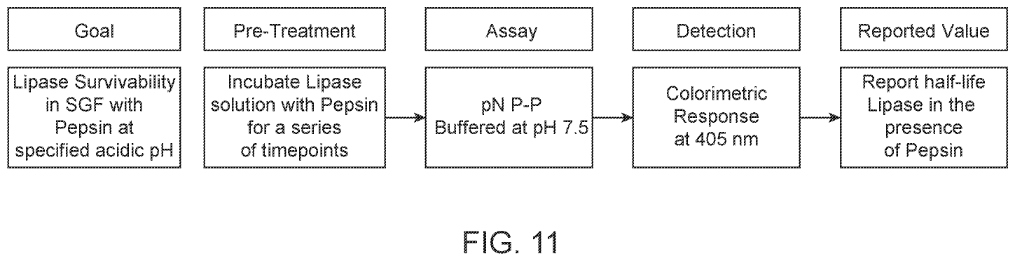

A flow chart showing the pepsin survivability assay is shown in FIG. 11.

In each well of a 96-well plate, a sample of each lipase-containing periplasm was added simultaneously to 2× concentrated buffer set at the specified assay pH containing pepsin at the specified concentration. At specified timepoints, an aliquot was withdrawn and transferred to a daughter plate. The reaction volume was diluted 1:9 for a 1/10th dilution into the stop/indicator buffer. The surviving lipase started to hydrolyze the p-NPP colorimetric substrate. The reaction of p-NPP (p-nitrophenyl palmitate) with lipase produced palmitic acid and para-Nitrophenolate which has a strong colorimetric response at 405 nm. This reaction is depicted in FIG. 10. Each successive timepoint in the experiment had less surviving lipase and the kinetic curve had a shallower slope. The slopes of each timepoint were used to establish the half-life of each lipase variant. The engineered lipase variants were run alongside assay controls which included a wild type-lipase control (expressed in E. coli) as well as a control of purified wild type lipase (Amano Enzyme, Nagoya, Japan).

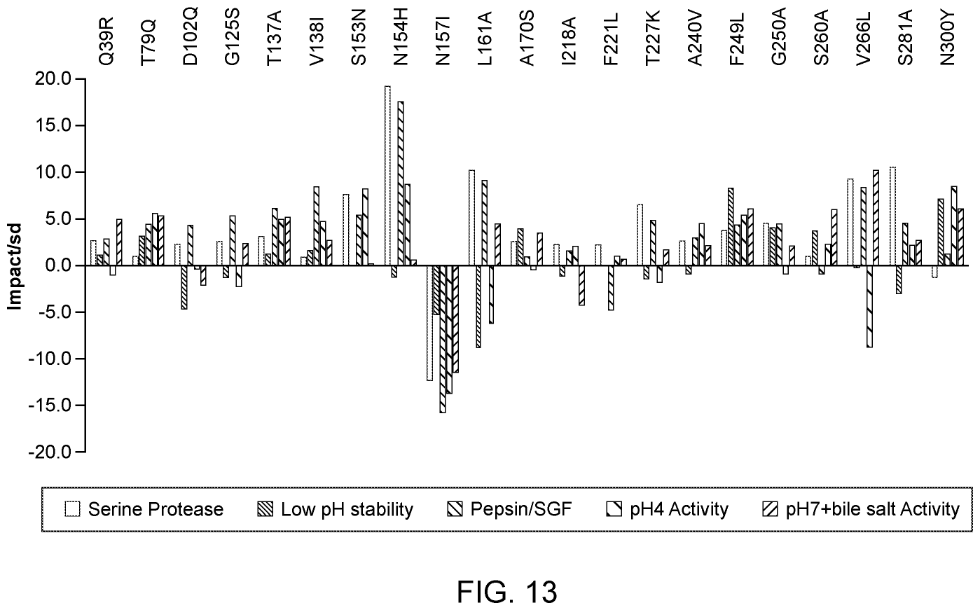

Exemplary results for pepsin stability for various mutations and variants are shown, e.g., in FIGS. 13-16 and 19.

### Example 5—Proteolytic (A. melleus Protease) Survivability Assay

This example describes an assay to determine lipase survivability under A. melleus protease conditions.

A goal for lipase engineering was improved survivability of the lipase in the presence of proteases present in the stomach and the small intestine. An engineered lipase may be delivered in combination with a protease and an amylase for protein and starch digestion, respectively. As such, the lipase may be exposed to the protease from A. melleus for co-dosing. A. melleus protease is a serine protease with a maximum activity at pH 7 to pH 8 and a pH range of more than 50% activity from pH 5 to pH 11. Unlike mammalian proteases such as trypsin and chymotrypsin, which cleave proteins only after specific amino acids, the A. melleus protease (also called SAP or oryzin) cleaves proteins non-specifically down to small oligomers and individual amino acids. As such, the A. melleus protease provides a representative harsh condition to evaluate the engineered lipase survivability against pancreatic proteases. If selected for use in combination, the engineered lipases are expected to be in the presence of the A. melleus protease for three to six hours (the transit time from the fed state stomach through the small intestine), so it is desirable that the engineered lipase is resistant to degradation by this protease.

To test the survivability of each lipase produced by lipase engineering, a high-throughput microtiter plate assay was developed to assess the lipase survivability with A. melleus protease at pH 6 to pH 7 and 37° C. This pH was selected as it is in the range of maximum A. melleus protease proteolytic activity and represents atypical pH found in the small intestine. Initial studies were performed with 3.3 mg/mL protease together with 0.01 mg lipase at pH 6.0. This amount of protease added was selected to allow for differentiation with the experiment timeframe requirements of under an hour. For this in vitro assay, the ratio of protease to lipase was at least 100-fold higher than in the anticipated formulation for co-administration. This condition may be harsher than the normal conditions of the stomach because there is no background protein present and as such, there is no protein for the protease to attack other than the lipase. Accordingly, the method was capable of distinguishing the effect of amino acid substitutions on survivability of the lipase variants.

In the assay, described in more detail below (see, “A. melleus Protease Survivability Assay Using p-NPP Fluorometric Detection”), the lipase was added to a buffer at pH 6.0 containing the protease. At each time interval, an aliquot was withdrawn, and the pH was neutralized to inactivate the protease. The activity of each aliquot was measured using a synthetic substrate, p-nitrophenyl palmitate (p-NPP) which was cleaved by lipases to form p-nitrophenol which was quantified by either a colorimetric or fluorometric signal. The data was then compared to a control which was not exposed to protease and the data analyzed to establish the lipase half-life.

Conditions in follow-on experiments as part of the final analysis were set to model a more realistic case, that of a fed-state intestine for a normal (non-pancreatic insufficient) test subject. In these cases, the ratio was 0.33 mg/mL protease together with 0.01 mg lipase and 10 mg/mL casein at pH 6.0.

Initial experiments under these conditions showed improvement in survivability.

A. melleus Protease Survivability Assay Using p-NPP Fluorometric Detection

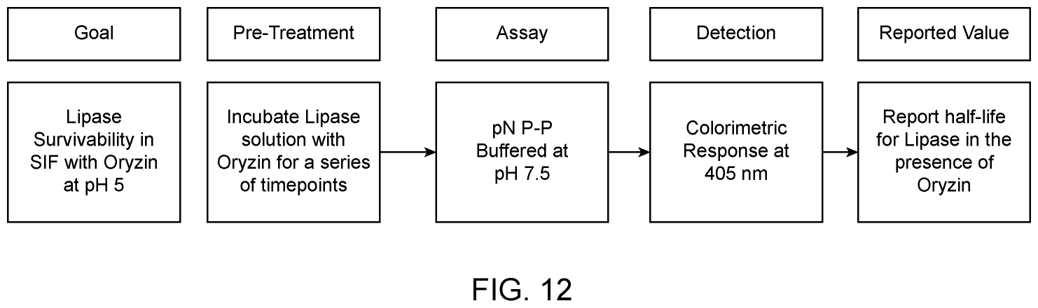

An overview of the A. melleus survivability assay is depicted in FIG. 12.

In each well of a 96-well plate, a sample of each lipase-containing periplasm was added simultaneously to 2× concentrated buffer set at the specified assay pH (centered on pH 6) containing A. melleus protease at the specified concentration. The final concentration of A. melleus protease ranged from 1.6 mg/mL to 3.3 mg/mL. The addition time was considered to be T=0 for the survivability assay. At specified timepoints, an aliquot was withdrawn and transferred to a daughter plate and diluted directly into indicator buffer as described for the p-NPP assays above. The surviving lipase started to hydrolyze the p-NPP colorimetric substrate. The reaction of p-NPP (p-nitrophenyl palmitate) with lipase produces palmitic acid and para-Nitrophenolate which has a strong colorimetric response at 405 nm. This reaction is depicted in FIG. 11. The daughter plate was read continuously in kinetic mode at 405 nm. As the oryzin was still active, the kinetic curves bent over with time (as more lipase was inactivated through continuing proteolysis). Each successive timepoint in the experiment had less surviving lipase and the kinetic curve had a shallower initial slope. The slopes of each timepoint were used to establish the half-life of each lipase variant. The engineered lipase variants were run alongside assay controls which include a WT-lipase control (expressed in E. coli) as well as a control of commercially purchased purified WT lipase (Amano Enzyme, Nagoya, Japan).

The data for each mutant was analyzed to determine the impact of each individual substitution of survivability expressed as half-life. The data was well-correlated with the model predictions (p-value of less than 0.01), indicating that the model is highly predictive of the observed survival half-lives.

### Example 6—Further Lipase Engineering

This example illustrates further steps in the B. cepacia lipase engineering process.

Information relating to the substitutions that improved each desired characteristic and which substitutions were detrimental to each desired characteristic were considered when modeling to predict new amino acid substitutions. Modeling proposed new amino acid substitutions to be evaluated further.

The top mutant B. cepacia lipases are indicated as variants in TABLE 7

TABLE 7

| Variant | Amino Acid | Amino Acid | Amino Acid | Amino Acid | Amino Acid | Amino Acid |
|---|---|---|---|---|---|---|
| Code | Change 1 | Change 2 | Change 3 | Change 4 | Change 5 | Change 6 |
| V290 | D102Q | G125S | N154H | F221L | V266L | N300Y |
| V235 | D102Q | T137A | F221L | E35S | G250A | V305I |
| V259 | D102Q | N154H | L161A | F221L | S281A | I218A |
| V272 | L91M | D102Q | A128N | N154H | F221L | Q177A |
| V282 | D102Q | S153N | N154H | F221L | Q39R | T92S |

The variants contained between 2 and 6 additional amino acid substitutions on top of those included in the V130 base variant. Each variant lipase was expressed and evaluated against each test in the analytical battery (stability at low pH, stability in the presence of proteases, etc.). Each new substitution was present in 5 different variants across the array, which provided sufficient repetition to allow multivariable statistical deconvolution tools to be used to identify which amino acid substitutions were responsible for improvements. Unless otherwise indicated, the mutational design, expression, purification, and pH, pepsin, and A. melleus protease stability assays were all conducted as described above.

The survivability in the presence of low acid conditions pH 3.2 and in the presence of A. melleus protease were tested in the same manner as previously performed.

Although wild-type B. cepacia lipase demonstrated some resistance to pepsin, one goal for engineering was to improve survivability against pepsin to 90 to 120 minutes. Initially, at 19 mg/mL of pepsin and pH 4.0, the variants could not be differentiated. After some engineering, to increase the stringency, the concentration of pepsin was increased to 32 mg/mL and the pH of the challenge was lowered to 3.8 to increase the action of the pepsin and assist in differentiating beneficial amino acid substitutions.

The top substitutions, based on a combination of desired characteristics (low pH survival, survival against aspartic proteases, and serine proteases) were used.

An analysis of the recombinant mutant B. cepacia lipases of the lipase engineering campaign identified variant V290 that contained six substitutions (D102Q, N154H, F221L, V266L, G125S and N300Y). The V290 variant substitutions were moved forward to form the base sequence—as this variant had the best net positive effects and the six substitutions were known to work well together. As a result, a mutant B. cepacia lipase enzyme containing these six substitutions was used as a parent in the design of additional B. cepacia lipases. Furthermore, 12 additional substitutions were selected for inclusion based upon the strength of improvement shown. The substitutions were based upon the strength of improvement for all parameters of interest and are presented in TABLE 8.

TABLE 8

| No. | Substitution |
|---|---|
| 1 | F221L |
| 2 | G125S |
| 3 | N154H |
| 4 | N300Y |
| 5 | V266L |
| 6 | D102Q |
| 7 | F249L |
| 8 | S153N |
| 9 | T137A |
| 10 | T79Q |
| 11 | G250A |
| 12 | S281A |
| 13 | T227K |
| 14 | A170S |
| 15 | A240V |
| 16 | Q39R |
| 17 | S260A |
| 18 | V138I |

TABLE 9 illustrates the top B. cepacia lipase amino acid substitutions for pH survivability.

TABLE 9

| No. | Substitution |
|---|---|
| 1 | F249L |
| 2 | N300Y |
| 3 | G250A |
| 4 | A170S |
| 5 | S260A |
| 6 | T79Q |
| 7 | V138I |
| 8 | T137A |
| 9 | Q39R |
| 10 | F221L |

TABLE 10 illustrates the top B. cepacia lipase amino acid substitutions for serine protease survivability.

TABLE 10

| No. | Substitution |
|---|---|
| 1 | N154H |
| 2 | S281A |
| 3 | L161A |
| 4 | V266L |
| 5 | S153N |
| 6 | T227K |
| 7 | G250A |
| 8 | F249L |
| 9 | T137A |
| 10 | Q39R |

TABLE 11 illustrates the top B. cepacia lipase amino acid substitutions for pepsin survivability.

TABLE 11

| No. | Substitution |
|---|---|
| 1 | N154H |
| 2 | L161A |
| 3 | V138I |
| 4 | V266L |
| 5 | T137A |
| 6 | S153N |
| 7 | G125S |
| 8 | T227K |
| 9 | S281A |
| 10 | G250A |

Exemplary data for certain amino acid substitutions evaluated for inclusion are shown FIG. 13. Stability in the presence of A. melleus protease, stability at low pH, stability in the presence of pepsin/SGF, activity at pH 4, and activity at pH 7 in the presence of bile salts were evaluated for each amino acid substitution using multivariable statistical deconvolution tools as described above. As shown, certain mutations resulted in positive changes in stability under certain conditions but not under others. Some mutations resulted in increased stability but decreased activity (see, e.g., V266L). In addition, some mutations resulted in a decrease in all variables tested (see, e.g., N1574), and in most cases such mutations were not advanced through the selection process. However, various amino acids were ultimately selected for further testing in combination.

### Example 7—Final Lipase Engineering

This example illustrates B. cepacia lipase engineering using the best variants identified above.

Unlike in earlier rounds, where new substitutions were introduced, the purpose of the final rounds was to recombine all the best performing amino acid substitutions from the previous rounds in different configurations to achieve additive, synergistic, or potentiating improvements of the product characteristics. In the final round, 46 variants and were denotated V301-V346. Each final variant contained between 8 and 11 amino acid substitutions (˜3% change as compared to the starting, wild-type B. cepacia lipase sequence). The full listing for the final variants is shown in TABLE 12.

TABLE 12

| Variant | Amino Acid Change | | | | | | | | | | |
|---|---|---|---|---|---|---|---|---|---|---|---|
| Code | 1 | 2 | 3 | 4 | 5 | 6 | 7 | 8 | 9 | 10 | 11 |
| V301 | T79Q | D102Q | G125S | T137A | S153N | N154H | F221L | V266L | N300Y | | |
| V302 | T79Q | D102Q | G125S | T137A | N154H | F221L | F249L | V266L | N300Y | | |
| V303 | T79Q | D102Q | G125S | S153N | N154H | F221L | F249L | V266L | N300Y | | |
| V304 | D102Q | G125S | T137A | S153N | N154H | F221L | F249L | V266L | N300Y | | |
| V305 | T79Q | D102Q | G125S | T137A | S153N | N154H | F221L | V266L | N300Y | T227K | |
| V306 | T79Q | D102Q | G125S | T137A | S153N | N154H | F221L | F249L | V266L | N300Y | |
| V307 | T79Q | D102Q | G125S | T137A | S153N | N154H | F221L | V266L | N300Y | G250A | |
| V308 | T79Q | D102Q | G125S | T137A | S153N | N154H | F221L | V266L | S281A | N300Y | |
| V309 | T79Q | D102Q | G125S | T137A | N154H | F221L | F249L | V266L | N300Y | T227K | |
| V310 | T79Q | D102Q | G125S | T137A | N154H | F221L | V266L | N300Y | T227K | G250A | |
| V311 | T79Q | D102Q | G125S | T137A | N154H | F221L | V266L | S281A | N300Y | T227K | |
| V312 | T79Q | D102Q | G125S | T137A | N154H | F221L | F249L | V266L | N300Y | G250A | |
| V313 | T79Q | D102Q | G125S | T137A | N154H | F221L | F249L | V266L | S281A | N300Y | |
| V314 | T79Q | D102Q | G125S | T137A | N154H | F221L | V266L | S281A | N300Y | G250A | |
| V315 | T79Q | D102Q | G125S | S153N | N154H | F221L | F249L | V266L | N300Y | T227K | |
| V316 | T79Q | D102Q | G125S | S153N | N154H | F221L | V266L | N300Y | T227K | G250A | |
| V317 | T79Q | D102Q | G125S | S153N | N154H | F221L | V266L | S281A | N300Y | T227K | |
| V318 | T79Q | D102Q | G125S | S153N | N154H | F221L | F249L | V266L | N300Y | G250A | |
| V319 | T79Q | D102Q | G125S | S153N | N154H | F221L | F249L | V266L | S281A | N300Y | |
| V320 | T79Q | D102Q | G125S | S153N | N154H | F221L | V266L | S281A | N300Y | G250A | |
| V321 | T79Q | D102Q | G125S | N154H | F221L | F249L | V266L | N300Y | T227K | G250A | |
| V322 | T79Q | D102Q | G125S | N154H | F221L | F249L | V266L | S281A | N300Y | T227K | |
| V323 | T79Q | D102Q | G125S | N154H | F221L | V266L | S281A | N300Y | T227K | G250A | |
| V324 | T79Q | D102Q | G125S | N154H | F221L | F249L | V266L | S281A | N300Y | G250A | |
| V325 | D102Q | G125S | T137A | S153N | N154H | F221L | F249L | V266L | N300Y | T227K | |
| V326 | D102Q | G125S | T137A | S153N | N154H | F221L | V266L | N300Y | T227K | G250A | |
| V327 | D102Q | G125S | T137A | S153N | N154H | F221L | V266L | S281A | N300Y | T227K | |
| V328 | D102Q | G125S | T137A | S153N | N154H | F221L | F249L | V266L | N300Y | G250A | |
| V329 | D102Q | G125S | T137A | S153N | N154H | F221L | F249L | V266L | S281A | N300Y | |
| V330 | D102Q | G125S | T137A | S153N | N154H | F221L | V266L | S281A | N300Y | G250A | |
| V331 | D102Q | G125S | T137A | N154H | F221L | F249L | V266L | N300Y | T227K | G250A | |
| V332 | D102Q | G125S | T137A | N154H | F221L | F249L | V266L | S281A | N300Y | T227K | |
| V333 | D102Q | G125S | T137A | N154H | F221L | V266L | S281A | N300Y | T227K | G250A | |
| V334 | D102Q | G125S | T137A | N154H | F221L | F249L | V266L | S281A | N300Y | G250A | |
| V335 | D102Q | G125S | S153N | N154H | F221L | F249L | V266L | N300Y | T227K | G250A | |
| V336 | D102Q | G125S | S153N | N154H | F221L | F249L | V266L | S281A | N300Y | T227K | |
| V337 | D102Q | G125S | S153N | N154H | F221L | V266L | S281A | N300Y | T227K | G250A | |
| V338 | D102Q | G125S | S153N | N154H | F221L | F249L | V266L | S281A | N300Y | G250A | |
| V339 | D102Q | G125S | N154H | F221L | F249L | V266L | S281A | N300Y | T227K | G250A | |
| V340 | D102Q | G125S | T137A | S153N | N154H | F221L | F249L | V266L | N300Y | A170S | |
| V341 | D102Q | G125S | T137A | V138I | S153N | N154H | F221L | F249L | V266L | N300Y | |
| V342 | D102Q | G125S | T137A | S153N | N154H | F221L | F249L | V266L | N300Y | Q39R | |
| V343 | G125S | T137A | S153N | N154H | F221L | F249L | V266L | N300Y | | | |
| V344 | D102Q | G125S | T137A | S153N | N154H | F221L | F249L | V266L | N300Y | S260A | |
| V345 | D102Q | G125S | T137A | S153N | N154H | F221L | F249L | V266L | N300Y | A240V | |
| V346 | T79Q | D102Q | G125S | T137A | S153N | N154H | F221L | F249L | V266L | N300Y | G250A |

Unless otherwise indicated, mutational design, expression, purification, and pH, pepsin, and A. melleus protease survivability assays were all conducted essentially as described above. However, a goal of the study was to evaluate and select the best final variants for further analysis.

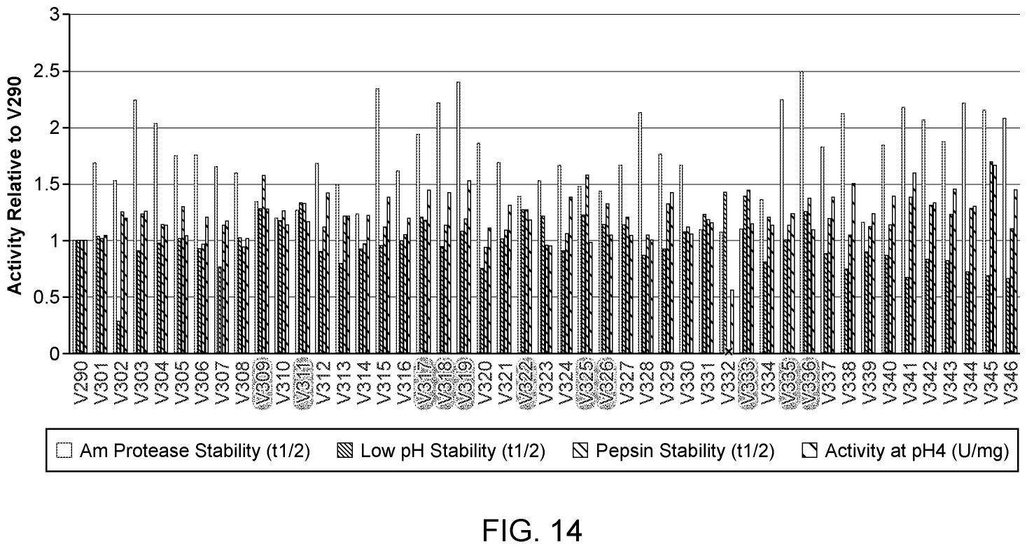

The top performing variants were selected based on their overall ability to show improved survivability against A. melleus protease, pepsin and low pH (pH 3.0) while also maintaining or improving activity at pH 4.0, pH 7.0 and pH 7.0 with bile salts. In the initial rounds of testing, all 46 variants were screened and the results used to narrow the pool to the top 11 variants based upon performance against each of the major characteristics. These data are shown in FIG. 14, with the top 11 variants highlighted.

The top variants were then rescreened to confirm the initial results and allow for more statistical power. In addition to the top 11 variants, the following three controls were assessed: (1) wild-type B. cepacia lipase expressed in E. coli, (2) V130, one of the top variants from initial rounds, (3) V290, one of the top variants from latter rounds. The top variants contained 10 total amino acid substitutions relative to wild type B. cepacia lipase with 320 amino acids, accounting for 97% homology with the wild type B. cepacia lipase. These controls allowed for indexing the performance of each variant against the output of earlier variants in the lipase engineering process to facilitate visualization of the improvement and understand the relative improvements seen.

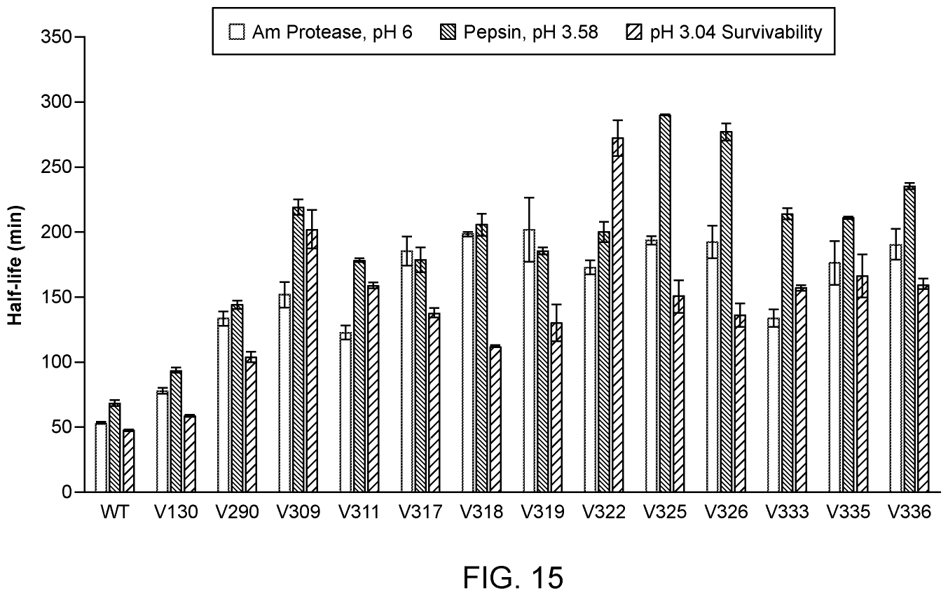

The 11 variants showed excellent survival in both low acid conditions (pH 3.2) and against pepsin (pH 3.8). As such, the pH of the acid challenge was lowered to 3.04 to increase the action of the acid and the pH of the pepsin challenge was lowered to 3.58 to assist in differentiating the variants. A summary of the corresponding survivability data is shown in FIG. 15. The results show that there is a clear progression of improvement from the wild-type lipase through the performance of V130 to V290 to each of the top 11 variants, and with the sole exception of V311 against A. melleus protease, all of the half-lives were higher than V290 for all 11 variants under all conditions tested. The improvement in survivability of the variant lipases at low pH is corroborated by the increases in activity at pH 3.

### Example 8—Selection of the Top Engineered B. cepacia Lipases

This example illustrates the selection of the top two B. cepacia lipase variants using a two-axis approach for the selection, where the top two variants from the standpoint of survivability were advanced as was the best variant from the standpoint of activity.

The data set used to select the top two variants from the standpoint of survivability is presented in TABLE 13.

TABLE 13

| | A. melleus | pH | Pepsin |
|---|---|---|---|
| Name | protease pH 6 | 3.04 | pH 3.58 |
| WT | 53 | 48 | 68 |
| V130 | 78 | 59 | 94 |
| V290 | 134 | 104 | 144 |
| V336 | 191 | 160 | 235 |
| V325 | 194 | 151 | 290 |
| V326 | 193 | 136 | 277 |
| V322 | 173 | 272 | 200 |
| V319 | 202 | 130 | 185 |
| V335 | 176 | 166 | 211 |
| V309 | 152 | 203 | 219 |
| V318 | 199 | 112 | 206 |
| V317 | 186 | 138 | 179 |
| V333 | 134 | 157 | 214 |
| V311 | 123 | 159 | 179 |

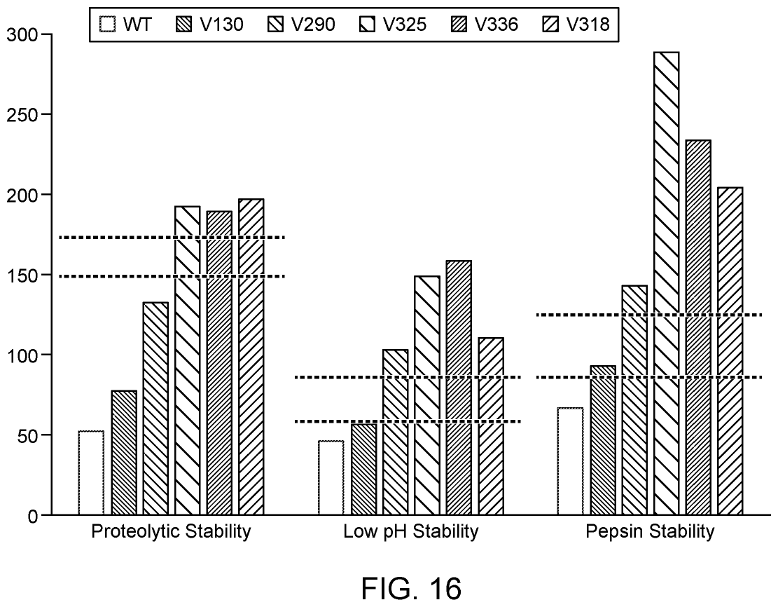

For each of the three survivability characteristics, there was a progression of improvement from the wild-type to the top variant from the initial round (V130) and finally to the top candidates from the final round (V325 and V336). The half-life goal of greater than 150 to 180 minutes for A. melleus protease survival at pH 6 was achieved by the last round of modifications. The final variants survived for more than 190 minutes at pH 6. The half-life goal of 60 to 90 minute survival at pH 3.5 was also achieved. The final variants survived for more than 150 minutes at a more stringent pH of 3.04. The half-life goal of 90 to 120 minute pepsin survival at pH 4 was also achieved. The final variants survived for more than 235 minutes at a more stringent pH of 3.58. Exemplary data and the goals (dotted lines) are provided in FIG. 16.

The improvement factor for each characteristic is listed in TABLE 14, which depicts survivability improvement factors for the four B. cepacia lipase variants compared to the wild type (WT) enzyme.

TABLE 14

| | Oryzin | Low pH | Pepsin |
|---|---|---|---|
| Variant | Improvement | Improvement | Improvement |
| WT | 1.0 | 1.0 | 1.0 |
| V130 | 1.5 | 1.2 | 1.4 |
| V290 | 2.5 | 2.2 | 2.1 |
| V336 | 3.6 | 3.3 | 3.4 |
| V325 | 3.6 | 3.2 | 4.3 |

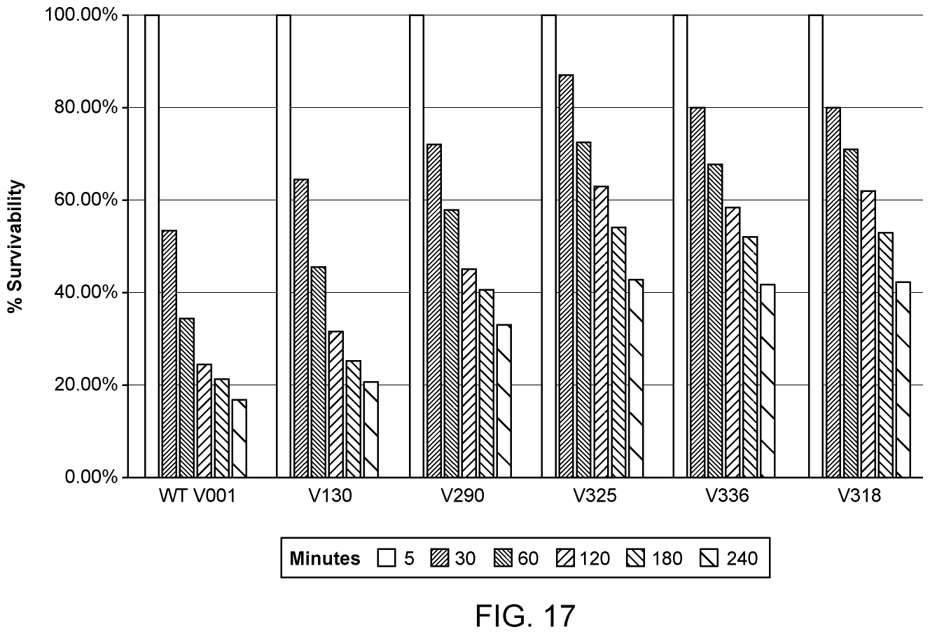

The A. melleus protease improvement factor in the top variants was 3.6-fold more resistant than wild-type. The low pH improvement factor at pH 3.04, in the top variants was 3.2 to 3.3-fold more resistant than wild-type. The pepsin improvement factor at pH 3.58 in the top variants was 3.4 to 4.3-fold more resistant than wild-type. For each survivability test, the percentage of lipase that survives at a series of timepoints is depicted in the charts below. A. melleus protease survivability at pH 6 is shown in FIG. 17 and in TABLE 15, where the top variants (V325 and V336) were compared to the wild-type lipase as well as the top variant (V130) from the initial round and the top variant (V290) from one of the subsequent rounds.

TABLE 15

| Variant | 60 min | 120 min | 180 min | 240 min |
|---|---|---|---|---|
| WT | 34.43% | 24.29% | 21.22% | 16.83% |
| V130 | 45.87% | 31.78% | 25.33% | 20.78% |
| V290 | 57.28% | 45.47% | 40.22% | 32.92% |
| V325 | 72.36% | 62.97% | 53.80% | 42.31% |
| V336 | 67.43% | 58.47% | 51.96% | 41.65% |

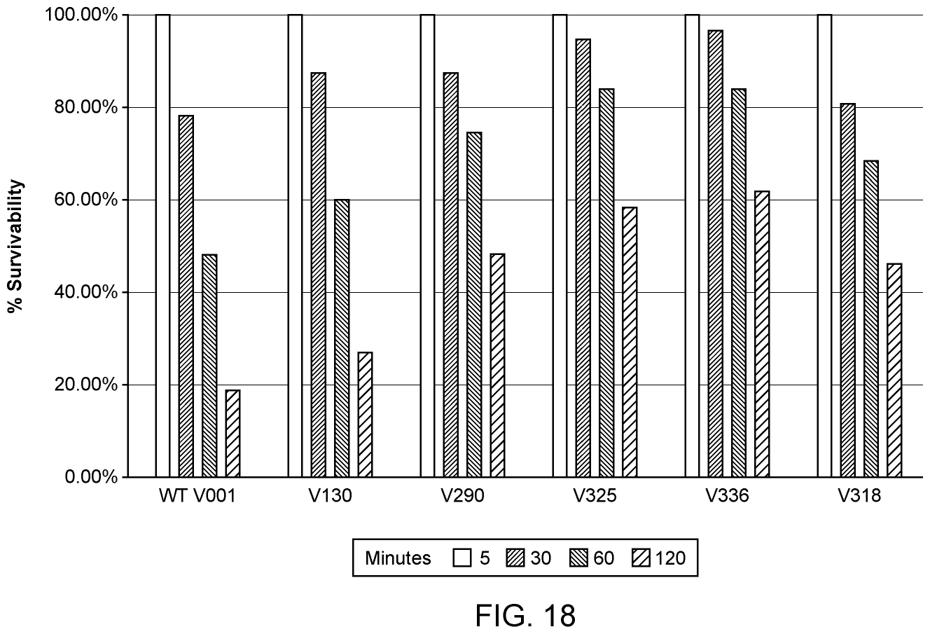

Low pH survivability ay pH 3.0 is shown in FIG. 18 and in TABLE 16.

TABLE 16 Depicts the percentage of lipase that survives at pH 3.0 at a series of timepoints, where the top variants (V325 and V336) were compared to the wild-type lipase as well as the top variant (V130) and the top variant (V290).

TABLE 16

| Variant | 30 min | 60 min | 120 min |
|---|---|---|---|
| WT | 78.26% | 47.81% | 18.58% |
| V130 | 88.00% | 60.31% | 26.88% |
| V290 | 87.94% | 74.20% | 48.62% |
| V325 | 94.66% | 84.02% | 58.11% |
| V336 | 97.07% | 84.23% | 61.93% |

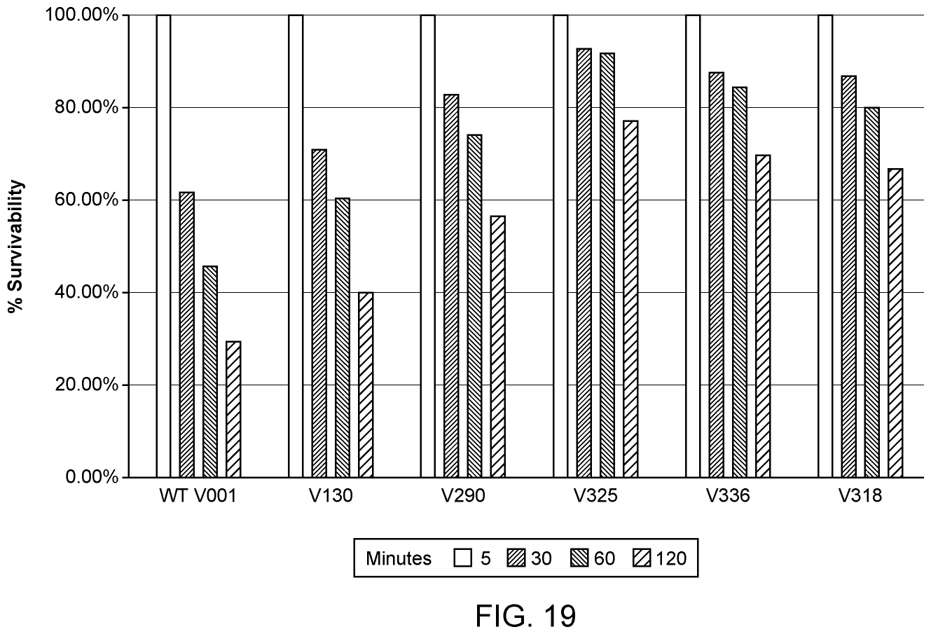

Pepsin survivability at pH 3.58 is shown in FIG. 19 and TABLE 17.

TABLE 17 Depicts the percentage of lipase that survives in the presence of pepsin at pH 3.58 at a series of timepoints, where the top variants (V325 and V336) were compared to the wild-type lipase as well as the top variant (V130) and the top variant (V290).

TABLE 17

| Variant | 30 min | 60 min | 120 min |
|---|---|---|---|
| WT | 61.83% | 45.62% | 29.12% |
| V130 | 70.97% | 60.11% | 40.26% |
| V290 | 82.77% | 73.97% | 56.41% |
| V325 | 92.68% | 92.25% | 77.08% |
| V336 | 87.72% | 84.53% | 69.52% |

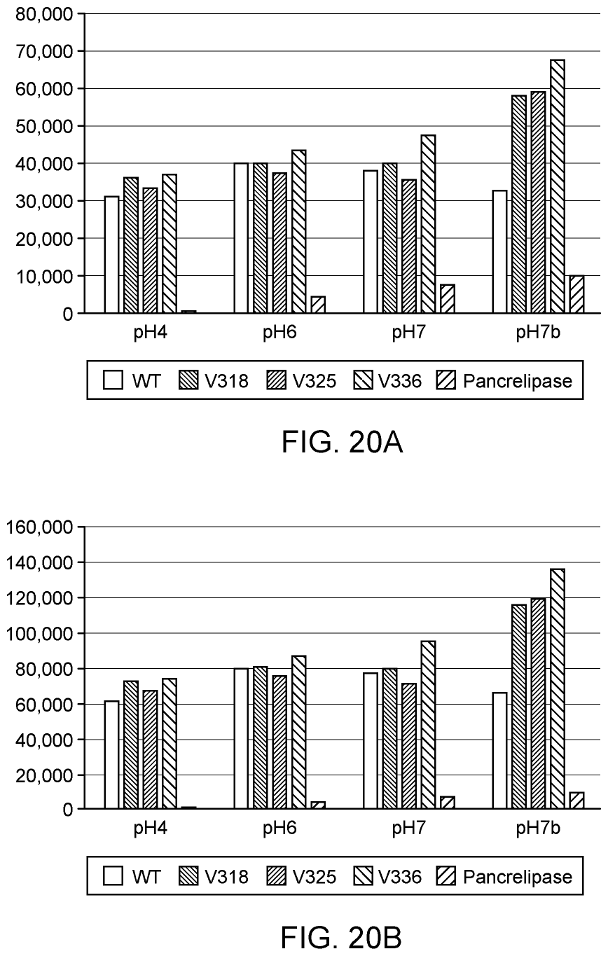

Two of the top variants V325 and V336 were tested head-to-head on the same assay plate against the wild-type lipase as well as against three different concentrations of pancrelipase. The resulting data are presented in FIG. 20A and FIG. 20B. In this chart, the activity of 40 mg and 80 mg of each lipase variant are presented alongside the activity of four capsules of pancrelipase (4×300 mg-1,200 mg pancrelipase in total).

In the key pH range of 4 to 7, each of the top variants (V325 and V336), have specific activities that are either comparable to or show modest improvements over the wild-type specific activity. This ensures that each of these candidates can digest fats from the fed-state stomach through to the end of the jejunum and proximal ileum. None of the top variants (V325 and V336), were inhibited by bile salts. In the key pH range of 4 to 7, and on a per-meal basis, 80 mg of the wild-type lipase or all of the variants had a specific activity at least 10-fold higher than that of 4 capsules of pancrelipase. At pH 4, the pancrelipase had almost no activity. Given that the literature reports that that porcine pancreatic preparations such as pancrelipase are degraded by acid, this result was not surprising because pancrelipase needs a pH 5.5 enteric coating to survive the stomach transit. The largest activity gains in the variants were observed at low pH (pH 3) (data not shown). These improvements are not attributed to a true improvement in catalytic efficiency of the lipase, but instead to significant improvements in survivability.

This example demonstrates that the engineered lipase variants remain stable against proteolysis, withstand low acidity and harsh conditions of the stomach, and remain highly active against physiologically relevant fats (DHA) deficient in patients. The top engineered lipases met all goals and maintained high activity. It is believed that the increased survivability of the engineered lipases allows them to be immediately active to maximize fat hydrolysis, improve performance, and treat patients with deficits in fat hydrolysis, including those who have not responded to standard-of-care treatments.

### Example 9: Dosing Study for Lipase Variant V325

This example describes a dosing study to support dose selection for use in clinical studies with patients with exocrine pancreatic insufficiency (EPI) or malabsorption.

The dosing study used a pig model for EPI, which is an established surgical model of pancreatic insufficiency used to study the uptake of macronutrients and to evaluate different preparations of orally administered pancreatic enzymes (Donaldson et al. (2009) ADV. MED. SCI. 54(1):7-13; Pierzynowska et al. (2018) ARCH. MED. SCI. 14(2):407-414; Freedman et al. (2004) N. ENGL. J. MED. 350(6):560-9, Abello et al. (1989) PANCREAS 4(5):556-64).

The EPI pig model was selected because humans and pigs share many similarities functionally and developmentally with regard to the gastrointestinal tract, genitourinary structures and development of brain and pancreas (Gonzalez et al. (2015) TRANSL RES. 166(1):12-27; Luu et al. (2020) BMC GASTROENTEROL 20:403). A comparison of the recommended daily allowances of vitamins and minerals in the human diet and the daily nutrient requirement of pigs reveal similarities between the two species. The EPI porcine model appears to be well suited to evaluate native porcine enzymes (pancreatin, pancrelipase) and their role in exocrine pancreatic insufficiency. The EPI porcine model has also been adapted for testing the efficacy of microbially derived enzymes (Grujic et al. (2015) “The Long Term Positive Effect of G-Tube Feeding with an In-Line Enzyme Cartridge (EFIC) on the Tissue Levels of DHA and EPA in Pig Model of Exocrine Pancreatic Insufficiency (EPI)”, PEDIATRIC PULMONOLOGY 50:405-406).

Exocrine pancreatic insufficiency in pigs is achieved by ligation of the accessory exocrine pancreatic duct, which serves as the main pancreatic duct that drains pancreatic juices into the duodenum. Surgical ligation dramatically reduces the levels of digestive enzymes released into the duodenum, causing a reduction in fat, protein, and carbohydrate digestion and absorption. In addition, duodenal pH is also reduced, as in humans with EPI, producing another negative effect for enzyme activity in the gut lumen. (Martin et al. (2014) “A novel point-of-care lipase (ALCT-460) increases fat hydrolysis and omega 3 fat absorption in pics with exocrine pancreatic insufficiency,” JOURNAL OF CYSTIC FIBROSIS 13(Supplement 2): S58; Martin et al. (2014) “Increased Total Fat and Long Chain Polyunsaturated Fatty Acid Absorption in Pigs with Exocrine Pancreatic Insufficiency Fed a Formula Pre-Hydrolyzed with a Novel Point-of Care Lipase (ALCT-460), PEDIATRIC PULMONOLOGY 49:408.) The increased acidity in the small intestine also can provoke bile acid precipitation that affects micelle formation and lipid absorption. All of these observations agree with the results observed in humans with EPI (Corring et al. (1977) J. NUTR. 107(7):1216-21, Lankisch (1993) DIGESTION 54 Suppl 2:21-9).

The EPI pig model was used to evaluate specific measures of macronutrient absorption by assessing the byproducts of digestion (e.g., fatty acids and monoglycerides for lipase) and their uptake in plasma and tissues (erythrocytes, enterocytes). Evidence from the EPI pig model provides substantial support of the safety, efficacy and stability of the V325 lipase in combination with protease and amylase.

### Experimental Design

Surgery was performed on eighteen (n=18) juvenile pigs to induce EPI. The study Treatment Period included twelve (n=12) juvenile EPI pigs. Development of EPI was confirmed by arrested growth and steatorrhea. The twelve EPI pigs included in the Treatment Period were selected based on degree of steatorrhea and weight. The pigs weighed approximately 10±2 kg each. Pigs were fed 4% of their body weight with approximately 1% of body weight during the morning meal and approximately 3% of body weight during the afternoon meal.

The study included two test periods.
Experiment One was a dosing study to support dose selection for human patients with EPI or malabsorption.
Experiment Two selected 6 pigs from Experiment One to continue. Experiment Two evaluated lipase, protease and amylase release characteristics and activity as measured by evaluating the by-products of digestion in chyme analyzed by placing cannulas in the stomach, duodenum and proximal ileum.

### Experiment One—Protocol

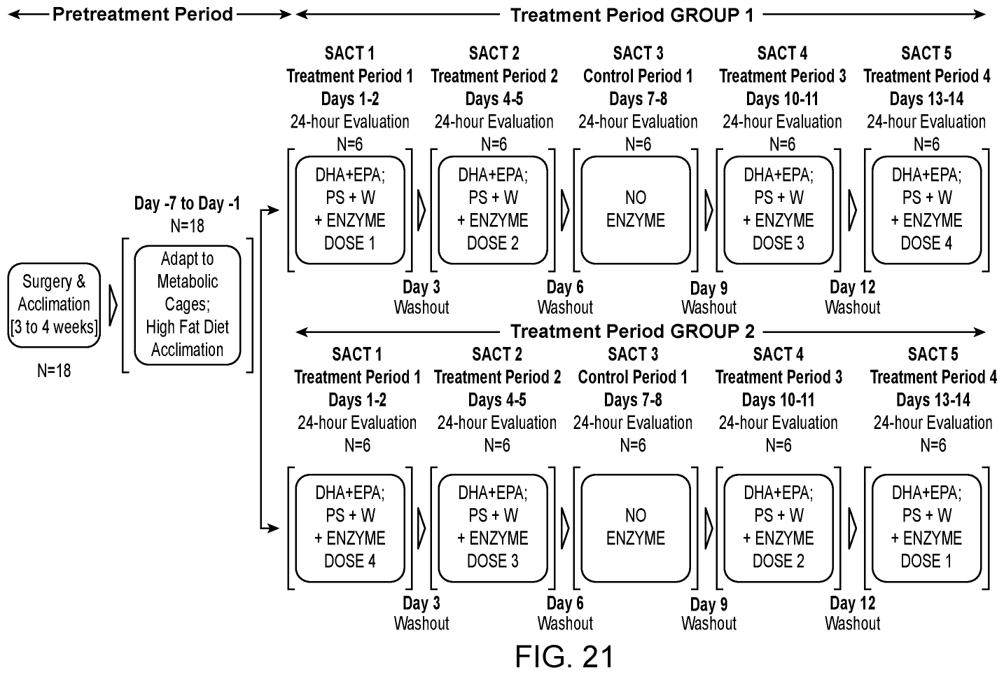

The study design contained five blocks, each block proceeding over three days to facilitate testing of varying V325 lipase doses given with protease and amylase in conjunction with a standard human diet to evaluate absorption. A schematic of treatment group design is provided in FIG. 21. As shown, on days 1, 4, 7, 10 and 13, pig received the V325 lipase and the selected substrate (4 g DHA and EPA triglycerides (“DHA+EPA”), 10 g whey (“W”) and 20 g potato starch (“PS”)). After 24 hours (i.e., on days 2, 5, 8, 11, and 14) blood collection was taken, and after 48 hours (i.e., on days 3, 6, 9, 12, and 15) a second blood collection was taken. For group 1, dose 1 was given on day 1, dose 2 was given on day 4, dose 3 was given on day 10, and dose 4 was given on day 13. For group 2, dose 4 was given on day 1, dose was 3 given on day 4, dose 2 was given on day 10 and dose 1 was given on day 13. Days 3, 6, 9 and 12 represented washout days before beginning the next treatment. The primary objectives were to evaluate safety, dosing and performance of the V325 lipase in combination with protease, and amylase.

A substrate absorption challenge test (SACT) evaluated clinical biomarkers of absorption directly related to byproducts of hydrolysis for individual substrates of fat, protein and starch in a well-controlled standardized testing environment. Absorption was assessed using varying doses of V325 lipase. The SACTs can provide a measure of intraluminal (small intestine: duodenal, jejunum, ileum) enzymatic activity and a direct assessment of nutrient absorption through the gastrointestinal lumen. In the SACTs, the V325 lipase was administered orally with a fixed amount of food (see above) and substrate (4 g DHA and EPA triglycerides) and the product(s) of the lipolysis reaction upon absorption (e.g., DHA and EPA fatty acids, with 24 fatty acids in total) were monitored in the blood.

For measurement of lipase activity, the triglycerides of docosahexaenoic acid (DHA) and eicosapentaenoic acid (EPA) were used as the SACT challenge substrate because, due to their chain-lengths as well as the numbers of double-bonds, they are among the most difficult dietary fats to digest and absorb (Burdge et al. (2005) REPROD. NUTR. DEV. 45:581-597; Hussein et al. (2005) JOURNAL OF LIPID RESEARCH 46:269-280.) DHA and EPA are clinically meaningful fatty acids, biologically relevant, and important for growth and development. The use of DHA and EPA triglycerides as the substrate in the SACT to evaluate in vivo lipase activity (lipolysis) allows for the measurement of their breakdown products (DHA and EPA fatty acids) in the blood over 24-hours using a validated gas chromatography-flame ionization detector (GC-FID) method (OmegaQuant, South Dakota). Furthermore, since the endogenous conversion of essential fatty acids to EPA and DHA is extremely limited, these fatty acids are unique direct absorption biomarkers to measure the effectiveness of exogenously administered V325 lipase. The DHA/EPA challenge test directly evaluates the ability of an exogenously administered lipase to digest long chain polyunsaturated fatty acids (LCPUFAs), a stringent test of lipolysis, and the absorption readouts over time are reliable pharmacokinetic measurements, including Cₘₐₓ, Tₘₐₓ and AUC for DHA and EPA. Fatty acids, specifically DHA and EPA in plasma and erythrocytes, correlate strongly with dietary fat intake and are a biomarker for overall fat absorption in people with CF.

The SACT was performed by administering pills containing omega-3 triglycerides from fish oil (˜4 g DHA and EPA) to the pigs and collecting a small volume of blood 6-8 times over 24-hours as shown in TABLE 18 (see also, e.g., Freedman et al. (2004) N. ENGL. J. MED. 350(6):560-9).

TABLE 18

| Enzyme | Blood collection time points for biomarker analysis |
|---|---|
| Lipase | 0, 1, 2, 4, 6, 8, 12, 24, 48 hours |
| Protease | 0, 15, 30 min; 1, 2, 4, 6, 8, 12, 24 hours |
| Amylase | 0, 15, 30, 45, 60, 75, 90, 120, 180, 240 minutes |

Fat absorption in plasma was determined by evaluating the area under the curve (AUC) and concentration peak (Cₘₐₓ) or time of peak concentration (Tₘₐₓ) over 24 hours. Thus, changes due to V325 lipase (or comparator) were measured in a standardized manner. In addition to EPA (20:5n-3) and DHA (22:6n-3), the following 22 fatty acids (by class) were measured:
a) saturated (14:0, 16:0, 18:0, 20:0, 22:0 24:0),
b) monounsaturated (16:1, 18:1, 20:1, 24:1),
c) trans unsaturated (16:1, 18:1, 18:2),
d) n-6 polyunsaturated (18:2, 18:3, 20:2, 20:3, 20:4, 22:4, 22:5), and
e) n-3 polyunsaturated (18:3, 22:5).

The sum of these 24 fatty acids constitutes the total fatty acid content of the blood, and each individual fatty acid can be expressed as a percent of the total or as a concentration (e.g., μg/mL). As shown in FIG. 21, each SACT period provided an evaluation of plasma uptake corresponding to each V325 lipase dose compared to a period with no enzyme. There were five (5) SACT periods during this experiment: four (4) lipase doses, 20 mg, 40 mg, 80 mg, 120 mg, and a no enzyme period.

TABLE 19
Enzyme dose in the study during treatment

| | V325 Lipase | Protease | Amylase |
|---|---|---|---|
| Dose | Dose (mg) | Dose (mg) | Dose (mg) |
| 1 | 20 | 25 | 40 |
| 2 | 40 | 50 | 80 |
| 3 | 80 | 50 | 120 |
| 4 | 120 | 75 | 160 |

### Results Experiment One

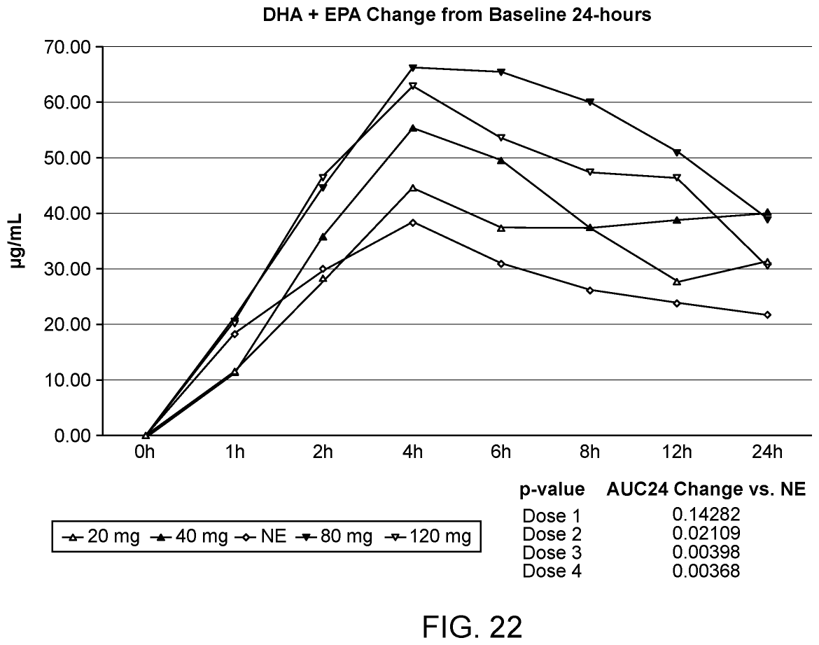

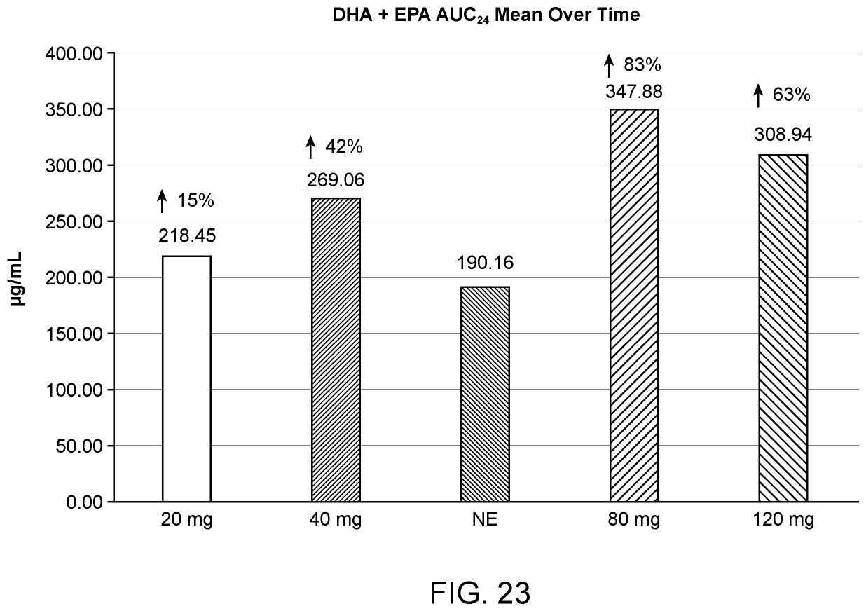

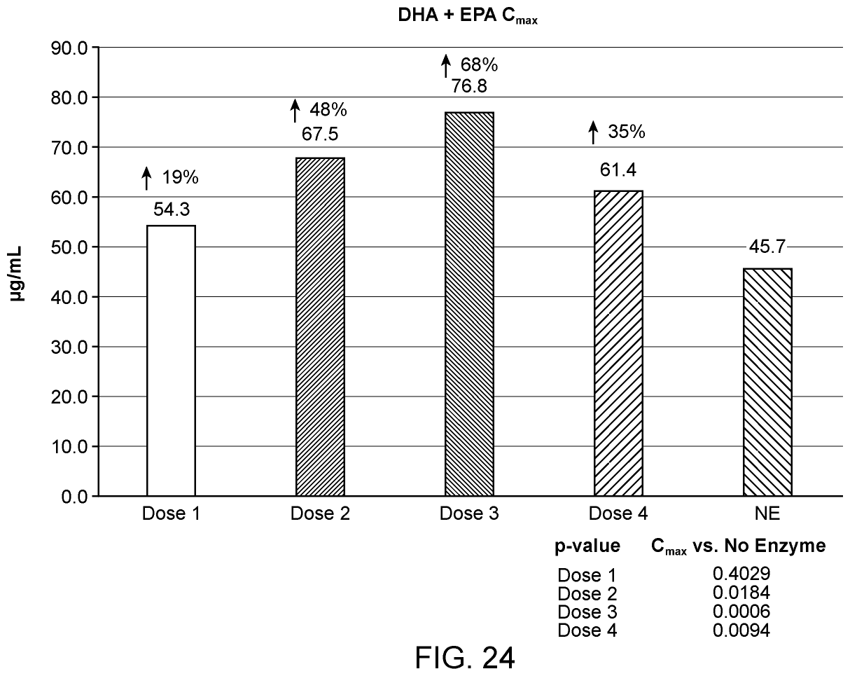

As shown in FIG. 22, V325 lipase demonstrated a significantly higher AUC and Cₘₐₓ compared to control (no enzyme (“NE”)+substrate) with a Tₘₐₓ at ˜4 hours. Escalating V325 lipase (40 mg, 80 mg, 120 mg) demonstrated a significantly higher uptake of DHA+EPA over 24-hours compared to control (40 mg p=0.02, 80 mg p=0.04, 120 mg p=0.03). AUC₂₄ mean over time is shown in FIG. 23 (40 mg=42%, 80 mg=83%, 120 mg=63%). As shown in FIG. 24, baseline subtracted Cₘₐₓ was significantly higher for the 40 mg, 80 mg and 120 mg dosages of V325 lipase compared to no enzyme (p=0.02, 0.0006, 0.009 respectively).

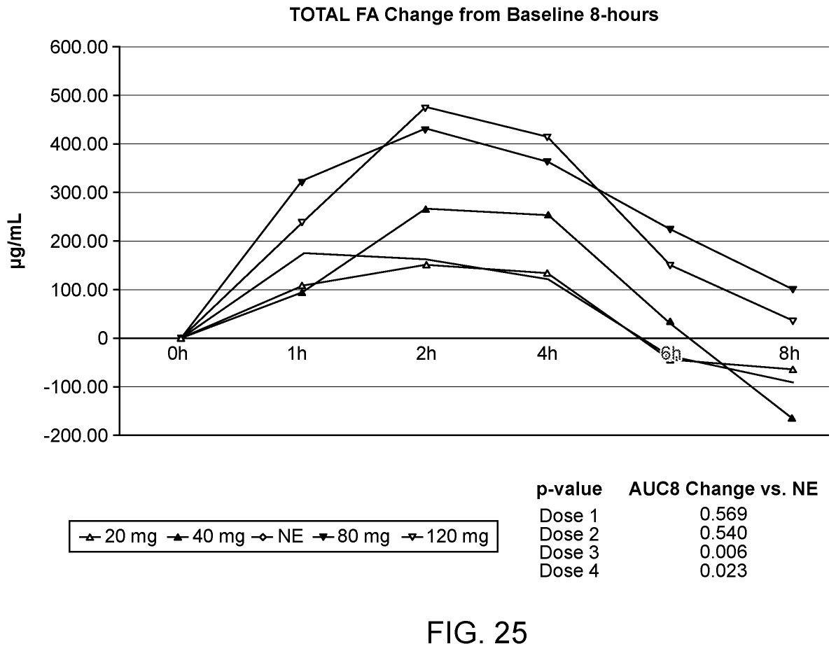

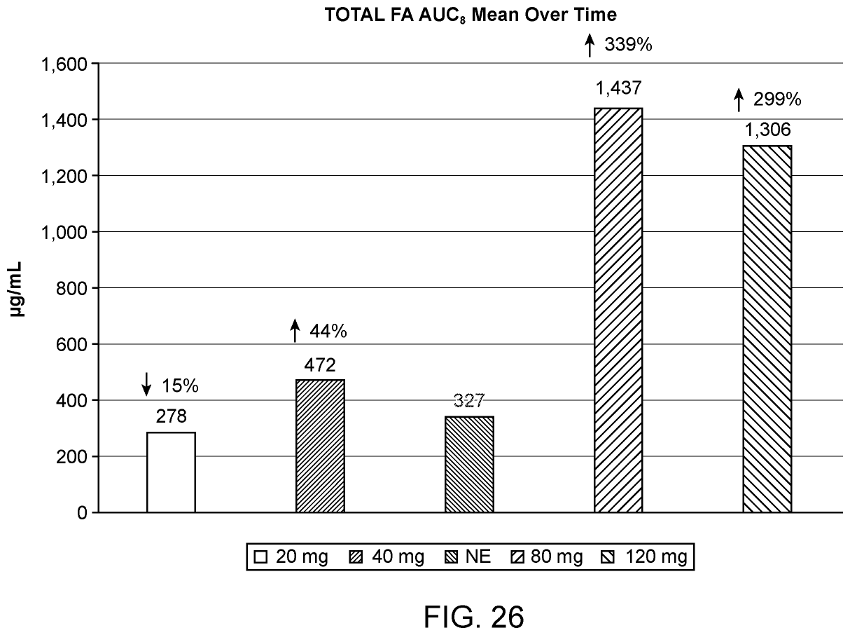

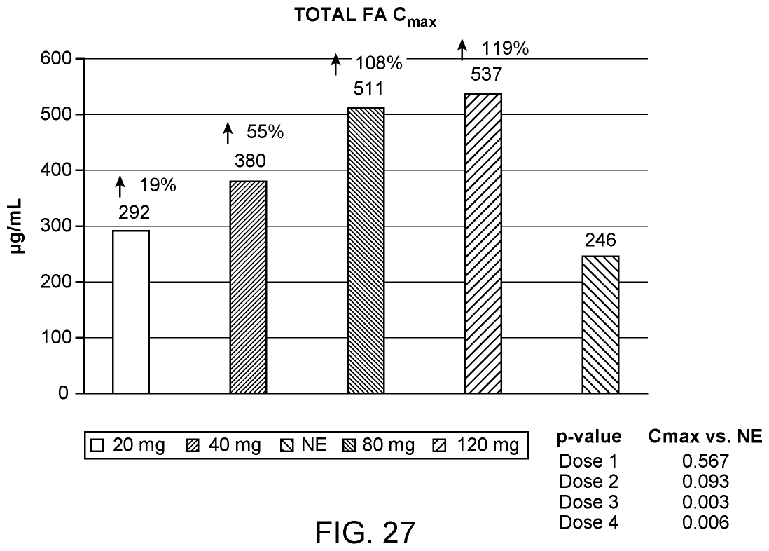

A similar response was observed when evaluating total fatty acids (FA) (n=24 total fatty acids). As shown in FIG. 25, V325 lipase AUC and Cₘₐₓ were significantly higher when compared to control (no enzyme+substrate). Escalating V325 lipase doses (20 mg, 40 mg, 80 mg, 120 mg) demonstrated higher uptake of total fatty acids over 8-hours compared to control (FIG. 25). AUC and Cₘₐₓ were approximately 2-3 fold greater with higher V325 doses (80 mg, 120 mg) when compared to control (no enzyme). AUC for V325 doses (80 mg, 120 mg) were significantly increased (p=0.006, 0.02) when compared to no enzyme. AUC₂₄ mean over time for total fatty acids is shown in FIG. 26 (40 mg=42%, 80 mg=83%, 120 mg=63%). As shown in FIG. 27, Cₘₐₓ for V325 doses (80 mg, 120 mg) were significantly increased (p=0.003, 0.006) when compared to no enzyme. Similar results were seen for common dietary fats oleic, palmitic, stearic and elaidic (OPSE); saturated fatty acids; and beneficial fatty acids (DHA+EPA+docosapentaenoic acid (DPA)) (data not shown).

### Experiment Two—Protocol

Both chyme and plasma markers of absorption were evaluated in Experiment Two. Tests for plasma absorption were performed as in Experiment One.

Chyme was used to evaluate enzyme activity as measured by release of oleic acid, while plasma biomarkers of fatty acids were used to evaluate the end products of triglyceride digestion. Oleic acid is common dietary fat and major portion of olive oil which has historically been used in the lipase USP method. Chyme is the thick semifluid mass of partly digested food that is passed from the stomach to the duodenum and through the small intestine. Chyme contains natural post-meal conditions (e.g., pH, meal content, bile salts, micronutrient interplay) and is a highly relevant dietary substrate to evaluate lipase activity, stability and performance as measured by the release of lipolysis byproducts (e.g., fatty acids of oleic acid).

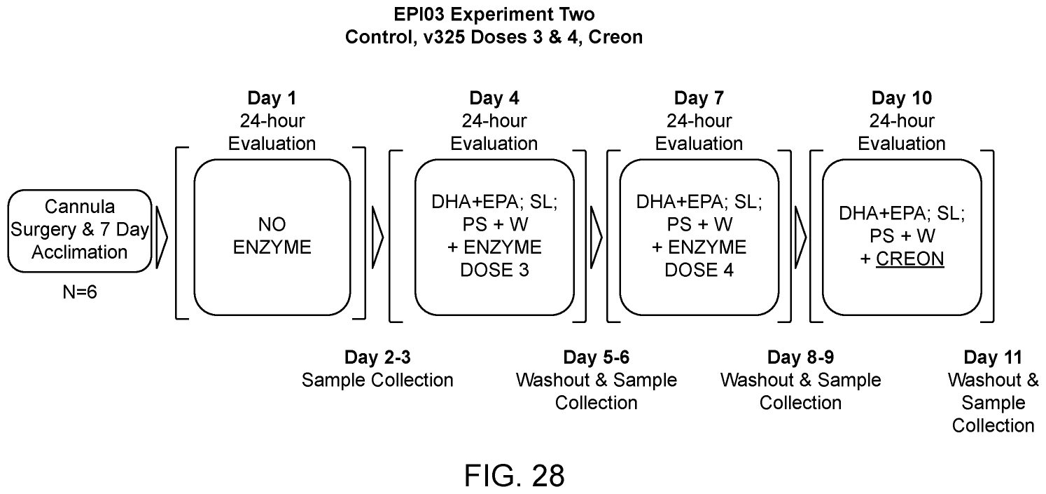

During this experiment, a commercially available porcine enzyme product (Creon® pancrelipase) was also used as a comparator. FIG. 28 provides a schematic of the experimental protocol. There were four (4) testing periods: two V325 lipase doses (80 mg, 120 mg), Creon® 50,000 units, and a no enzyme period. The EPI model was originally developed and optimized for evaluation of porcine extracts (pancreatin, pancrelipase) so it was expected that EPI pigs would show improved performance, because Creon® pancrelipase includes native porcine enzymes. The maximal human recommended for people with cystic fibrosis for Creon® was used a comparator (2,500 kg body weight).

Chyme was collected at various positions in the digestive tract and the time points shown in TABLE 20.

TABLE 20
Time of chyme collections after enzyme administration

| Minutes | Stomach | Duodenum | Ileum |
|---|---|---|---|
| 5 | X | X | |
| 15 | X | X | X |
| 30 | X | X | X |
| 60 | X | X | X |
| 90 | X | X | X |
| 120 | X | X | X |
| 180 | X | X | X |
| 240 | X | X | X |
| 300 | | X | X |
| 360 | | X | X |

Lipase activity measured lipolysis, specifically the release of oleic acid (c:18:1 n-9) in chyme (μM/minute/mL chyme). Release of oleic acid was assessed using a colorimetric method (Lipase Detection Kit ab102524, Abcam, UK) at a physiologically relevant pH of 6.0.

### Results Experiment Two

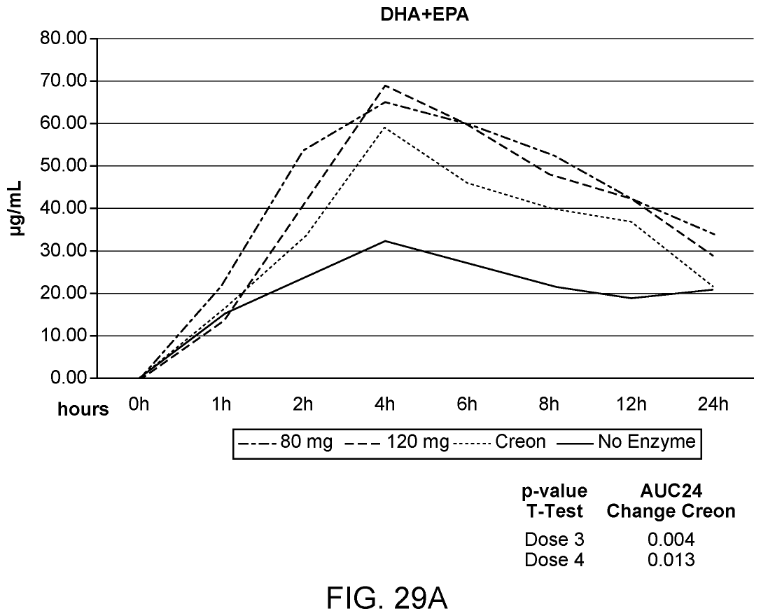

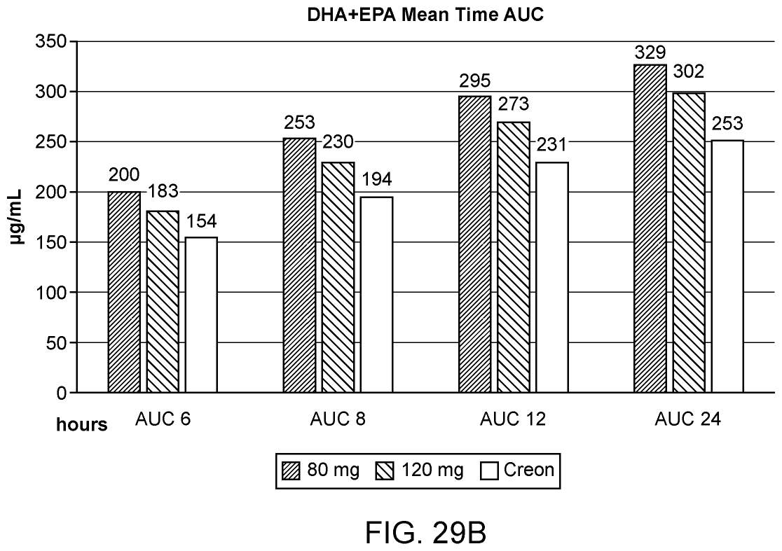

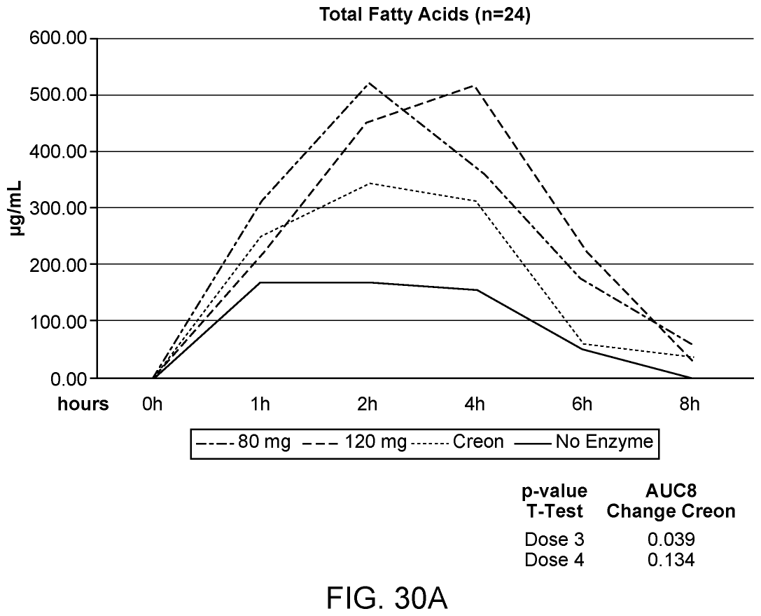

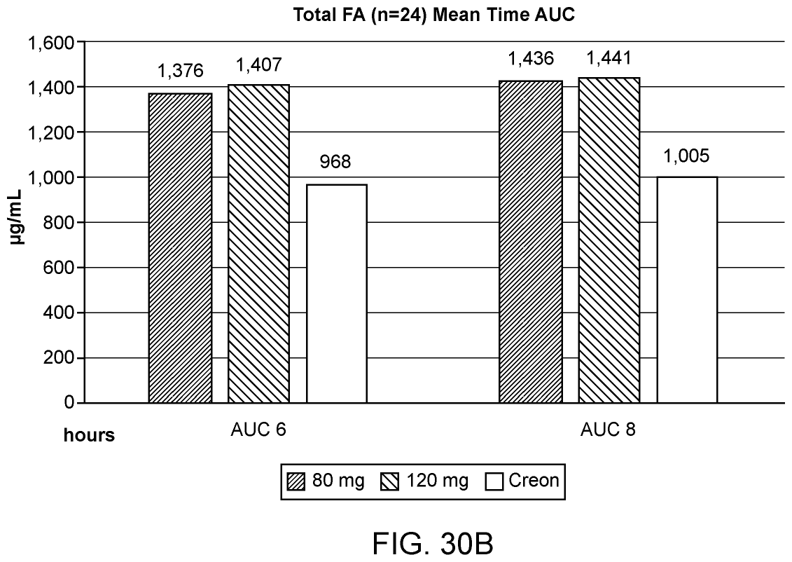

Plasma: As shown in FIG. 29A, V325 lipase demonstrated significantly higher AUC and Cₘₐₓ of DHA+EPA when compared to Creon® (pancrelipase) and control (NE: no enzyme). V325 lipase demonstrated earlier uptake Tₘₐₓ with higher overall AUC and Cₘₐₓ than Creon® or control. As shown in FIG. 29B, V325 demonstrated a 20-30% increase in DHA+EPA mean change for AUC when compared to Creon®. These changes in response were consistent from 6 to 24-hours. Similar results were observed for total fatty acids (see, FIGS. 30A and 30B).

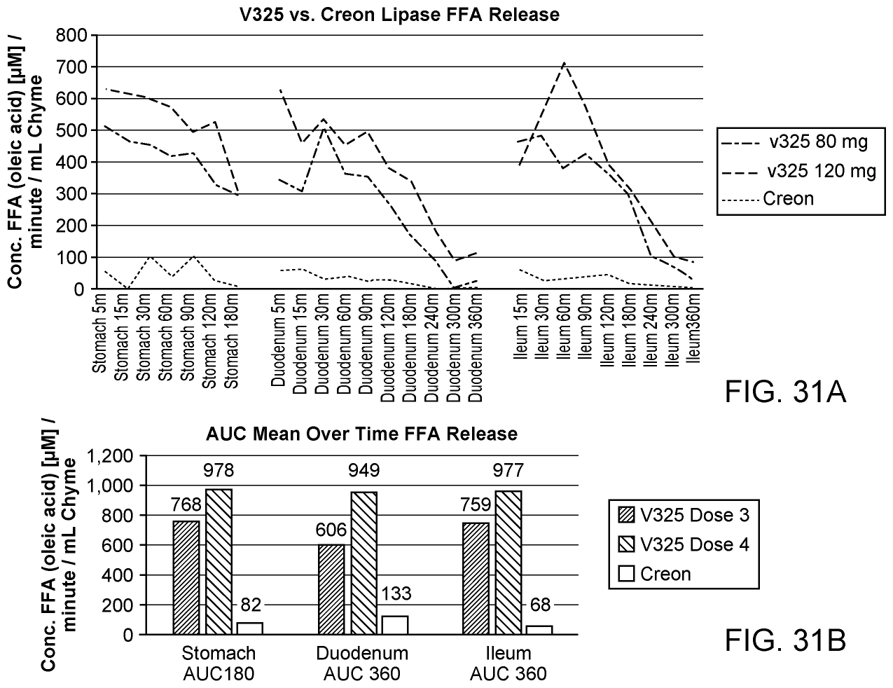

V325 activity and stability: As shown in FIG. 31, V325 lipase demonstrated significantly higher and earlier release of fatty acids (oleic acid) in each GI compartment tested (stomach, duodenum, ileum) compared to Creon®. Additionally, and importantly, V325 lipase doses demonstrated similar fatty acid release profiles in each compartment demonstrating the stability of the V325 lipase in vivo. V325 lipase begins to digest fats immediately in the stomach and duodenum and has sustained activity throughout GI compartments of interest confirming enzyme engineering stability in a physiological environment. Understanding where fat is cleaved by lipase informs how they are absorbed, as well as for dose selection. Evaluating enzyme activity in chyme allows for the assessment of activity in natural post-meal conditions (e.g., pH, meal content, bile salts, micronutrient interplay). The lipase Cmax was similar in each compartment tested, supporting the stability of the V325 lipase.

Earlier release and absorption, as demonstrated with V325, allows for more physiological absorption in the upper small intestine. The primary absorption transporters appear to be located in the upper small intestine so late enzyme release or poor stability does not allow for physiological absorption. Furthermore, chyme viscosity increases significantly between the duodenum to the ileum. Thus, the earlier lipolysis demonstrated by V325 allows for greater mixing and improved performance.

This example demonstrates that administration of the V325 lipase results in a significantly higher release of fatty acids (oleic acid) compared to Creon® 50,000 at pH 6.0. In addition, the earlier release of fatty acids by the V325 lipase and its stability throughout the important compartments of the stomach and small intestine is evidence that the V325 lipase provides improved performance compared not only to the no enzyme control, but also to the standard of care (Creon®).

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A recombinant mutant microbial lipase enzyme, wherein the lipase comprises one or more of (i) increased stability at acidic pH (e.g., pH 3.0 or 4.0) relative to a corresponding wild-type microbial lipase enzyme, (ii) increased stability in the presence of a protease (e.g., a serine protease and/or an aspartic protease) relative to the corresponding wild-type microbial lipase enzyme, or (iii) at least 60%, 70%, 80%, 85%, 90%, 95%, 96%, 97%, 98%, 99%, or 100% of the enzymatic activity of the corresponding wild-type microbial lipase enzyme.

2. The lipase of claim 1, wherein the lipase comprises:
(a) a substitution of a residue at a position corresponding to position 102 of wild-type B. cepacia lipase;
(b) a substitution of a residue at a position corresponding to position 125 of wild-type B. cepacia lipase;
(c) a substitution of a residue at a position corresponding to position 137 of wild-type B. cepacia lipase;
(d) a substitution of a residue at a position corresponding to position 153 of wild-type B. cepacia lipase;
(e) a substitution of a residue at a position corresponding to position 154 of wild-type B. cepacia lipase;
(f) a substitution of a residue at a position corresponding to position 221 of wild-type B. cepacia lipase;
(g) a substitution of a residue at a position corresponding to position 227 of wild-type B. cepacia lipase;
(h) a substitution of a residue at a position corresponding to position 249 of wild-type B. cepacia lipase;
(i) a substitution of a residue at a position corresponding to position 266 of wild-type B. cepacia lipase;
(j) a substitution of a residue at a position corresponding to position 300 of wild-type B. cepacia lipase;
(k) a substitution of a residue at a position corresponding to position 39 of wild-type Burkholderia cepacia lipase;
(l) a substitution of a residue at a position corresponding to position 79 of wild-type B. cepacia lipase;
(m) a substitution of a residue at a position corresponding to position 128 of wild-type B. cepacia lipase;
(n) a substitution of a residue at a position corresponding to position 138 of wild-type B. cepacia lipase;
(o) a substitution of a residue at a position corresponding to position 161 of wild-type B. cepacia lipase;
(p) a substitution of a residue at a position corresponding to position 170 of wild-type B. cepacia lipase;
(q) a substitution of a residue at a position corresponding to position 240 of wild-type B. cepacia lipase;
(r) a substitution of a residue at a position corresponding to position 250 of wild-type B. cepacia lipase;
(s) a substitution of a residue at a position corresponding to position 260 of wild-type B. cepacia lipase;
(t) a substitution of a residue at a position corresponding to position 281 of wild-type B. cepacia lipase;
or a combination of any of the foregoing substitutions.

3. The lipase of claim 2, wherein, in the lipase:
(a) the residue at a position corresponding to position 102 of wild-type B. cepacia lipase is substituted by Q;
(b) the residue at a position corresponding to position 125 of wild-type B. cepacia lipase is substituted by S;
(c) the residue at a position corresponding to position 137 of wild-type B. cepacia lipase is substituted by A;
(d) the residue at a position corresponding to position 153 of wild-type B. cepacia lipase is substituted by N;
(e) the residue at a position corresponding to position 154 of wild-type B. cepacia lipase is substituted by H;
(f) the residue at a position corresponding to position 221 of wild-type B. cepacia lipase is substituted by L;
(g) the residue at a position corresponding to position 227 of wild-type B. cepacia lipase is substituted by K;
(h) the residue at a position corresponding to position 249 of wild-type B. cepacia lipase is substituted by L;
(i) the residue at a position corresponding to position 266 of wild-type B. cepacia lipase is substituted by L;
(j) the residue at a position corresponding to position 300 of wild-type B. cepacia lipase is substituted by Y;
(k) the residue at a position corresponding to position 39 of wild-type B. cepacia lipase is substituted by R;
(l) the residue at a position corresponding to position 79 of wild-type B. cepacia lipase is substituted by Q;
(m) the residue at a position corresponding to position 128 of wild-type B. cepacia lipase is substituted by N;
(n) the residue at a position corresponding to position 138 of wild-type B. cepacia lipase is substituted by I;
(o) the residue at a position corresponding to position 161 of wild-type B. cepacia lipase is substituted by A;
(p) the residue at a position corresponding to position 170 of wild-type B. cepacia lipase is substituted by S;
(q) the residue at a position corresponding to position 240 of wild-type B. cepacia lipase is substituted by V;
(r) the residue at a position corresponding to position 250 of wild-type B. cepacia lipase is substituted by A;
(s) the residue at a position corresponding to position 260 of wild-type B. cepacia lipase is substituted by A;
(t) the residue at a position corresponding to position 281 of wild-type B. cepacia lipase is substituted by A;
or the lipase comprises a combination of any of the foregoing substitutions.

4. The lipase of any one of claims 1-3, wherein the lipase comprises:
(a) a substitution of a D residue at a position corresponding to position 102 of wild-type B. cepacia lipase (D102);
(b) a substitution of a G residue at a position corresponding to position 125 of wild-type B. cepacia lipase (G125);
(c) a substitution of a T residue at a position corresponding to position 137 of wild-type B. cepacia lipase (T137);
(d) a substitution of an S residue at a position corresponding to position 153 of wild-type B. cepacia lipase (S153);
(e) a substitution of an N residue at a position corresponding to position 154 of wild-type B. cepacia lipase (N154);
(f) a substitution of an F residue at a position corresponding to position 221 of wild-type B. cepacia lipase (F221);
(g) a substitution of a T residue at a position corresponding to position 227 of wild-type B. cepacia lipase (T227);
(h) a substitution of an F residue at a position corresponding to position 249 of wild-type B. cepacia lipase (F249);
(i) a substitution of a V residue at a position corresponding to position 266 of wild-type B. cepacia lipase (V266);
(j) a substitution of an N residue at a position corresponding to position 300 of wild-type B. cepacia lipase (N300);
(k) a substitution of a Q residue at a position corresponding to position 39 of wild-type Burkholderia cepacia lipase (Q39);
(l) a substitution of a T residue at a position corresponding to position 79 of wild-type B. cepacia lipase (T79);
(m) a substitution of an A residue at a position corresponding to position 128 of wild-type B. cepacia lipase (A128);
(n) a substitution of a V residue at a position corresponding to position 138 of wild-type B. cepacia lipase (V138);
(o) a substitution of an L residue at a position corresponding to position 161 of wild-type B. cepacia lipase (L128);
(p) a substitution of an A residue at a position corresponding to position 170 of wild-type B. cepacia lipase (A170);
(q) a substitution of an A residue at a position corresponding to position 240 of wild-type B. cepacia lipase (A240);
(r) a substitution of a G residue at a position corresponding to position 250 of wild-type B. cepacia lipase (G250);
(s) a substitution of an S residue at a position corresponding to position 260 of wild-type B. cepacia lipase (S260);
(t) a substitution of an S residue at a position corresponding to position 281 of wild-type B. cepacia lipase (S281);
or a combination of any of the foregoing substitutions.

5. The lipase of claim 4, wherein, in the lipase:
(a) the D residue at a position corresponding to position 102 of wild-type B. cepacia lipase is substituted by Q (D102Q);
(b) the G residue at a position corresponding to position 125 of wild-type B. cepacia lipase is substituted by S (G125S);
(c) the T residue at a position corresponding to position 137 of wild-type B. cepacia lipase is substituted by A (T137A);
(d) the S residue at a position corresponding to position 153 of wild-type B. cepacia lipase is substituted by N (S153N);
(e) the N residue at a position corresponding to position 154 of wild-type B. cepacia lipase is substituted by H (N154H);
(f) the F residue at a position corresponding to position 221 of wild-type B. cepacia lipase is substituted by L (F221L);
(g) the T residue at a position corresponding to position 227 of wild-type B. cepacia lipase is substituted by K (T227K);
(h) the F residue at a position corresponding to position 249 of wild-type B. cepacia lipase is substituted by L (F249L);
(i) the V residue at a position corresponding to position 266 of wild-type B. cepacia lipase is substituted by L (V266L);
(j) the N residue at a position corresponding to position 300 of wild-type B. cepacia lipase is substituted by Y (N300Y);
(k) the Q residue at a position corresponding to position 39 of wild-type B. cepacia lipase is substituted by R (Q39R);
(l) the T residue at a position corresponding to position 79 of wild-type B. cepacia lipase is substituted by Q (T79Q);
(m) the A residue at a position corresponding to position 128 of wild-type B. cepacia lipase is substituted by N (A128N);
(n) the V residue at a position corresponding to position 138 of wild-type B. cepacia lipase is substituted by I (V1381);
(o) the L residue at a position corresponding to position 161 of wild-type B. cepacia lipase is substituted by A (L161A);
(p) the A residue at a position corresponding to position 170 of wild-type B. cepacia lipase is substituted by S (A170S);
(q) the A residue at a position corresponding to position 240 of wild-type B. cepacia lipase is substituted by V (A240V);
(r) the G residue at a position corresponding to position 250 of wild-type B. cepacia lipase is substituted by A (G250A);
(s) the S residue at a position corresponding to position 260 of wild-type B. cepacia lipase is substituted by A (S260A);
(t) the S residue at a position corresponding to position 281 of wild-type B. cepacia lipase is substituted by A (S281A);
or the lipase comprises a combination of any of the foregoing substitutions.

6. The lipase of any one of claims 1-5, wherein the lipase comprises one, two, three, four, five, six, seven, eight, nine, ten, or more than ten mutations relative to the corresponding wild-type microbial lipase.

7. The lipase of claim 5 or 6, wherein the lipase comprises:
(a) the D102Q, N154H, and F221L substitutions;
(b) the D102Q, G125S, N154H, F221L, V266L, and N300Y substitutions;
(c) the T79Q, D102Q, G125S, T137A, N154H, F221L, T227K, F249L, V266L, and N300Y substitutions;
(d) the T79Q, D102Q, G125S, T137A, N154H, F221L, T227K, V266L, S281A, and N300Y substitutions;
(e) the T79Q, D102Q, G125S, S153N, N154H, F221L, T227K, V266L, S281A, and N300Y substitutions;
(f) the T79Q, D102Q, G125S, S153N, N154H, F221L, F249L, G250A, V266L, and N300Y substitutions;
(g) the T79Q, D102Q, G125S, S153N, N154H, F221L, F249L, V266L, S281A, and N300Y substitutions;
(h) the T79Q, D102Q, G125S, N154H, F221L, T227K, F249L, V266L, S281A, and N300Y substitutions;
(i) the D102Q, G125S, T137A, S153N, N154H, F221L, T227K, F249L, V266L, and N300Y substitutions;
(j) the D102Q, G125S, T137A, S153N, N154H, F221L, T227K, G250A, V266L, and N300Y substitutions;
(k) the D102Q, G125S, T137A, N154H, F221L, T227K, G250A, V266L, S281A, and N300Y substitutions;
(l) the D102Q, G125S, S153N, N154H, F221L, T227K, F249L, G250A, V266L, and N300Y substitutions; or
(m) the D102Q, G125S, S153N, N154H, F221L, T227K, F249L, V266L, S281A, and N300Y substitutions.

8. The lipase of any one of claims 1-7 wherein the lipase is a α/β-hydrolase lipase.

9. The lipase of any one of claims 1-8, wherein the lipase comprises a serine-histidine-aspartate active triad.

10. The lipase of any one of claims 1-9, wherein the lipase comprises a hydrophobic lid that opens to allow for the binding and/or hydrolysis of a lipid.

11. The lipase of claim 10, wherein the hydrophobic lid opens sufficiently to allow for the binding and/or hydrolysis of a triglyceride having a chain length of greater than eight carbons.

12. The lipase of any one of claims 1-11, wherein the lipase comprises a calcium binding site, wherein, when calcium is bound to the calcium binding site, the lipase is stabilized.

13. The lipase of any one of claims 1-12, wherein the lipase comprises an oxyanion hole, wherein the oxyanion hole stabilizes a negatively charged intermediate generated during fatty acid bond hydrolysis.

14. The lipase of any one of claims 1-13, wherein the lipase is a Family 1 bacterial lipase.

15. The lipase of claim 14, wherein the lipase is a I.1, I.2, or I.3 subfamily bacterial lipase.

16. The lipase of claim 15, wherein the lipase is a I.1 or I.2 subfamily bacterial lipase.

17. The lipase of claim 16, wherein the lipase is a I.2 subfamily bacterial lipase.

18. The lipase of any one of claims 1-17, wherein the lipase is a Burkholderia, Pseudomonas, or Chromobacterium lipase.

19. The lipase of claim 18, wherein the lipase is a Burkholderia cepacia, Burkholderia glumae, Pseudomonas fluorescens, Pseudomonas aeruginosa, Pseudomonas luteola, or Chromobacterium viscosum lipase.

20. The lipase of claim 19, wherein the lipase is a Burkholderia cepacia lipase.

21. The lipase of any one of claims 1-20, wherein the lipase comprises a S residue at a position corresponding to position 87 of wild-type B. cepacia (S87), a D residue at a position corresponding to position 264 of wild-type B. cepacia (D264), and a H residue at a position corresponding to position 286 of wild-type B. cepacia (H286).

22. The lipase of any one of claims 1-21, wherein the lipase comprises the amino acid sequence of any one of SEQ ID NOs: 2-14, or an amino acid sequence that has at least 85%, 90%, 95%, 96%, 97%, 98%, or 99% sequence identity to any one of SEQ ID NOs: 2-14.

23. A recombinant mutant microbial lipase enzyme comprising a substitution, or combination of substitutions, listed in TABLE 1 or TABLE 2.

24. The lipase of any one of claims 1-23, wherein the lipase has a half-life of at least 75 minutes, 100 minutes, 125 minutes, 150 minutes, 175 minutes, 180 minutes, 185 minutes, 190 minutes, 195 minutes, or 200 minutes in the presence of a serine protease.

25. The lipase of any one of claims 1-24, wherein the lipase has at least 0.5 fold, 1 fold, 1.5 fold, 2 fold, 2.5 fold, or 3 fold higher stability in the presence of a serine protease, compared to the corresponding wild-type lipase.

26. The lipase of claim 24 or 25, wherein the serine protease is Aspergillus melleus protease.

27. The lipase of any one of claims 1-26, wherein the lipase has a half-life of at least 50 minutes, 75 minutes, 100 minutes, 125 minutes, 130 minutes, 135 minutes, 140 minutes, 145 minutes, or 150 minutes at about pH 3.0.

28. The lipase of any one of claims 1-27, wherein the lipase has at least 1.5 fold, 2 fold, 2.5 fold, or 3 fold higher stability at about pH 3.0, compared to the corresponding wild-type lipase.

29. The lipase of any one of claims 1-28 wherein the lipase has a half-life of at least 50 minutes, 75 minutes, 100 minutes, 125 minutes, 150 minutes, 175 minutes, 200 minutes, 225 minutes, 230 minutes, or 235 minutes in the presence of an aspartic protease (e.g., at pH 3.6).

30. The lipase of any one of claims 1-29, wherein the lipase has at least 1.5 fold, 2 fold, 2.5 fold, 3 fold, 3.5 fold, or 4 fold higher stability in the presence of an aspartic protease (e.g., at pH 3.6), compared to the corresponding wild-type lipase.

31. The lipase of claim 29 or 30, wherein the aspartic protease is pepsin.

32. The lipase of any one of claims 1-31 wherein the lipase has a half-life of at least 50 minutes, 75 minutes, 100 minutes, 125 minutes, 150 minutes, 175 minutes, 180 minutes, 185 minutes, 190 minutes, 195 minutes, or 200 minutes in the presence of pancreatin.

33. The lipase of any one of claims 1-32, wherein the lipase has at least 0.5 fold, 1 fold, 1.5 fold, 2 fold, 2.5 fold, or 3 fold higher stability in the presence of pancreatin, compared to the corresponding wild-type lipase.

34. The lipase of any one of claims 1-33, wherein the lipase has at least 0.5 fold, 1 fold, 1.5 fold, 2 fold, 2.5 fold, or 3 fold higher activity at about pH 3.0, compared to the corresponding wild-type lipase.

35. The lipase of any one of claims 1-34, wherein the lipase has a specific activity at pH 3.0 of at least 300, 400, 500, 600, 700, 800, 900, or 1,000 μmol fatty acids (FA) produced/min/mg of lipase towards a long-chain triglyceride substrate including 37% DHA triglyceride and 22% oleic acid triglyceride or triolein.

36. The lipase of any one of claims 1-35, wherein the lipase has a specific activity at pH 4.0, pH 5.0, or pH 6.0 of at least 600, 700, 800, 900, 1,000, 1,100, 1,200, 1,300, 1,400, 1,500, or 2,000 μmol fatty acids (FA) produced/min/mg of lipase towards a long-chain triglyceride substrate including 37% DHA triglyceride and 22% oleic acid triglyceride or triolein.

37. The lipase of any one of claims 1-36, wherein the lipase has a specific activity at pH 7.0 of at least 1,000, 1,100, 1,200, 1,300, 1,400, 1,500, or 2,000 μmol fatty acids (FA) produced/min/mg of lipase towards a long-chain triglyceride substrate including 37% DHA triglyceride and 22% oleic acid triglyceride or triolein.

38. The lipase of any one of claims 1-37, wherein the lipase preferentially hydrolyzes the sn-1 and sn-3 positions on a triglyceride.

39. The lipase of any one of claims 1-38, wherein the lipase enzymatic activity (e.g., specific activity) is not inhibited by bile salts.

40. The lipase of any one of claims 1-39, wherein the lipase does not require a colipase.

41. The lipase of any one of claims 1-40, wherein the lipase is not cross-linked and/or crystallized.

42. The lipase of any one of claims 1-41, wherein the lipase remains sufficiently active at a pH in the range of 3.5 to 7.0 to hydrolyze long-chain poly-unsaturated fatty acids (LCPUFAs), e.g., DHA and EPA, or long-chain triglycerides, e.g., oleic acid or triolein, in the gastrointestinal tract of a subject.

43. The lipase of claim 42, wherein the lipase is at least 2 fold, 10 fold, 100 fold or 1,000 fold more active than pancrelipase when tested under the same conditions.

44. The lipase of any one of claims 1-43, wherein more than 50%, 60%, 70%, 80%, or 90% of the lipase remains active in the fed-state stomach of a subject for 60-120 minutes.

45. The lipase of any one of claims 1-44, wherein the lipase digests greater than 20%, 30%, 40%, or 50% of ingested fats in the stomach of a subject to fatty acids and monoglycerides.

46. The lipase of any one of claims 1-45, wherein more than 50%, 60%, 70%, 80%, or 90% of the lipase remains active through the small intestine of a subject from about 240 to about 360 minutes.

47. The lipase of any one of claims 1-46, wherein the lipase digests greater than 50%, 60%, 70%, 80%, or 90% of ingested fats in the small intestine of a subject to fatty acids and monoglycerides.

48. The lipase of any one of claims 1-47, wherein the lipase increases absorption of long-chain unsaturated fatty acids to the plasma in a subject within 30 minutes, 45 minutes, 60 minutes, 90 minutes, or 120 minutes by more than 25%, 35%, 50%, 100%, or 200% relative to the same subject when that subject has not been administered the lipase, or relative to a similar subject that has not been administered the lipase.

49. The lipase of any one of claims 1-48, wherein the lipase increases absorption of fat-soluble vitamins (e.g., vitamin A, vitamin D, vitamin E, vitamin K).

50. The lipase of any one of claims 1-49, wherein the lipase increases absorption of choline.

51. A nucleic acid encoding the lipase of any one of claims 1-50.

52. An expression vector comprising a nucleic acid sequence of claim 51.

53. The expression vector of claim 52, wherein the nucleic acid sequence encoding the recombinant mutant lipase is codon optimized for expression in a heterologous cell.

54. The expression vector of claim 53, wherein the heterologous cell is a Burkholderia cepacia, Burkholderia glumae, Pseudomonas fluorescens, Chromobacterium viscosum, Pseudomonas luteola, Pseudomonas fragi, or Escherichia coli cell.

55. A cell comprising the expression vector of any one of claims 52-54.

56. The cell of claim 55, wherein the cell is a Burkholderia cepacia, Burkholderia glumae, Pseudomonas fluorescens, Chromobacterium viscosum, Pseudomonas luteola, or Escherichia coli cell.

57. A method of producing a recombinant mutant microbial lipase enzyme, the method comprising growing the cell of claim 55 or 56 under conditions so that the host cell expresses the recombinant mutant microbial lipase enzyme, and purifying the recombinant mutant microbial lipase enzyme.

58. A pharmaceutical composition comprising the lipase of any one of claims 1-50, and a pharmaceutically acceptable carrier and/or an excipient.

59. The pharmaceutical composition of claim 58, further comprising a microbial protease, and/or a microbial amylase.

60. The pharmaceutical composition of claim 59, wherein the protease is an Aspergillus melleus protease and/or the amylase is an Aspergillus oryzae amylase.

61. The pharmaceutical composition of any one of claims 58-60, wherein the composition is formulated as an oral dosage form.

62. The pharmaceutical composition of any one of claims 58-61, wherein the composition is a formulated as a powder, granulate, pellet, micropellet, liquid, or a tablet.

63. The pharmaceutical composition of any one of claims 58-62, wherein the composition is encapsulated in a capsule or formulated as a tablet dosage form.

64. The pharmaceutical composition of any one of claims 58-63, wherein the composition does not comprise an enteric coating.

65. A method of treating a disease or disorder associated with a reduced ability to digest or absorb lipids, resulting in an elevated amount of undigested lipid, in a subject in need thereof, the method comprising administering to the subject an effective amount of the lipase of any one of claims 1-50, or the pharmaceutical composition of any one of claims 58-64, thereby treating the disease or disorder in the subject.

66. A method of treating maldigestion or malabsorption of lipids in a subject in need thereof, the method comprising administering to the subject an effective amount of the lipase of any one of claims 1-50, or the pharmaceutical composition of any one of claims 58-64, thereby treating the disease or disorder in the subject.

67. The method of claim 66, wherein the subject exhibits low level secretion of pancreatic enzymes or has a physiological condition that affects fat hydrolysis or fat absorption (e.g., reduced gastric, duodenal, liver, bile, or gallbladder function); reduced gastrointestinal transit, motility, mixing, emptying; or reduced intestinal mucosa function (e.g., induced by mucosal damage) that results in fat maldigestion or fat malabsorption or a fatty acid deficiency.

68. The method of claim 66 or 67, wherein the maldigestion or malabsorption of lipids is associated with a disease or disorder selected from exocrine pancreatic insufficiency (EPI), malabsorption syndrome, cystic fibrosis, chronic pancreatitis, acute pancreatitis, Schwachman-Diamond syndrome, a fatty acid disorder, Familial lipoprotein lipase deficiency, Johanson-Blizzard syndrome, Zollinger-Ellison syndrome, Pearson marrow syndrome, short-bowel syndrome, liver disease, primary biliary atresia, cholestasis, celiac disease, fatty liver disease, pancreatitis, diabetes, aging, cancer of the pancreas, stomach, small intestine, colon, rectal/anal, liver, hepatic, gallbladder, or, esophagus, cachexia, or a gastrointestinal disorder (e.g., Crohn's disease, irritable bowel syndrome, or ulcerative colitis), surgical invention of the stomach, small intestine, liver, gallbladder or pancreas.

69. A method of improving the absorption of fatty acids in a subject in need thereof, the method comprising administering to the subject an effective amount of the lipase of any one of claims 1-50, or the pharmaceutical composition of any one of claims 58-64, thereby improving absorption of fatty acids in the subject.

70. A method of increasing the amount of fatty acids in plasma, erythrocytes, or a tissue of a subject in need thereof, the method comprising administering to the subject an effective amount of the lipase of any one of claims 1-50, or the pharmaceutical composition of any one of claims 58-64, thereby increasing the amount of fatty acids in the subject.

71. A method of increasing the ratio of omega-3 to omega-6 fatty acids in plasma, erythrocytes, or a tissue of a subject in need thereof, the method comprising administering to the subject an effective amount of the lipase of any one of claims 1-50, or the pharmaceutical composition of any one of claims 58-64, thereby increasing the amount of fatty acids in the subject.

72. A method of reducing the amount of fatty acids in the stool of a subject in need thereof, the method comprising administering to the subject an effective amount of the lipase of any one of claims 1-50, or the pharmaceutical composition of any one of claims 50-56, thereby reducing the amount of fatty acids in the stool of the subject.

73. The method of any one of claims 65-72, wherein the fatty acids are long-chain poly-unsaturated fatty acids (LCPUFAs).

74. The method of any one of claims 65-73, wherein the fatty acids are omega-3 fatty acids.

75. The method of claim 74, wherein the omega-3 fatty acids are DHA, EPA, or DPA.

76. The method of any one of claims 65-75, wherein the subject is administered less than 400, 600, 800, or 1,000 mg of the lipase or pharmaceutical composition per day.

77. The method of any one of claims 65-76, wherein the lipase or pharmaceutical composition is administered in combination with a fat soluble vitamin (e.g., vitamin A, D, E, or K), an acid blocker, or a nutritional formula containing triglycerides.

78. The method of any one of claims 65-77, wherein the subject is a mammal.

79. The method of any one of claims 65-78, wherein the subject is a human.
